(19) United States
(12) Patent Application Publication
(10) Pub. No.: US 2022/0003768 A1
(43) Pub. Date: Jan. 6, 2022

(54) SEROLOGICAL BIOMARKERS FOR EARLY DIAGNOSIS OF LUNG CANCER

(71) Applicants: CDI LABORATORIES, INC., Mayaguez, PR (US); THE JOHNS HOPKINS UNIVERSITY, Baltimore, MD (US)

(72) Inventors: Ignacio PINO, Mayaguez, PR (US); Heng ZHU, Towson, MD (US); Jiang QIAN, Lutherville, MD (US); Yi HUANG, Fuzhou, Fujian (CN)

(21) Appl. No.: 17/282,723

(22) PCT Filed: Sep. 29, 2019
(86) PCT No.: PCT/CN2019/109104

§ 371 Date: Apr. 2, 2021

(30) Foreign Application Priority Data
Oct. 4, 2018 (CN) PCT/CN2018/109244

(51) Int. Cl. G01N 33/574; G01N 33/543

(52) U.S. Cl. CPC G01N 33/57423; G01N 33/54386; C12Y 301/03002; G01N 2333/4704; G01N 2333/916; G01N 2333/91194; C12Y 208/02002

(57) ABSTRACT

Biomarkers to screen for, identify, and/or characterize lung cancer in a subject with high selectivity and high specificity are disclosed. Also disclosed herein are methods for distinguishing lung cancer from another disease. Also disclosed herein are substrates, arrays, and reagents for use in the methods, and methods of their preparation.

### CROSS REFERENCE TO RELATED APPLICATIONS

This application claims priority to international application serial number PCT/CN2018/109244, filed on Oct. 4, 2018; the contents of which are hereby incorporated in their entirety.

### SUMMARY

Disclosed herein are methods comprising contacting a sample from a subject suspected of having cancer with one or more isolated and purified tumor-associated proteins. In some embodiments, the one or more isolated and purified tumor-associated proteins can comprise at least one selected from the group consisting of: an isolated and purified tumor-associated protein from Table 3, and a polypeptide having at least 80 percent sequence identity with a sequence as shown in one of SEQ ID NO: 121-149, and any combination thereof. In some embodiments, the one or more isolated and purified tumor-associated proteins thereof can comprise at least one selected from the group consisting of: tripartate motif containing 33 (TRIMM33), BCL tumor suppressor 7A (BCL7A), Prostatic acid phosphatase (ACPP), mitochondrial transcription termination factor 4 (MTERF4), Sulfotransferase family cytosolic 2B member 1 (SULT2B1), Tetratricopeptide repeat protein 1 (TTC1), a polypeptide having at least 80 percent sequence identity with a sequence as shown in one of SEQ ID NO: 121, SEQ ID NO: 124, SEQ ID NO: 126, SEQ ID NO: 141, SEQ ID NO: 142, SEQ ID NO: 149, and any combination thereof. In some embodiments the methods can additionally comprise detecting a binding of at least one of the one or more isolated and purified tumor-associated proteins to one or more moieties. In some embodiments, the cancer can be a lung cancer. In some embodiments, the methods can further comprise characterizing the subject as having an increased probability of having the lung cancer. In some embodiments, the characterizing can be performed with a sensitivity of at least about 60% or 70%. In some embodiments, the characterizing can be performed with a specificity of at least about 85%. In some embodiments, the one or more isolated and purified tumor-associated proteins can comprise less than about 100 sequentially unique proteins thereof. In some embodiments, the one or more isolated and purified tumor-associated proteins can comprise less than about 10 sequentially unique proteins. In some embodiments, the one or more isolated and purified tumor-associated proteins can comprise a tumor-associated protein that binds to immunoglobulin A (IgA) and/or a tumor-associated protein that binds to immunoglobulin G (IgG). In some embodiments, the moiety can be an antibody. In some embodiments, the antibody can be an autoantibody. In some embodiments, the detecting can comprise detecting a signal. In some embodiments, the signal can have a Z score greater than a cutoff value of 1. In some embodiments, the method can be a method of screening for the presence or absence of the cancer. In some embodiments, the detecting can be indicative of the subject having the cancer. In some embodiments, the methods can further comprise validating the presence of the cancer. In some embodiments, the methods can further comprise monitoring the cancer in the subject. In some embodiments, the method can distinguish the cancer from a second disease. In some embodiments, the detecting can be indicative of the subject having the cancer, wherein the patient displays at least one symptom that is characteristic of both the cancer and the second disease. In some embodiments, the second disease can be benign lung lesions (LBL), pneumonia, chronic obstructive pulmonary disease (COPD), pulmonary tuberculosis, or a second cancer. In some embodiments, the cancer can be rectal cancer, liver cancer, cervical cancer, esophagus cancer, or gastric cancer. In some embodiments, the lung cancer can be small-cell lung cancer (SCLC) or non-small cell lung cancer (NSLCLC). In some embodiments, the subject can have been previously diagnosed with the cancer. In some embodiments, the methods can further comprise selecting a therapeutic based on the result of the method. In some embodiments, the methods can further comprise administering a therapeutic based on the result of the method. In some embodiments, the methods can be comprised in a companion diagnostic test. In some embodiments, the methods can further comprise communicating the detecting via a communication medium. In some embodiments, the communication medium can comprise an electronic medium. In some embodiments, the subject can be a human. In some embodiments, the sample can comprise a body fluid. In some embodiments, the body fluid can comprise whole blood, plasma, serum, sputum, or plueral fluid. In some embodiments, the contacting can be performed on a solid support. In some embodiments, the contacting or the detecting can be performed using a portable device. In some embodiments, the one or more isolated and purified tumor-associated proteins can comprise: TRIM33, BCL7A, DDX4, CTAG1A, MAGEC2, and or MTERF4. In some embodiments, the one or more isolated and purified tumor-associated proteins can comprise: BCL7A, DDX4, CTAG1A, MAGEC2, MTERF4, and or NUDT14. In some embodiments, the one or more isolated and purified tumor-associated proteins can comprise: TRIM33, BCL7A, CTAG1A, MAGEC2, MTERF4, and or C1QTNF1. In some embodiments, the one or more isolated and purified tumor-associated proteins can comprise: BCL7A, DDX4, CTAG1A, MAGEC2, DDX4, and or RALGDS. In some embodiments, the one or more isolated and purified tumor-associated proteins can comprise: TRIM33, BCL7A, DDX4, CTAG1A, MTERF4, and C1QTNF1. In some embodiments, the one or more isolated and purified tumor-associated proteins can comprise: BCL7A, DDX4, CTAG1A, MAGEC2, MTERF4, and or APH1A. In some embodiments, the one or more isolated and purified tumor-associated proteins can comprise: BCL7A, DDX4, CTAG1A, MAGEC2, MTERF4, and or N4BP1. In some embodiments, the one or more isolated and purified tumor-associated proteins can comprise: BCL7A, DDX4, CTAG1A, MAGEC2, MTERF4, and or SSBP4. In some embodiments, the one or more isolated and purified tumor-associated proteins can comprise: TRIM33, BCL7A, DDX4, CTAG1A, APH1A, and or C1QTNF1. In some embodiments, the one or more isolated and purified tumor-associated proteins can comprise: TRIM33, BCL7A, DDX4, CTAG1A, KCNRG, and or C1QTNF1. In some embodiments, the one or more isolated and purified tumor-associated proteins can comprise: TRIM33, BCL7A, DDX4, CTAG1A, C1QTNF1, and or SSBP4. In some embodiments, the one or more isolated and purified tumor-associated proteins can comprise: BCL7A, DDX4, CTAG1A, MAGEC2, MTERF4, and or YP021. In some embodiments, the one or more isolated and purified tumor-associated proteins can comprise: BCL7A, DDX4, CTAG1A, MAGEC2, MTERF4, and or C1QTNF1. In some embodiments, the one or more isolated and purified tumor-associated proteins can comprise: BCL7A, DDX4, CTAG1A, MAGEC2, MTERF4, and or NUMBL. In some embodiments, the one or more isolated and purified tumor-associated proteins can comprise: BCL7A, DDX4, CTAG1A, MAGEC2, MTERF4, and or SULT2B1. In some embodiments, the one or more isolated and purified tumor-associated proteins can comprise: BCL7A, DDX4, CTAG1A, MTERF4, ACPP, and or C1QTNF1. In some embodiments, the one or more isolated and purified tumor-associated proteins can comprise: TRIM33, SSBP4, TP53, NSG1 and or CTAG1. In some embodiments, the one or more isolated and purified tumor-associated proteins can comprise: TRIM33, MTERF4, TP53, MAGEC2, and or CTAG1A.

Also disclosed herein are in vitro compositions that comprise at least two tumor-associated proteins, and a moiety bound to at least one of the at least two tumor-associated proteins. In some embodiments, the at least two tumor-associated proteins can comprise at least one selected from the group consisting of: tripartate motif containing 33 (TRIMM33), BCL tumor suppressor 7A (BCL7A), Prostatic acid phosphatase (ACPP), mitochondrial transcription termination factor 4 (MTERF4), Sulfotransferase family cytosolic 2B member 1 (SULT2B1), Tetratricopeptide repeat protein 1 (TTC1), a polypeptide having at least 80 percent sequence identity with a sequence as shown in one of SEQ ID NO: 121, SEQ ID NO: 124, SEQ ID NO: 126, SEQ ID NO: 141, SEQ ID NO: 142, SEQ ID NO: 149, and any combination thereof. In some embodiments, the moiety can be present in a sample obtained from a subject suspected of having cancer. In some embodiments, the at least two tumor-associated proteins can be isolated and purified. In some embodiments, the moiety can be an antibody. In some embodiments, the antibody can be an autoantibody. In some embodiments, the sample can comprise a body fluid. In some embodiments, the body fluid can comprise whole blood, plasma, or serum. In some embodiments, the subject is a human. In some embodiments, the cancer can be a lung cancer. In some embodiments, the at least two tumor-associated proteins can comprise: TRIM33, SSBP4, TP53, NSG1 and or CTAG1. In some embodiments, the at least two tumor-associated proteins can comprise: TRIM33, MTERF4, TP53, MAGEC2, and or CTAG1A.

Also disclosed herein are solid supports. In some embodiments, the solid supports can comprise two or more isolated and purified tumor-associated proteins attached thereto. In some embodiments, at least two of the two or more isolated and purified tumor-associated proteins can be selected from the group consisting of: tripartate motif containing 33 (TRIMM33), BCL tumor suppressor 7A (BCL7A), Prostatic acid phosphatase (ACPP), mitochondrial transcription termination factor 4 (MTERF4), Sulfotransferase family cytosolic 2B member 1 (SULT2B1), Tetratricopeptide repeat protein 1 (TTC1), a polypeptide having at least 80 percent sequence identity with a sequence as shown in one of SEQ ID NO: 121, SEQ ID NO: 124, SEQ ID NO: 126, SEQ ID NO: 141, SEQ ID NO: 142, SEQ ID NO: 149, and any combination thereof. In some embodiments, the two or more isolated and purified tumor-associated proteins can comprise less than about 100 sequentially unique proteins. In some embodiments, the two or more isolated and purified tumor-associated proteins can comprise less than about 10 sequentially unique proteins thereof. In some embodiments, the two or more isolated and purified tumor-associated proteins can comprise: TRIM33, SSBP4, TP53, NSG1 and or CTAG1. In some embodiments, the two or more isolated and purified tumor-associated proteins can comprise: TRIM33, MTERF4, TP53, MAGEC2, and or CTAG1A.

Also disclosed herein are devices. In some embodiments, the devices may comprise a memory that stores executable instructions, and a processor that executes the executable instructions such that the device can perform any of the methods described herein. In some embodiments, the devices may comprise any of the solid supports described herein, and a processor for detecting a signal. In some embodiments, the signal can be indicative of a binding of an autoantibody to at least one of the two or more isolated and purified tumor-associated proteins. In some embodiments, the signal can be at least about two standard deviations greater than a control signal.

Also disclosed herein are kits. In some embodiments, the kits may comprise any of the solid supports disclosed herein, and instructions for use of the kit. In some embodiments, the kits may comprise any of the devices disclosed herein, and instructions for use of the kit.

### INCORPORATION BY REFERENCE

All publications, patents, and patent applications herein are incorporated by reference in their entireties. In the event of a conflict between a term herein and a term in an incorporated reference, the term herein controls.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 depicts a schematic design of a study identifying serological biomarkers for lung cancer.

FIG. 2A depicts an example of a positive protein from a serum profiling assay, in which BCL7A was recognized by IgA antibodies of a LC patient. Fluorescence scanning and imaging results in a signal from the Cy3-linked anti-IgA antibody. FIG. 2B depicts an examples of a positive protein from a serum profiling assay, in which BCL7A was not recognized by IgA antibodies of a healthy patient. Fluorescence scanning and imaging results in an absence of signal from the Cy3-linked anti-IgA antibody. FIG. 2C depicts an example of a positive protein from a serum profiling assay, in which BCL7A was not recognized by IgA antibodies of a LBL patient. Fluorescence scanning and imaging results in an absence of signal from the Cy3-linked anti-IgA antibody. FIG. 2D depicts a box plot analysis of the HuProt array profiling of BCL7A in LC, healthy, and LBL patients.

FIG. 3A depicts an example of a positive protein from a serum profiling assay, in which MTERF4 was recognized by IgA antibodies of a LC patient. Fluorescence scanning and imaging results in a signal from the Cy3-linked anti-IgA antibody. FIG. 3B depicts an examples of a positive protein from a serum profiling assay, in which MTERF4 was not recognized by IgA antibodies of a healthy patient. Fluorescence scanning and imaging results in an absence of signal from the Cy3-linked anti-IgA antibody. FIG. 3C depicts an example of a positive protein from a serum profiling assay, in which MTERF4 was not recognized by IgA antibodies of a LBL patient. Fluorescence scanning and imaging results in an absence of signal from the Cy3-linked anti-IgA antibody. FIG. 3D depicts a box plot analysis of the HuProt array profiling of MTERF4 in LC, healthy, and LBL patients.

FIG. 4A depicts the signal distribution of BCL7A in early LC patients and a negative control group in a Phase II validation ELISA assay. The box plot shows that the signal intensity for BCL7A was significantly higher in the early LC group than in the control group. FIG. 4B depicts a Receiver Operating Characteristics (ROC) curve for BCL7A in the Phase II validation ELISA assay. The calculated Area Under the Curve (AUC) value of 0.731, sensitivity value of 0.489, and specificity value of 0.926 for BCL7A are shown as obtained at the optimal cutoff value for BCL7A.

FIG. 5A depicts the signal distribution of MTERF4 in early LC patients and a negative control group in a Phase II validation ELISA assay. The box plot shows that the signal intensity for FTERF4 was significantly higher in the early LC group than in the control group. FIG. 5B depicts a Receiver Operating Characteristics (ROC) curve for MTERF4 in the Phase II validation ELISA assay. The calculated Area Under the Curve (AUC) value of 0.684, sensitivity value of 0.222, and specificity value of 0.963 for MTERF4 are shown as obtained at the optimal cutoff value for MTERF4.

FIG. 6A depicts performance of a six biomarker panel in early LC patients in a Phase II ELISA Discovery assay. Dark gray and light gray lines represent samples scored as positives and negatives, respectively. A sample is predicted as LC positive when any one of the six proteins in this panel scored positive (i.e. k=1). FIG. 6B depicts performance of the biomarker panel in healthy patients in the Phase II ELISA Discovery assay. Dark gray and light gray lines represent samples scored as positives and negatives, respectively. A sample was predicted as LC positive when any one of the six proteins in this panel scored positive (i.e. k=1). FIG. 6C depicts performance of the biomarker panel in LBL patients in the Phase II ELISA Discovery assay. Dark gray and light gray lines represent samples scored as positives and negatives, respectively. A sample is predicted as LC positive when any one of the six proteins in this panel scored positive (i.e. k=1).

FIG. 7A. depicts performance of a biomarker panel early LC, Healthy, and LBL patients in a Phase II ELISA Validation assay. Dark gray and light gray lines represent samples scored as positives and negatives, respectively. A sample is predicted as LC positive when any one of the six proteins in this panel scored positive (i.e. k=1). FIG. 7B depicts performance of the biomarker panel in healthy patients in the Phase II ELISA Validation assay. Dark gray and light gray lines represent samples scored as positives and negatives, respectively. A sample was predicted as LC positive when any one of the six proteins in this panel scored positive (i.e. k=1). FIG. 7C depicts performance of the biomarker panel in LBL patients in the Phase II ELISA Validation assay Dark gray and light gray lines represent samples scored as positives and negatives, respectively. A sample was predicted as LC positive when any one of the six proteins in this panel scored positive (i.e. k=1)

FIG. 8 depicts an exemplary method for assessing LC in a subject.

### DETAILED DESCRIPTION

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

Some inventive embodiments herein contemplate numerical ranges. When ranges are present, the ranges include the range endpoints. Additionally, every sub range and value within the rage is present as if explicitly written out. The term “about” or “approximately” can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. The term “about” has the meaning as commonly understood by one of ordinary skill in the art. In some embodiments, the term “about” refers to ±10%. In some embodiments, the term “about” refers to ±5%.

Overview

Lung cancer (LC) remains the leading cause of mortality from malignant tumors. According to the World Health Organization (WHO), among the 8.8 million cancer-related deaths in 2015, LC caused 1.69 million deaths worldwide. In the most populated country, China, LC alone is responsible for the mortality of 42.05 per 100,000 persons. Recent studies have provided mounting evidence that cancer patients can exert humoral immune responses to autologous cellular antigens, dubbed as tumor associated antigens (TAAs), in a wide range of cancer types, including gastric cancer, lung cancer, and ovarian cancer. For example, autoantibodies against TAAs could be readily detected at the time of initial diagnosis of LC, and last up to several months or even years prior to clinical symptoms. Indeed, the IgG autoantibodies that target autoantigens, such as p53, CTAG1A, cyclin Y, ubiquilin 1, livin, and survivin, are readily detectable in serum samples collected from LC patients. Therefore, the discovery of non-invasive serological biomarkers for early stage LC diagnosis that yield high sensitivity and specificity holds great promise in intervention and prevention of LC.

Currently, screening and diagnosis of early stage LC mainly depends on high-resolution (or low-dose) chest computed tomography (CT) and biopsy. In some cases, chest CT and biopsy are not desirable tools due to the high-cost and invasiveness of the procedures. Alternatively, the detection of tumor antigens, such as CA125, CA199, neuron specific enolase (NSE), squamous cell carcinoma (SCC), carcinoembryonic antigen (CEA) and cytokeratin 19 fragment (CYFRA 21-1), in serum samples in LC patients, have been reported and suggested as diagnostic markers. However, these reports do not include specificity testing, and the sensitivity of CA125, CA199, NSE, SCC, CEA and CYFRA 21-1 is only 5.0%, 4.9%, 19.7%, 10.9%, 17.2%, and 26.5%, respectively, in patients with stage 1 NSCLC. In addition, tumor antigens such as CYFRA 21-1 are found elevated in serum samples of patients with radiation pneumonitis, and thus has limited their use in distinguishing LC from pneumonitis. Furthermore, many tumor antigens have been found in serum samples collected from patients having non LC cancers, raising concerns of non-specificity of these markers in LC diagnosis.

Detecting autoantibodies is a promising approach for early diagnosis of LC because humoral immune responses to autologous cellular antigens, called tumor associated antigens (TAAs), could exist several months or years prior to clinical symptoms. Tumor-associated autoantibodies often target the mutated, modified, or aberrantly expressed proteins in tumor cells, and could also act as immunologic indicators, uncovering molecular events of tumorigenesis. Several autoantibodies against TAAs, such as p53, ubiquilin 1, cyclin Y, livin, and survivin, have been found to be readily detectable in serum samples collected from LC patients. However, these reported autoantibody-based serological biomarkers do not provide sufficient sensitivity or specificity for LC diagnosis, let alone early LC diagnosis. Therefore, it is urgent and essential to identify better serological biomarkers as an economic, straightforward, and noninvasive method for the diagnosis of early stage LC with high sensitivity and specificity.

Described herein is a survey of HuProt arrays, comprising 20,240 human proteins, for autoantigens that bind to IgA and IgG. From this survey, it has been discovered that combining IgA autoantigens with autoantigens of other immunoglobulin classes, such as IgG, is surprisingly effective at identifying early stage LC in human patients. Also described herein are biomarker panels identified by HuProt arrays capable of early stage LC diagnosis with >70% sensitivity and >85% specificity. One example of an integrated biomarker panel comprises three IgA autoantigens (BCL7A, TRIM33, and MTERF4) and three IgG autoantigens (CTAG1A, DDX4, and MAGEC2), had a 72.5% sensitivity at 85% specificity for diagnosis of early stage LC in human patients. In some embodiments, a chemiluminescence platform can comprise TRIM33-IgA, SSBP4-IgA, TP53-IgG, NSG1-IgG, CTAG1A-IgG. In some embodiments, an ELISA platform can comprise TRIM33-IgA, MTERF4-IgA, TP53-IgG, MAGEC2-IgG, CTAG1A-IgG.

### Definitions

The terms “attach”, “bind”, “couple”, and “link” are used interchangeably and can refer to covalent interactions (e.g., by chemically coupling), or non-covalent interactions (e.g., ionic interactions, hydrophobic interactions, hydrogen bonds, hybridization, etc.). The terms “specific”, “specifically”, or specificity” can refer to the preferential recognition, contact, and formation of a stable complex between a first molecule and a second molecule compared to that of the first molecule with any one of a plurality of other molecules (e.g., substantially less to no recognition, contact, or formation of a stable complex between the first molecule and any one of the plurality of other molecules). For example, two molecules may be specifically attached, specifically bound, specifically coupled, or specifically linked. For example, specific hybridization between a first polynucleotide and a second polynucleotide can refer to the binding, duplexing, or hybridizing of the first polynucleotide preferentially to a particular nucleotide sequence of the second polynucleotide under stringent conditions. Sufficient number complementary base pairs in a polynucleotide sequence may be required to specifically hybridize with a target nucleic acid sequence. A high degree of complementarity may be needed for specificity and sensitivity involving hybridization, although it need not be 100%.

The term “about” or “approximately” can mean within an acceptable error range for the particular value as determined by one of ordinary skill in the art, which will depend in part on how the value is measured or determined, i.e., the limitations of the measurement system. For example, “about” can mean about plus or minus 10%, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “subject”, “patient” or “individual” as used herein can encompass a mammal and a non-mammal. A mammal can be any member of the Mammalian class, including but not limited to a human; a non-human primates such as a chimpanzee, an ape or other monkey species; a farm animal such as cattle, a horse, a sheep, a goat, a swine; a domestic animal such as a rabbit, a dog (or a canine), and a cat (or a feline); or a laboratory animal including a rodent, such as a rat, a mouse and a guinea pig, and the like. A non-mammal can include a bird, a fish and the like. In some embodiments, a subject can be a mammal. In some embodiments, a subject can be a human. In some instances, the human can be male or female. In some instances, the human can be an adult. In some instances, the human can be a child. In some instances, the human can be age 0-17 years old. In some instances, the human can be age 18-130 years old. In some instances, the subject is diagnosed with, or is suspected of having, a condition or disease such as cancer. In instances, the subject is diagnosed with, or is suspected of having lung cancer.

The term “non-diseased subject” as used herein can encompass a healthy individual. A healthy individual can be an individual without a particular disease. For example, a healthy individual can be an individual without a cancer such as lung cancer.

The terms “treat,” “treating”, “treatment,” “ameliorate” or “ameliorating” and other grammatical equivalents as used herein, can include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms can further include achieving a therapeutic benefit and/or a prophylactic benefit. Therapeutic benefit can mean eradication or amelioration of the underlying disease being treated. Also, a therapeutic benefit can be achieved with the eradication or amelioration of one or more of the physiological symptoms associated with the underlying disease such that an improvement can be observed in the patient, notwithstanding that, in some embodiments, the patient can still be afflicted with the underlying disease.

The term “isolated” as used herein refers to the removal of a protein, such as a tumor-associated protein, from its endogenous or natural environment. For example, a protein may be isolated by removal and separation from an organism, bodily fluid, or fermentation medium.

The term “purified” as used herein can define the purity of a protein, such as a tumor-associated protein, and can refers to proteins that are substantially free of other human or non-human proteins of natural or endogenous origin. A purified protein may contain less than about 20%, 15%, 10%, 5%, 4%, 3%, 2%, or 1% by mass of protein contaminants residual of the purification process. Purified proteins, however, may contain other proteins added as stabilizers, carriers, excipients, binding agents, or purification tags. In some aspects, purified proteins may be detectable as a substantially single band on a polyacrylamide gel using a silver or Coomasie stain.

The term “probe” as used herein can refer to any molecular moiety that can be identified to detect the presence of the probe. A probe may be used to detect a target molecule or moiety to which the probe is associated. The association of a probe to a target may be covalent or non-covalent. In some embodiments, a probe may comprise any detectable moiety, such as a fluorescent dye, a phosphor, a radiolabel, or a chromophore. The term “fluorescent dye” as used herein can refer to molecular moiety comprising a fluorophore that can be used as a probe. In some aspects, a chromophore may absorb, reflect, or emit light at ultraviolet or visible wavelengths.

The terms “early stage” and “late stage” as used herein can refer to the degree of progression of a cancer. For example, an early stage lung cancer can be a stage I lung cancer or a stage II lung cancer. In some instances, an early stage lung cancer can be confined to the lung tissue of a subject. In some instances, an early stage lung cancer can be a limited stage small cell lung cancer. In some instances, a late stage lung cancer can be a stage III or stage IV lung cancer. In some instances, a late stage lung cancer can have spread from the lung tissue to another organ of a subject. In some instances, a late stage lung cancer can be an extensive stage small cell lung cancer.

The terms “homologous,” “homology,” or “percent homology” as used herein can refer to the degree of sequence similarity between an amino acid or nucleotide sequence and a reference sequence. As used herein, the term “homology” can be used interchangeably with the term “identity.” In some instances, percent sequence homology can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application. In some instances, percent homology of sequences can be determined using Smith-Waterman homology search algorithm. Smith & Waterman (1981) Adv. Appl. Math. 2: 482-489.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues. Most sequence comparison method over longer sequences are designed to produce optimal alignments that take into consideration possible insertions and deletions without penalizing unduly the overall homology score. This is achieved by inserting “gaps” in the sequence alignment to try to maximize local homology. these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimized alignments with fewer gaps. Most alignment programs allow the gap penalties to be modified. In some embodiments, typically the default values are used when using such software for sequence comparisons. In some embodiments, calculation of maximum % homology can therefore firstly requires the production of an optimal alignment, taking into consideration gap penalties. Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. In some instances, an alignment is determined by the Smith-Waterman homology search algorithm using an affine gap search with a gap open penalty of 12 a gap extension penalty of 2, and a blocks substitution matrix (BLOSUM) of 62.

Samples

In some embodiments, the sample can be an antibody containing sample. In some embodiments, an antibody containing sample can be a biological fluid. In some embodiments, the sample can be a biological fluid. A biological fluid prepared for analysis in the process described herein include or can include a host of potential biomarkers including markers expressed on cells (non-adherent cells, including T-cells or other immune effector cells), microorganisms, proteins, peptides, lipids, polysaccharides, small molecules, organic molecules, inorganic molecules, biological molecules and including any detectable or reactable moiety in such complex milieu. In some embodiments, such antibodies and, in particular, can be antibodies generated as a result of a disease or condition. In some embodiments, body fluids such as serum, plasma, saliva or other fluids or samples derived from a subject or animal or organism can be the source of such biomarkers. In some embodiments, the sample can be blood, serum, saliva or CSF. In some embodiments, the sample can be for example, sputum, bone marrow, synovial fluid, aqueous humor, amniotic fluid, cerumen, breast milk, broncheoalveolar lavage fluid, semen, prostatic fluid, Cowper's fluid, pre-ejaculatory fluid, female ejaculate, sweat, tears, cyst fluid, pleural fluid, peritoneal fluid, pericardial fluid, lymph, chyme, chyle, bile, interstitial fluid, menses, pus, sebum, vaginal secretion, mucosal secretion, stool water, pancreatic juice, lavage fluid from sinus cavities, bronchopulmonary aspirate, blastocyl cavity fluid, or umbilical cord blood. In some embodiments, a sample can be a cell-free nucleic acid sample. In some embodiments, a sample can comprise circulating cell free nucleic acid. In some embodiments, a sample can be from a subject afflicted with a disease or condition disclosed herein. In some embodiments, a subject from which a sample is obtained can have a disease. In some embodiments, the disease is a cancer. In some embodiments, the cancer can be lung cancer. In some embodiments, the lung cancer can be SCLC. In some embodiments, the SCLC can be limited stage SCLC or extensive stage SCLC. In some embodiments, the lung cancer can be NSCLC. In some embodiments, the NSCLC can be occult stage, stage 0, stage I, stage II, stage III, stage IV.

Biomarkers

A biomarker can refer to a biomolecule. In some cases, a biomarker can be a biomolecule associated with a disease. When associated with a disease, a biomarker can have a profile different under the disease condition compared to a non-disease condition. A biomarker can be a modification. A biomarker can be an epigenetic modification. A biomarker can be any class of biomolecules, including polynucleotides, polypeptides, carbohydrates and lipids. In some cases, a biomarker can be a polynucleotide. In some cases, a biomarker can be a polypeptide. In some cases, a biomarker can be an antibody. A polynucleotide can be any type of nucleic acid molecule, including DNA, RNA, a hybridization thereof, or any combination thereof. For example, a polynucleotide can be cDNA, genomic DNA, mRNA, tRNA, rRNA, or microRNA. In some cases, a polynucleotide can be a cell-free nucleic acid molecule circulating in blood or a cellular nucleic acid molecule in a cell circulating in blood. A polypeptide or protein can be contemplated to include any fragments thereof, in particular, immunologically detectable fragments. A biomarker can also include one or more fragments of the biomarker having sufficient sequence such that it still possesses the same or substantially the same function as the full-size biomarker. An active fragment of a biomarker retains 100% of the activity of the full-size biomarker, or at least about 99%, 95%, 90%, 85%, 80% 75%, 70%, 65%, 60%, 55%, or at least 50% of its activity. In certain cases, an active fragment of a biomarker can be detectable (e.g., a polypeptide detectable by an antibody, or a polynucleotide detectable by an oligonucleotide). A biomarker of a disease disclosed herein (e.g. cancer) can be a biomolecule associated with a disease. In some cases, a biomarker of a disease can be a biomolecule associated with the disease, but not associated with other diseases. In some cases, a biomarker of a disease can be a biomolecule associated with that disease and other diseases. In some embodiments, a biomarker can be a tumor-associated protein or fragment thereof. In some embodiments, a biomarker can be tumor protein p53 (p53), ethylmalonic encephalopathy protein 1 (ETHE1), Cancer/Testis Antigen 1A (CTAG1A), C1q And Tumor Necrosis Factor Related Protein 1 (C1QTNF1), Testis Expressed 264 (TEX264), Claudin 2 (CLDN2), Neuron Specific Gene Family Member 1 (NSG1), GTPase HRas (HRas), Cytoskeleton Associated Protein 2 (CKAP2), Dipeptidyl peptidase 4 (DPP4), Calcium-binding protein 39 (CAB39), Centromere protein X (STRA13), LIM Zinc Finger Domain Containing 1 (LIMS1), Recombination Signal Binding Protein for Immunoglobulin Kappa J Region 1 (RBPJ1), Recombination Signal Binding Protein for Immunoglobulin Kappa J Region 2 (RBPJ2), Recombination Signal Binding Protein for Immunoglobulin Kappa J Region 3 (RBPJ3), Protein MAK16 Homolog (MAK16), Calcium-Binding and Coiled-Coil Domain-Containing Protein 2 (CALCOCO2), Syndecan Binding Protein (SDCBP), Galectin-8 (LGALS8), Doublesex and Mab-3 Related Transcription Factor 2 (DMRT2), or a fragment of any of the above.

Tumor-Associated Proteins

A tumor associated protein can be an antigenic substance produced in or by tumor cells. A tumor-associated protein may be known as a tumor-associated antigen. A tumor associated protein can trigger an immune response in a subject. A tumor-associated antigen can be a protein that is not exposed to the immune system in a healthy subject. Tumor-associated proteins may be sequestered from the immune system in a healthy subject. Tumor-associated proteins may be expressed in small quantities in a healthy subject. Tumor associated proteins may be expressed in greater quantities in a cancerous cell than in a non-cancerous cell. Tumor associated proteins may be expressed only in certain stages of development. A tumor-associated protein may be a oncofetal antigen. Tumor-associated proteins may be structurally modified from the sequence found in a healthy subject due to mutation. A tumor-associated protein may be the mutated form of a naturally expressed protein. A tumor-associated protein may be the un-mutated form of a naturally expressed protein. In some instances, a tumor associated protein may comprise at least 1, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, 200, 250, 300, 350, 400, 450, 500, 600, 700, 800, 900, 1000, 2000, 3000, 4000, 5000, 6000, 7000, 8000, 9000, 10000, 15000, 20000, 25000, 30000, 35000, or 40000 amino acids.

A tumor-associated protein may be present exclusively in tumor cells. A tumor-associated protein may be present in tumor cells and in healthy cells. In some instances, a tumor-associated protein may be expressed from a protooncogene. In some instances, a tumor-associated protein may be expressed from a tumor-suppressor gene. A tumor-associated protein may be expressed in a cell infected with an oncovirus. In some instances, a tumor-associated protein may be recognized by a subject's immune system. A tumor-associated protein may be recognized by an antibody, such as an autoantibody. In some embodiments, a tumor-associated protein may be over express in a subject with a disease. In some embodiments, a tumor-associated protein expression may be reduced in a subject with a disease.

A tumor-associated protein can be, but is not limited to, a polypeptide, a protein, a protein fragment, a tagged protein, a fusion protein, an antibody, or an antibody fragment. In some instances, a tumor-associated protein can comprise at least two amide bonds. In some instances, a tumor-associated protein does not comprise a phosphodiester linkage. In some instances, a tumor-associated protein is not DNA or RNA. In some aspects one or more tumor-associated proteins can be spotted onto an array. In some instances, a tumor-associated protein may be glycosylated or methylated or hydroxymethylated.

In some instances, a tumor-associated protein can comprise a polypeptide, protein, or fragment thereof “Polypeptide” and “protein” are used interchangeably and can refer to a polymer of two or more amino acids joined by a covalent bond (e.g., an amide bond). Polypeptides as described herein can include full length proteins (e.g., fully processed proteins) as well as shorter amino acid sequences (e.g., fragments of naturally-occurring proteins or synthetic polypeptide fragments). Polypeptides can include naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V) and non-naturally occurring amino acids (e.g., amino acids which is not one of the twenty amino acids commonly found in peptides synthesized in nature, including synthetic amino acids, amino acid analogs, and amino acid mimetics). A tumor-associated protein can be a biomarker.

For example, a tumor-associated protein can comprise an isolated polypeptide, a purified polypeptide, or a polypeptide within a virus particle. For example, a tumor-associated protein can comprise a polypeptide within a virus particle membrane. A virus particle can refer to a fully or partially assembled capsid of a virus surrounded by a lipid envelope. A viral particle may or may not contain nucleic acids.

For example, a tumor-associated protein can comprise an antibody or fragment thereof. For example, a tumor-associated protein can comprise a transcription factor. For example, a tumor-associated protein can comprise a receptor. For example, a tumor-associated protein can comprise a transmembrane receptor.

Tumor-associated proteins can include isolated, purified, and/or recombinant polypeptides. Tumor-associated proteins can include tumor-associated proteins present in a mixture of analytes (e.g., a lysate). For example, tumor-associated proteins can include tumor-associated proteins present in a lysate from a plurality of cells or from a lysate of a single cell.

A tumor-associated protein can comprise a member of a specific binding pair (e.g., a ligand). A tumor-associated protein can be monovalent (monoepitopic) or polyvalent (polyepitopic). A tumor-associated protein can be antigenic or haptenic. A tumor-associated protein can be a single molecule or a plurality of molecules that share at least one common epitope or determinant site. A tumor-associated protein can be a part of a cell (e.g., a bacteria cell, a plant cell, or an animal cell). A target cell can be either in a natural environment (e.g., tissue), a cultured cell, or a microorganism (e.g., a bacterium, fungus, protozoan, or virus), or a lysed cell. A tumor-associated protein can be further modified (e.g. chemically), to provide one or more additional binding sites such as, but not limited to, a dye (e.g., a fluorescent dye), a polypeptide modifying moiety such as a phosphate group, a carbohydrate group, and the like, or a polynucleotide modifying moiety such as a methyl group.

A tumor-associated protein can comprise at least one potential binding site for an antibody. In some instances, a tumor-associated protein comprises one binding site. In some instances, a tumor-associated protein comprises at least two binding sites. For example, a tumor-associated protein can comprise 2, 3, 4, 5, 6, 7, 8, 9, 10, 11, 12, 13, 14, 15, 16, 17, 18, 19, 20, or more binding sites.

In some instances, a tumor-associated protein can be a molecule found in a sample from a host. A sample from a host includes a body fluid (e.g., urine, blood, plasma, serum, saliva, semen, stool, sputum, cerebral spinal fluid, tears, mucus, and the like). A sample can be examined directly or may be pretreated to render the tumor-associated protein more readily detectible. Samples include a quantity of a substance from a living thing or formerly living things. A sample can be natural, recombinant, synthetic, or not naturally occurring. A tumor-associated protein can be expressed in a cell naturally or recombinantly, in a cell lysate or cell culture medium, an in vitro translated sample, or an immunoprecipitation from a sample (e.g., a cell lysate).

In some instances, a tumor-associated protein is expressed in a cell-free system or in vitro. For example, a tumor-associated protein can be in a cell extract containing a nucleotide template and raw materials for translation of the tumor-associated protein. In some instances, a tumor-associated protein can be in a cell extract containing a DNA template, and reagents for transcription and translation. Exemplary sources of cell extracts that can be used include wheat germ, Escherichia coli, rabbit reticulocyte, hyperthermophiles, hybridomas, Xenopus oocytes, insect cells, and mammalian cells (e.g., human cells). Exemplary cell-free methods that can be used to express tumor-associated proteins (e.g., to produce tumor-associated protein on an array) include Protein in situ arrays (PISA), Multiple spotting technique (MIST), Self-assembled mRNA translation, Nucleic acid programmable protein array (NAPPA), nanowell NAPPA, DNA array to protein array (DAPA), membrane-free DAPA, nanowell copying and μIP-microintaglio printing, and pMAC—protein microarray copying (See Kilb et al., Eng. Life Sci. 2014, 14, 352-364).

In some instances, a tumor-associated protein is synthesized in situ (e.g., on a solid substrate of an array) from a DNA template. In some instances, a plurality of tumor-associated proteins is synthesized in situ from a plurality of corresponding DNA templates in parallel or in a single reaction. Exemplary methods for in situ tumor-associated protein expression include those described in Stevens, Structure 8(9): R177-R185 (2000); Katzen et al., Trends Biotechnol. 23(3):150-6. (2005); He et al., Curr. Opin. Biotechnol. 19(1):4-9. (2008); Ramachandran et al., Science 305(5680):86-90. (2004); He et al., Nucleic Acids Res. 29(15):E73-3 (2001); Angenendt et al., Mol. Cell Proteomics 5(9): 1658-66 (2006); Tao et al, Nat Biotechnol 24(10):1253-4 (2006); Angenendt et al., Anal. Chem. 76(7):1844-9 (2004); Kinpara et al., J. Biochem. 136(2):149-54 (2004); Takulapalli et al., J. Proteome Res. 11(8):4382-91 (2012); He et al., Nat. Methods 5(2):175-7 (2008); Chatterjee and J. LaBaer, Curr Opin Biotech 17(4):334-336 (2006); He and Wang, Biomol Eng 24(4):375-80 (2007); and He and Taussig, J. Immunol. Methods 274(1-2):265-70 (2003).

In some instances, tumor-associated protein synthesis can be carried out on a solid surface (e.g., an array surface) coated with a protein-capturing reagent or antibody. In some instances, a tumor-associated protein can comprise a tag (e.g., polyhistidine or GST) that is bound by the capture reagent or antibody, thus coupling the tumor-associated protein to the solid surface (e.g., a nucleic acid programmable protein array (NAPPA)). In some instances, a DNA template is immobilized onto the same protein-capture surface. For example, the DNA template can be biotinylated and bound to avidin pre-coated onto the protein capture surface. In some instances, the DNA template is not coupled to the solid support. In some instances, the DNA template is added as a free molecule in the reaction synthesis mixture (e.g., a protein in situ array (PISA)).

In some instances, in situ puromycin-capture methods can be used to express tumor-associated protein. For example, the template DNA can be transcribed to mRNA, and a single-stranded DNA oligonucleotide modified with biotin and puromycin on each end can be hybridized to the 3′-end of the mRNA. The mRNAs can be coupled to the surface e.g., by the binding of biotin to streptavidin that is pre-coated on the surface. Cell extract can then be added to initiate in situ translation. When the ribosome reaches the hybridized oligonucleotide, it stalls and incorporates the puromycin molecule to the nascent polypeptide chain, thereby attaching the newly synthesized protein to the surface via the DNA oligonucleotide. Purified tumor-associated proteins may be obtained after the mRNA is removed (e.g., digested with RNase).

In some instances, DNA array to protein array (DAPA) methods can be used to repeatedly produce protein arrays by printing them from a single DNA template array, on demand. An array of immobilized DNA templates on a substrate is assembled face-to-face with a second substrate pre-coated with a protein-capturing reagent, and a membrane soaked with a cell extract is placed between the two substrates for transcription and translation. The synthesized tumor-associated proteins are then immobilized onto a substrate to form the array.

An array can comprise a plurality of tumor-associated proteins. An array can comprise a plurality of tumor-associated proteins representing a substantial portion or an entire organism's proteome, such as a bacterial, viral, fungal, plant, or animal proteome. An array can comprise a plurality of tumor-associated proteins representing a substantial portion or an entire proteome of an insect or mammal, such as a mouse, rat, rabbit, cat, dog, monkey, goat, or human. For example, an array can comprise a plurality of tumor-associated proteins representing at least 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or 100% of an organism's proteome.

An array can comprise at least one tumor-associated protein. A array can comprise a plurality of tumor-associated proteins comprising at least 2 different tumor-associated proteins. For example, a tumor-associated protein can comprise a plurality of tumor-associated protein comprising at least 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, or 25,000 different tumor-associated proteins. For example, an array can comprise a plurality of tumor-associated proteins comprising less than 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, or 25,000 different tumor-associated proteins.

In some instances, tumor-associated proteins can comprise a tag. In some embodiments, the tag is an affinity tag. Examples of such affinity tags include, but are not limited to, Glutathione-S-transferase (GST), Maltose binding protein (MBP), Green Fluorescent Protein (GFP), AviTag (a peptide allowing biotinylation by the enzyme BirA and so the protein can be isolated by streptavidin), Calmodulin-tag (a peptide bound by the protein calmodulin), polyglutamate tag (a peptide binding efficiently to anion-exchange resin such as Mono-Q), FLAG-tag (a peptide recognized by an antibody), HA-tag (a peptide recognized by an antibody), His tag (generally 5-10 histidines which are bound by a nickel or cobalt chelate), Myc-tag (a short peptide recognized by an antibody, S-tag, SBP-tag (a peptide which binds to streptavidin), Softag 1, Strep-tag (a peptide which binds to streptavidin or the modified streptavidin called streptactin), TC tag (a tetracysteine tag that is recognized by FlAsH and ReAsH biarsenical compounds), V5 tag, Xpress tag, Isopeptag (a peptide which binds covalently to pilin-C protein), SpyTag (a peptide which binds covalently to SpyCatcher protein) or a combination thereof. In some instances, for example, tumor-associated protein can comprise a fusion tag. For example, a tumor-associated protein can comprise a GST-tag, His-tag, FLAG-tag, T7 tag, S tag, PKA tag, HA tag, c-Myc tag, Trx tag, Hsv tag, CBD tag, Dsb tag, pelB/ompT, KSI, MBP tag, VSV-G tag, 3-Gal tag, GFP tag, or a combination thereof, or other similar tags. In some instances, the protein tag binder is a group which binds an endogenous protein tag (e.g., an epitope on the protein). In this group of embodiments, the protein tag binder can typically be an antibody or antibody fragment which is sufficient to form a non-covalent association complex with the protein tag or epitope. In some embodiments, the tumor-associated proteins comprise PTMs including, but not limited to, glycosylation, phosphorylation, acetylation, methylation, myristoylation, prenylation, or proteolytic processing. In some embodiments, a tumor-associated protein is homologous to a native polypeptide.

In some instances, a tumor-associated protein can comprises a contiguous span of at least 6 amino acids, for example, least 8, 9, 10, 12, 15, 20, 25, 30, 40, 50, or 100 amino acids of a reference sequence. In some instances, a tumor-associated protein comprises a contiguous stretch of amino acids comprising a site of a mutation or functional mutation, including a deletion, addition, swap, or truncation of the amino acids in a polypeptide sequence. In some embodiments a tumor-associated protein is truncated. Polypeptides may be isolated from human or mammalian tissue samples or expressed from human or mammalian genes. Polypeptides may be made using routine expression methods known in the art. A polynucleotide encoding a desired polypeptide may be inserted into an expression vector suitable for any convenient host. Both eukaryotic and prokaryotic host systems can be used in forming recombinant polypeptides. A polypeptide may be isolated from lysed cells or from the culture medium and purified to the extent needed for its intended use. (See, e.g., WO2012103260 and WO2011159959). Purification may be by any technique known in the art, for example, differential extraction, salt fractionation, chromatography, centrifugation, and the like (See, e.g., Abbondanzo et al., (1993) Methods in Enzymology, Academic Press, New York. pp. 803-23).

In addition, shorter protein fragments may be produced by chemical synthesis. Alternatively, proteins of the presently disclosed subject matter are extracted from cells or tissues of humans or non-human animals. Methods for purifying proteins are known in the art, and include the use of detergents or chaotropic agents to disrupt particles followed by differential extraction and separation of the polypeptides by ion exchange chromatography, affinity chromatography, sedimentation according to density, and gel electrophoresis, for example. Reference cDNA may be used to express polypeptides. A nucleic acid encoding a polypeptide to be expressed can be operably linked to a promoter in an expression vector using conventional cloning technology. For example, a polypeptide in an expression vector may comprise the full coding sequence for the polypeptide or a portion thereof.

In some embodiments, a tumor-associated protein can be a membrane bound protein. In one embodiment, the membrane bound protein is CD4, a classical type I membrane protein with a single transmembrane (TM) domain. (Carr et al., (1989) J. Biol. Chem. 264:21286-95). In another embodiment, the membrane bound protein is GPR77, a multi-spanning, G-protein coupled receptor (GPCR) membrane protein. (Cain & Monk, (2002) J. Biol. Chem. 277:7165-69).

Additional exemplary membrane bound proteins include, but are not limited to, GPCRs (e.g. adrenergic receptors, angiotensin receptors, cholecystokinin receptors, muscannic acetylcholine receptors, neurotensin receptors, galanin receptors, dopamine receptors, opioid receptors, erotonin receptors, somatostatin receptors, etc.), ion channels (e.g., nicotinic acetylcholine receptors, sodium channels, potassium channels, etc.), receptor tyrosine kinases, receptor serine/threonine kinases, receptor guanylate cyclases, growth factor and hormone receptors (e.g., epidermal growth factor (EGF) receptor), and others. Mutant or modified variants of membrane-bound proteins may also be used. For example, some single or multiple point mutations of GPCRs retain function and are involved in disease (See, e.g., Stadel et al., (1997) Trends in Pharmacological Review 18:430-37). Also provided herein are libraries of tumor-associated proteins comprising a plurality of tumor-associated proteins in which at least 5%, 10%, 20%, 30%, 40%, 50%, 60%, 70%, 80%, 90%, 99% or 100% of the tumor-associated proteins are part of a common pathway. The present invention also provides a library of tumor-associated proteins in which the library represents at least 5%, 10%, 20%, 30%, 40%, 50%, 60%, 70%, 80%, 90%, 99% or 100% of all the tumor-associated proteins that are part of a common pathway in the genome.

In some embodiments, a tumor-associated protein can be tumor protein p53 (p53), ethylmalonic encephalopathy protein 1 (ETHE1), Cancer/Testis Antigen 1A (CTAG1A), C1q And Tumor Necrosis Factor Related Protein 1 (C1QTNF1), Testis Expressed 264 (TEX264), Claudin 2 (CLDN2), Neuron Specific Gene Family Member 1 (NSG1), GTPase HRas (HRas), Cytoskeleton Associated Protein 2 (CKAP2), Dipeptidyl peptidase 4 (DPP4), Calcium-binding protein 39 (CAB39), Centromere protein X (STRA13), LIM Zinc Finger Domain Containing 1 (LIMS1), Recombination Signal Binding Protein for Immunoglobulin Kappa J Region 1 (RBPJ1), Recombination Signal Binding Protein for Immunoglobulin Kappa J Region 2 (RBPJ2), Recombination Signal Binding Protein for Immunoglobulin Kappa J Region 3 (RBPJ3), Protein MAK16 Homolog (MAK16), Calcium-Binding and Coiled-Coil Domain-Containing Protein 2 (CALCOCO2), Syndecan Binding Protein (SDCBP), Galectin-8 (LGALS8), Doublesex and Mab-3 Related Transcription Factor 2 (DMRT2), or a fragment of any of the above.

In some embodiments, a tumor-associated protein can be Synovial sarcoma translocated to X chromosome protein (SS18), Putative uncharacterized protein MGC34800 (YP021), Gamma-secretase subunit APH-1A (APH1A), TOM1-like protein 2 (TOM1L2), Phosphatidylinositol 5-phosphate 4-kinase type-2 gamma (PIP4K2C), Numb-like protein (NUMBL), Uridine diphosphate glucose pyrophosphatase (NUDT14), Ral guanine nucleotide dissociation stimulator (RALGDS), Leukosialin (SPN), 60S acidic ribosomal protein P1 (RPLP1), Serine/threonine-protein kinase DCLK1 (DCLK1), Sulfotransferase family cytosolic 2B member 1 (SULT2B1), Chromosome 14 open reading frame 37 protein (C14orf37), Melanoma-associated antigen 1 (MAGEA8), Probable ATP-dependent RNA helicase DDX4 (DDX4), B-cell CLL/lymphoma 7 protein family member A (BCL7A), Nucleolar protein 3 (NOL3), Single-stranded DNA-binding protein 4 (SSBP4), Thioredoxin domain-containing protein 2 (TXNDC2), Putative fidgetin-like protein 2 (FIGNL2), Janus kinase and microtubule-interacting protein 2 (JAKMIP2), Potassium channel regulatory protein (KCNRG), NEDD4-binding protein 1 (N4BP1), Prostatic acid phosphatase (ACPP), Tripartite motif-containing 33 (TRIM33), Mitochondrial transcription termination factor MTERF4 (MTERF4), Tetratricopeptide repeat protein 1 (TTC1), Melanoma-associated antigen C2 (MAGEC2), or a fragment of any of the above.

In some embodiments, a tumor-associated protein can be a protein having 70%, 75%, 80%, 85%, 90%, 95%, 96%, 97%, 98%, 99%, or 99.5% sequence identity to SEQ ID NO: 1 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 2 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 3 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 4 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 5 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 6 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 7 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 8 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 9 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 10 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 11 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 12 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 13 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 14 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 15 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 16 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 17 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 18 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 19 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 20 or a fragment thereof. In some embodiments, a tumor-associated protein can be a protein having 70%, 75%, 80%, 85%, 90%, 95%, 96%, 97%, 98%, 99%, or 99.5% sequence identity with a sequence as shown in SEQ ID NO: 21 or a fragment thereof.

In some embodiments, a tumor-associated protein can be a protein having 70%, 75%, 80%, 85%, 90%, 95%, 96%, 97%, 98%, 99%, or 99.5% sequence identity to SEQ ID NO: 121 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 122 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 123 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 124 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 125 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 126 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 127 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 128 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 129 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 130 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 131 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 132 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 133 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 134 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 135 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 136 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 137 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 138 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 139 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 140 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 141 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 142 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 143 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 144 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 145 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 146 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 147 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 148 or a fragment thereof. In some embodiments, a tumor-associated protein can be a protein having 70%, 75%, 80%, 85%, 90%, 95%, 96%, 97%, 98%, 99%, or 99.5% sequence identity to SEQ ID NO: 149 or a fragment thereof.

In some embodiments, a tumor-associated protein can be a protein having 70%, 75%, 80%, 85%, 90%, 95%, 96%, 97%, 98%, 99%, or 99.5% sequence identity to a protein listed in Table 3, 5, or 6. In some embodiments, a tumor-associated protein can be a protein having 70%, 75%, 80%, 85%, 90%, 95%, 96%, 97%, 98%, 99%, or 99.5% sequence identity to any protein disclosed herein.

Tumor associated proteins may bind to one or more immunoglobulin classes of antibodies. For example, a tumor-associated protein may bind to an IgA or an IgG autoantibody. A tumor-associated protein may be referred to as an autoantigen of the immunoglobulin that it binds to. For example, a tumor-associated protein that can bind to an IgA may be referred to as an IgA autoantigen, and a tumor-associated protein that can bind to an IgG may be referred to as an IgG autoantigen, etc.

In some embodiments, tumor associated proteins may be combined into a panel comprising one or more tumor associated proteins, such that a sample from a subject suspected of having cancer may be contacted with the one more tumor-associated proteins or fragments thereof. In some embodiments, panel comprising one or more tumor-associated proteins or fragments thereof may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more tumor-associated proteins or fragments thereof.

In some embodiments, the one or more isolated and purified tumor-associated proteins or fragments thereof can comprise any of the combinations of tumor-associated proteins listed in Table 7 or fragments thereof. In some embodiments, the one or more isolated and purified tumor-associated proteins or fragments thereof can comprise: MTERF4 or a fragment thereof, MAGEC2 or a fragment thereof, TXDNC2 or a fragment thereof, BCL7A or a fragment thereof, FIGNL2 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and YP021 or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, KCNRG or a fragment thereof, and C1QTNF1 or a fragment thereof. In some embodiments, the one or more isolated and purified tumor-associated proteins or fragments thereof can comprise: ACPP or a fragment thereof, DCLK1 or a fragment thereof, SS18 or a fragment thereof, TOM1L2 or a fragment thereof, C14ORF37 or a fragment thereof, and NOL3 or a fragment thereof. In some embodiments, the one or more isolated and purified tumor-associated proteins or fragments thereof can comprise: TRIM33 or a fragment thereof, BCL7A or a fragment thereof, CTAG1A or a fragment thereof, MAGEC2 or a fragment thereof, MTERF4 or a fragment thereof, and RALGDS or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, RPLP1 or a fragment thereof, and N4BP1 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MAGEA8 or a fragment thereof, and HRAS or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, APH1A or a fragment thereof, and C1QTNF1 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and C1QTNF1 or a fragment thereof. In some embodiments, the one or more isolated and purified tumor-associated proteins or fragments thereof can comprise: MAGEC2 or a fragment thereof, TRIM33 or a fragment thereof, TXDNC2 or a fragment thereof, SULT2B1 or a fragment thereof, SPN or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MTERF4 or a fragment thereof, NUDT14 or a fragment thereof, and APH1A or a fragment thereof. In some embodiments, the one or more isolated and purified tumor-associated proteins or fragments thereof can comprise: MAGEC2 or a fragment thereof, TTC1 or a fragment thereof, JAKMIP2 or a fragment thereof, TXDNC2 or a fragment thereof, SULT2B1 or a fragment thereof, and NOL3 or a fragment thereof. In some embodiments, the one or more isolated and purified tumor-associated proteins or fragments thereof can comprise: MAGEC2 or a fragment thereof, TRIM33 or a fragment thereof, TXDNC2 or a fragment thereof, BCL7A or a fragment thereof, SPN or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and FIGNL2 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, N4BP1 or a fragment thereof, and TRIM33 or a fragment thereof. In some embodiments, the one or more isolated and purified tumor-associated proteins or fragments thereof can comprise: ACPP or a fragment thereof, RPLP1 or a fragment thereof, TOM1L2 or a fragment thereof, C14ORF37 or a fragment thereof, TXDNC2 or a fragment thereof, and FIGNL2 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, RPLP1 or a fragment thereof, and HRAS or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MTERF4 or a fragment thereof, KCNRG or a fragment thereof, and SSBP4 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MAGEA8 or a fragment thereof, and SSBP4 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, DDX4 or a fragment thereof, and RALGDS or a fragment thereof. In some embodiments, the one or more isolated and purified tumor-associated proteins or fragments thereof can comprise: ACPP or a fragment thereof, DCLK1 or a fragment thereof, SS18 or a fragment thereof, TOM1L2 or a fragment thereof, SPN or a fragment thereof, and NOL3 or a fragment thereof. In some embodiments, the one or more isolated and purified tumor-associated proteins or fragments thereof can comprise: ACPP or a fragment thereof, DCLK1 or a fragment thereof, TOM1L2 or a fragment thereof, TXDNC2 or a fragment thereof, FIGNL2 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and NUDT14 or a fragment thereof. In some embodiments, the one or more isolated and purified tumor-associated proteins or fragments thereof can comprise: MAGEC2 or a fragment thereof, TEX264 or a fragment thereof, TTC1 or a fragment thereof, JAKMIP2 or a fragment thereof, TXDNC2 or a fragment thereof, and NOL3 or a fragment thereof. In some embodiments, the one or more isolated and purified tumor-associated proteins or fragments thereof can comprise: ACPP or a fragment thereof, DCLK1 or a fragment thereof, RPLP1 or a fragment thereof, TOM1L2 or a fragment thereof, C14ORF37 or a fragment thereof, and JAKMIP2 or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, MAGEC2 or a fragment thereof, and MTERF4 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MAGEA8 or a fragment thereof, and RPLP1 or a fragment thereof. In some embodiments, the one or more isolated and purified tumor-associated proteins or fragments thereof can comprise: MTERF4 or a fragment thereof, TXDNC2 or a fragment thereof, BCL7A or a fragment thereof, FIGNL2 or a fragment thereof, RALGDS or a fragment thereof, and NOL3 or a fragment thereof. In some embodiments, the one or more isolated and purified tumor-associated proteins or fragments thereof can comprise: MAGEC2 or a fragment thereof, C14ORF37 or a fragment thereof, TXDNC2 or a fragment thereof, SULT2B1 or a fragment thereof, DCLK1 or a fragment thereof, and NOL3 or a fragment thereof. In some embodiments, the one or more isolated and purified tumor-associated proteins or fragments thereof can comprise: ACPP or a fragment thereof, DCLK1 or a fragment thereof, RPLP1 or a fragment thereof, SS18 or a fragment thereof, TOM1L2 or a fragment thereof, and FIGNL2 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, N4BP1 or a fragment thereof, and SSBP4 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, KCNRG or a fragment thereof, and RPLP1 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MTERF4 or a fragment thereof, DDX4 or a fragment thereof, and YP021 or a fragment thereof. In some embodiments, the one or more isolated and purified tumor-associated proteins or fragments thereof can comprise: MAGEC2 or a fragment thereof, SSBP4 or a fragment thereof, JAKMIP2 or a fragment thereof, TXDNC2 or a fragment thereof, SULT2B1 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, YP021 or a fragment thereof, RPLP1 or a fragment thereof, and SSBP4 or a fragment thereof. In some embodiments, the one or more isolated and purified tumor-associated proteins or fragments thereof can comprise: MTERF4 or a fragment thereof, RPLP1 or a fragment thereof, TOM1L2 or a fragment thereof, C14ORF37 or a fragment thereof, TTC1 or a fragment thereof, and NOL3 or a fragment thereof. In some embodiments, the one or more isolated and purified tumor-associated proteins or fragments thereof can comprise: MTERF4 or a fragment thereof, MAGEA8 or a fragment thereof, MAGEC2 or a fragment thereof, TOM1L2 or a fragment thereof, JAKMIP2 or a fragment thereof, and NOL3 or a fragment thereof. In some embodiments, the one or more isolated and purified tumor-associated proteins or fragments thereof can comprise: MAGEC2 or a fragment thereof, TEX264 or a fragment thereof, TXDNC2 or a fragment thereof, BCL7A or a fragment thereof, SPN or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, KCNRG or a fragment thereof, and SSBP4 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and SULT2B1 or a fragment thereof. In some embodiments, the one or more isolated and purified tumor-associated proteins or fragments thereof can comprise: ACPP or a fragment thereof, DCLK1 or a fragment thereof, SS18 or a fragment thereof, TOM1L2 or a fragment thereof, RALGDS or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, KCNRG or a fragment thereof, RPLP1 or a fragment thereof, and SSBP4 or a fragment thereof. In some embodiments, the one or more isolated and purified tumor-associated proteins or fragments thereof can comprise: MTERF4 or a fragment thereof, MAGEA8 or a fragment thereof, MAGEC2 or a fragment thereof, BCL7A or a fragment thereof, FIGNL2 or a fragment thereof, and NOL3 or a fragment thereof. In some embodiments, the one or more isolated and purified tumor-associated proteins or fragments thereof can comprise: MAGEC2 or a fragment thereof, TEX264 or a fragment thereof, JAKMIP2 or a fragment thereof, TXDNC2 or a fragment thereof, SULT2B1 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, APH1A or a fragment thereof, and RPLP1 or a fragment thereof. In some embodiments, the one or more isolated and purified tumor-associated proteins or fragments thereof can comprise: CTAG1A or a fragment thereof, TOM1L2 or a fragment thereof, TXDNC2 or a fragment thereof, SULT2B1 or a fragment thereof, SPN or a fragment thereof, and NOL3 or a fragment thereof. In some embodiments, the one or more isolated and purified tumor-associated proteins or fragments thereof can comprise: ACPP or a fragment thereof, RPLP1 or a fragment thereof, SS18 or a fragment thereof, TOM1L2 or a fragment thereof, C14ORF37 or a fragment thereof, and NOL3 or a fragment thereof. In some embodiments, the one or more isolated and purified tumor-associated proteins or fragments thereof can comprise: TRIM33 or a fragment thereof, BCL7A or a fragment thereof, CTAG1A or a fragment thereof, MTERF4 or a fragment thereof, YP021 or a fragment thereof, and C1QTNF1 or a fragment thereof. In some embodiments, the one or more isolated and purified tumor-associated proteins or fragments thereof can comprise: ACPP or a fragment thereof, RPLP1 or a fragment thereof, TOM1L2 or a fragment thereof, TXDNC2 or a fragment thereof, SPN or a fragment thereof, and NOL3 or a fragment thereof. In some embodiments, the one or more isolated and purified tumor-associated proteins or fragments thereof can comprise: MAGEC2 or a fragment thereof, TEX264 or a fragment thereof, TRIM33 or a fragment thereof, TXDNC2 or a fragment thereof, SULT2B1 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, APH1A or a fragment thereof, RPLP1 or a fragment thereof, and SSBP4 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MTERF4 or a fragment thereof, APH1A or a fragment thereof, and SSBP4 or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, and SSBP4 or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, and SSBP4 or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, APH1A or a fragment thereof, and SSBP4 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, and NUDT14 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, NUDT14 or a fragment thereof, and SSBP4 or a fragment thereof. In some embodiments, the one or more isolated and purified tumor-associated proteins or fragments thereof can comprise: MAGEC2 or a fragment thereof, TXDNC2 or a fragment thereof, BCL7A or a fragment thereof, FIGNL2 or a fragment thereof, SPN or a fragment thereof, and NOL3 or a fragment thereof. In some embodiments, the one or more isolated and purified tumor-associated proteins or fragments thereof can comprise: ACPP or a fragment thereof, APH1A or a fragment thereof, DCLK1 or a fragment thereof, TOM1L2 or a fragment thereof, JAKMIP2 or a fragment thereof, and NOL3 or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, and C1QTNF1 or a fragment thereof. In some embodiments, the one or more isolated and purified tumor-associated proteins or fragments thereof can comprise: ACPP or a fragment thereof, NUDT14 or a fragment thereof, DCLK1 or a fragment thereof, RPLP1 or a fragment thereof, SS18 or a fragment thereof, and NOL3 or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, MTERF4 or a fragment thereof, and NUMBL or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and RPLP1 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, NUDT14 or a fragment thereof, and NOL3 or a fragment thereof. In some embodiments, the one or more isolated and purified tumor-associated proteins or fragments thereof can comprise: BCL7A or a fragment thereof, CTAG1A or a fragment thereof, MAGEC2 or a fragment thereof, MTERF4 or a fragment thereof, DDX4 or a fragment thereof, and RALGDS or a fragment thereof. In some embodiments, the one or more isolated and purified tumor-associated proteins or fragments thereof can comprise: MAGEC2 or a fragment thereof, TOM1L2- or a fragment thereof, JAKMIP2 or a fragment thereof, SULT2B1 or a fragment thereof, DCLK1 or a fragment thereof, and NOL3 or a fragment thereof. In some embodiments, the one or more isolated and purified tumor-associated proteins or fragments thereof can comprise: ACPP or a fragment thereof, DCLK1 or a fragment thereof, RPLP1 or a fragment thereof, SS18 or a fragment thereof, JAKMIP2 or a fragment thereof, and NOL3 or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, RPLP1 or a fragment thereof, and C1QTNF1 or a fragment thereof. In some embodiments, the one or more isolated and purified tumor-associated proteins or fragments thereof can comprise: TRIM33 or a fragment thereof, SSBP4 or a fragment thereof, TP53 or a fragment thereof, NSG1 or a fragment thereof, and CTAG1A or a fragment thereof. In some embodiments, the one or more isolated and purified tumor-associated proteins or fragments thereof can comprise: TRIM33 or a fragment thereof, MTERF4 or a fragment thereof, TP53 or a fragment thereof, MAGEC2 or a fragment thereof, and CTAG1A or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, N4BP1 or a fragment thereof, and RALGDS or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, RPLP1 or a fragment thereof, and SSBP4 or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, MTERF4 or a fragment thereof, and SSBP4 or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, C1QTNF1 or a fragment thereof, and FIGNL2 or a fragment thereof. In some embodiments, the one or more isolated and purified tumor-associated proteins or fragments thereof can comprise: ACPP or a fragment thereof, DCLK1 or a fragment thereof, SS18 or a fragment thereof, TOM1L2 or a fragment thereof, JAKMIP2 or a fragment thereof, and NOL3 or a fragment thereof. In some embodiments, the one or more isolated and purified tumor-associated proteins or fragments thereof can comprise: ACPP or a fragment thereof, NUDT14 or a fragment thereof, DCLK1 or a fragment thereof, RPLP1 or a fragment thereof, SS18 or a fragment thereof, and TOM1L2 or a fragment thereof. In some embodiments, the one or more isolated and purified tumor-associated proteins or fragments thereof can comprise: ACPP or a fragment thereof, NUDT14 or a fragment thereof, DCLK1 or a fragment thereof, RPLP1 or a fragment thereof, TOM1L2 or a fragment thereof, and JAKMIP2 or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, MTERF4 or a fragment thereof, and RALGDS or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, MTERF4 or a fragment thereof, and C1QTNF1 or a fragment thereof. In some embodiments, the one or more isolated and purified tumor-associated proteins or fragments thereof can comprise: ACPP or a fragment thereof, DCLK1 or a fragment thereof, TOM1L2 or a fragment thereof, FIGNL2 or a fragment thereof, SPN or a fragment thereof, and NOL3 or a fragment thereof. In some embodiments, the one or more isolated and purified tumor-associated proteins or fragments thereof can comprise: ACPP or a fragment thereof, DCLK1 or a fragment thereof, RPLP1 or a fragment thereof, TOM1L2 or a fragment thereof, FIGNL2 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, NSG1 or a fragment thereof, and NUMBL or a fragment thereof. In some embodiments, the one or more isolated and purified tumor-associated proteins or fragments thereof can comprise: ACPP or a fragment thereof, DCLK1 or a fragment thereof, RPLP1 or a fragment thereof, SS18 or a fragment thereof, TOM1L2 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and RALGDS or a fragment thereof. In some embodiments, the one or more isolated and purified tumor-associated proteins or fragments thereof can comprise: BCL7A or a fragment thereof, CTAG1A or a fragment thereof, MAGEC2 or a fragment thereof, MTERF4 or a fragment thereof, NUDT14 or a fragment thereof, and NUMBL or a fragment thereof.

In some embodiments, the one or more isolated and purified tumor-associated proteins or fragments thereof can comprise: ACPP or a fragment thereof, NUDT14 or a fragment thereof, DCLK1 or a fragment thereof, SS18 or a fragment thereof, TOM1L2 or a fragment thereof, and FIGNL2 or a fragment thereof. In some embodiments, the one or more isolated and purified tumor-associated proteins or fragments thereof can comprise: MAGEC2 or a fragment thereof, C14ORF37 or a fragment thereof, PIP4K2C or a fragment thereof, SULT2B1 or a fragment thereof, DCLK1 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, NSG1 or a fragment thereof, and RALGDS or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, and RPLP1 or a fragment thereof. In some embodiments, the one or more isolated and purified tumor-associated proteins or fragments thereof can comprise: ACPP or a fragment thereof, NUDT14 or a fragment thereof, DCLK1 or a fragment thereof, SS18 or a fragment thereof, FIGNL2 or a fragment thereof, and NOL3 or a fragment thereof. In some embodiments, the one or more isolated and purified tumor-associated proteins or fragments thereof can comprise: BCL7A or a fragment thereof, CTAG1A or a fragment thereof, MAGEC2 or a fragment thereof, MTERF4 or a fragment thereof, SS18 or a fragment thereof, and NUMBL or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MAGEA8 or a fragment thereof, and TRIM33 or a fragment thereof. In some embodiments, the one or more isolated and purified tumor-associated proteins or fragments thereof can comprise: ACPP or a fragment thereof, NUDT14 or a fragment thereof, DCLK1 or a fragment thereof, RPLP1 or a fragment thereof, JAKMIP2 or a fragment thereof, and NOL3 or a fragment thereof. In some embodiments, the one or more isolated and purified tumor-associated proteins or fragments thereof can comprise: BCL7A-A or a fragment thereof, DDX4 or a fragment thereof, CTAG1A or a fragment thereof, MAGEC2 or a fragment thereof, MTERF4 or a fragment thereof, and SSBP4 or a fragment thereof. In some embodiments, the one or more isolated and purified tumor-associated proteins or fragments thereof can comprise: MAGEC2 or a fragment thereof, TXDNC2 or a fragment thereof, SULT2B1 or a fragment thereof, SPN or a fragment thereof, DCLK1 or a fragment thereof, and NOL3 or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, MTERF4 or a fragment thereof, and HRAS or a fragment thereof. In some embodiments, the one or more isolated and purified tumor-associated proteins or fragments thereof can comprise: MAGEC2 or a fragment thereof, TOM1L2 or a fragment thereof, TTC1 or a fragment thereof, JAKMIP2 or a fragment thereof, DCLK1 or a fragment thereof, and NOL3 or a fragment thereof. In some embodiments, the one or more isolated and purified tumor-associated proteins or fragments thereof can comprise: ACPP or a fragment thereof, RPLP1 or a fragment thereof, SS18 or a fragment thereof, TOM1L2 or a fragment thereof, C14ORF37 or a fragment thereof, and FIGNL2 or a fragment thereof. In some embodiments, the one or more isolated and purified tumor-associated proteins or fragments thereof can comprise: MAGEC2 or a fragment thereof, TXDNC2 or a fragment thereof, FIGNL2 or a fragment thereof, SPN or a fragment thereof, DCLK1 or a fragment thereof, and NOL3 or a fragment thereof. In some embodiments, the one or more isolated and purified tumor-associated proteins or fragments thereof can comprise: MAGEC2 or a fragment thereof, TXDNC2 or a fragment thereof, FIGNL2 or a fragment thereof, SULT2B1 or a fragment thereof, DCLK1 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, NUDT14 or a fragment thereof, and FIGNL2 or a fragment thereof. In some embodiments, the one or more isolated and purified tumor-associated proteins or fragments thereof can comprise: TRIM33 or a fragment thereof, BCL7A or a fragment thereof, CTAG1A or a fragment thereof, MAGEC2 or a fragment thereof, MTERF4 or a fragment thereof, and TRIM33 or a fragment thereof. In some embodiments, the one or more isolated and purified tumor-associated proteins or fragments thereof can comprise: MAGEC2 or a fragment thereof, TTC1 or a fragment thereof, JAKMIP2 or a fragment thereof, TXDNC2 or a fragment thereof, DCLK1 or a fragment thereof, and NOL3 or a fragment thereof. In some embodiments, the one or more isolated and purified tumor-associated proteins or fragments thereof can comprise: TRIM33 or a fragment thereof, BCL7A or a fragment thereof, CTAG1A or a fragment thereof, MAGEC2 or a fragment thereof, and C1QTNF1 or a fragment thereof. In some embodiments, the one or more isolated and purified tumor-associated proteins or fragments thereof can comprise: MTERF4 or a fragment thereof, TOM1L2 or a fragment thereof, TXDNC2 or a fragment thereof, BCL7A or a fragment thereof, SULT2B1 or a fragment thereof, and NOL3 or a fragment thereof. In some embodiments, the one or more isolated and purified tumor-associated proteins or fragments thereof can comprise: MAGEC2 or a fragment thereof, RPLP1 or a fragment thereof, TOM1L2 or a fragment thereof, JAKMIP2 or a fragment thereof, DCLK1 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and NUMBL or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, TOM1L2 or a fragment thereof, and SSBP4 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, SSBP4 or a fragment thereof, and TRIM33 or a fragment thereof. In some embodiments, the one or more isolated and purified tumor-associated proteins or fragments thereof can comprise: ACPP or a fragment thereof, NUDT14 or a fragment thereof, DCLK1 or a fragment thereof, RPLP1 or a fragment thereof, TOM1L2 or a fragment thereof, and NOL3 or a fragment thereof. In some embodiments, the one or more isolated and purified tumor-associated proteins or fragments thereof can comprise: MTERF4 or a fragment thereof, RPLP1 or a fragment thereof, TOM1L2 or a fragment thereof, TTC1 or a fragment thereof, JAKMIP2 or a fragment thereof, and NOL3 or a fragment thereof. In some embodiments, the one or more isolated and purified tumor-associated proteins or fragments thereof can comprise: MAGEC2 or a fragment thereof, TRIM33 or a fragment thereof, TXDNC2 or a fragment thereof, FIGNL2 or a fragment thereof, SPN or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, and DDX4 or a fragment thereof. In some embodiments, the one or more isolated and purified tumor-associated proteins or fragments thereof can comprise: MTERF4 or a fragment thereof, NSG1 or a fragment thereof, TEX264 or a fragment thereof, TTC1 or a fragment thereof, JAKMIP2 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MTERF4 or a fragment thereof, YP021 or a fragment thereof, and SSBP4 or a fragment thereof. In some embodiments, the one or more isolated and purified tumor-associated proteins or fragments thereof can comprise: MAGEC2 or a fragment thereof, SSBP4 or a fragment thereof, TXDNC2 or a fragment thereof, FIGNL2 or a fragment thereof, SPN or a fragment thereof, and NOL3 or a fragment thereof. In some embodiments, the one or more isolated and purified tumor-associated proteins or fragments thereof can comprise: ACPP or a fragment thereof, DCLK1 or a fragment thereof, TOM1L2 or a fragment thereof, TTC1 or a fragment thereof, JAKMIP2 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, NUDT14 or a fragment thereof, and RALGDS or a fragment thereof. In some embodiments, the one or more isolated and purified tumor-associated proteins or fragments thereof can comprise: ACPP or a fragment thereof, RPLP1 or a fragment thereof, TOM1L2 or a fragment thereof, JAKMIP2 or a fragment thereof, FIGNL2 or a fragment thereof, and NOL3 or a fragment thereof. In some embodiments, the one or more isolated and purified tumor-associated proteins or fragments thereof can comprise: MAGEC2 or a fragment thereof, TEX264 or a fragment thereof, TRIM33 or a fragment thereof, TXDNC2 or a fragment thereof, SPN or a fragment thereof, and NOL3 or a fragment thereof. In some embodiments, the one or more isolated and purified tumor-associated proteins or fragments thereof can comprise: ACPP or a fragment thereof, RPLP1 or a fragment thereof, SS18 or a fragment thereof, TOM1L2 or a fragment thereof, JAKMIP2 or a fragment thereof, and NOL3 or a fragment thereof. In some embodiments, the one or more isolated and purified tumor-associated proteins or fragments thereof can comprise: ACPP or a fragment thereof, DCLK1 or a fragment thereof, SS18 or a fragment thereof, TOM1L2 or a fragment thereof, TTC1 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, and N4BP1 or a fragment thereof. In some embodiments, the one or more isolated and purified tumor-associated proteins or fragments thereof can comprise: MAGEC2 or a fragment thereof, NSG1 or a fragment thereof, TEX264 or a fragment thereof, TRIM33 or a fragment thereof, JAKMIP2 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEA8 or a fragment thereof, RPLP1 or a fragment thereof, and SSBP4 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, and MAGEA8 or a fragment thereof. In some embodiments, the one or more isolated and purified tumor-associated proteins or fragments thereof can comprise: ACPP or a fragment thereof, DCLK1 or a fragment thereof, SS18 or a fragment thereof, TOM1L2 or a fragment thereof, TXDNC2 or a fragment thereof, and NOL3 or a fragment thereof. In some embodiments, the one or more isolated and purified tumor-associated proteins or fragments thereof can comprise: ACPP or a fragment thereof, DCLK1 or a fragment thereof, SS18 or a fragment thereof, FIGNL2 or a fragment thereof, SPN or a fragment thereof, and NOL3 or a fragment thereof. In some embodiments, the one or more isolated and purified tumor-associated proteins or fragments thereof can comprise: MAGEC2 or a fragment thereof, TXDNC2 or a fragment thereof, BCL7A or a fragment thereof, SPN or a fragment thereof, DCLK1 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MTERF4 or a fragment thereof, TP53 or a fragment thereof, and KCNRG or a fragment thereof. In some embodiments, the one or more isolated and purified tumor-associated proteins or fragments thereof can comprise: ACPP or a fragment thereof, DCLK1 or a fragment thereof, TOM1L2 or a fragment thereof, JAKMIP2 or a fragment thereof, FIGNL2 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, RPLP1 or a fragment thereof, and TRIM33 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and NSG1 or a fragment thereof. In some embodiments, the one or more isolated and purified tumor-associated proteins or fragments thereof can comprise: ACPP or a fragment thereof, NUDT14 or a fragment thereof, DCLK1 or a fragment thereof, TOM1L2 or a fragment thereof, TXDNC2 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MTERF4 or a fragment thereof, ACPP or a fragment thereof, and C1QTNF1 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, and MTERF4 or a fragment thereof. In some embodiments, the one or more isolated and purified tumor-associated proteins or fragments thereof can comprise: MAGEC2 or a fragment thereof, JAKMIP2 or a fragment thereof, TXDNC2 or a fragment thereof, SULT2B1 or a fragment thereof, DCLK1 or a fragment thereof, and NOL3 or a fragment thereof. In some embodiments, the one or more isolated and purified tumor-associated proteins or fragments thereof can comprise: MAGEC2 or a fragment thereof, JAKMIP2 or a fragment thereof, TXDNC2 or a fragment thereof, FIGNL2 or a fragment thereof, SULT2B1 or a fragment thereof, and NOL3 or a fragment thereof. In some embodiments, the one or more isolated and purified tumor-associated proteins or fragments thereof can comprise: ACPP or a fragment thereof, APH1A or a fragment thereof, DCLK1 or a fragment thereof, SS18 or a fragment thereof, TOM1L2 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MTERF4 or a fragment thereof, RPLP1 or a fragment thereof, and SSBP4 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and N4BP1 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and TRIM33 or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, KCNRG or a fragment thereof, and SSBP4 or a fragment thereof. In some embodiments, the one or more isolated and purified tumor-associated proteins or fragments thereof can comprise: MAGEC2 or a fragment thereof, TXDNC2 or a fragment thereof, FIGNL2 or a fragment thereof, SULT2B1 or a fragment thereof, SPN or a fragment thereof, and NOL3 or a fragment thereof. In some embodiments, the one or more isolated and purified tumor-associated proteins or fragments thereof can comprise: ACPP or a fragment thereof, DCLK1 or a fragment thereof, RPLP1 or a fragment thereof, TOM1L2 or a fragment thereof, JAKMIP2-A or a fragment thereof, NOL3-A or a fragment thereof. In some embodiments, the one or more isolated and purified tumor-associated proteins or fragments thereof can comprise: ACPP or a fragment thereof, DCLK1 or a fragment thereof, SS18 or a fragment thereof, TOM1L2 or a fragment thereof, DCLK1 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, APH1A or a fragment thereof, and SSBP4 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, NUDT14 or a fragment thereof, and TRIM33 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, KCNRG or a fragment thereof, and N4BP1 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, N4BP1 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, YP021 or a fragment thereof, SSBP4 or a fragment thereof. In some embodiments, the one or more isolated and purified tumor-associated proteins or fragments thereof can comprise: MAGEC2 or a fragment thereof, NSG1 or a fragment thereof, TEX264 or a fragment thereof, JAKMIP2 or a fragment thereof, SULT2B1 or a fragment thereof, and NOL3 or a fragment thereof. In some embodiments, the one or more isolated and purified tumor-associated proteins or fragments thereof can comprise: ACPP or a fragment thereof, NUDT14 or a fragment thereof, SS18 or a fragment thereof, TOM1L2 or a fragment thereof, FIGNL2 or a fragment thereof, and NOL3 or a fragment thereof. In some embodiments, the one or more isolated and purified tumor-associated proteins or fragments thereof can comprise: MTERF4 or a fragment thereof, RPLP1 or a fragment thereof, SS18 or a fragment thereof, TOM1L2 or a fragment thereof, JAKMIP2 or a fragment thereof, and NOL3 or a fragment thereof. In some embodiments, the one or more isolated and purified tumor-associated proteins or fragments thereof can comprise: MAGEC2 or a fragment thereof, TXDNC2 or a fragment thereof, BCL7A or a fragment thereof, SULT2B1 or a fragment thereof, DCLK1 or a fragment thereof, and NOL3 or a fragment thereof. In some embodiments, the one or more isolated and purified tumor-associated proteins or fragments thereof can comprise: ACPP or a fragment thereof, DCLK1 or a fragment thereof, SS18 or a fragment thereof, TOM1L2 or a fragment thereof, C14ORF37 or a fragment thereof, and FIGNL2 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and SULT2B1 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, RPLP1 or a fragment thereof, and RALGDS or a fragment thereof. In some embodiments, the one or more isolated and purified tumor-associated proteins or fragments thereof can comprise: ACPP or a fragment thereof, DCLK1 or a fragment thereof, RPLP1 or a fragment thereof, SS18 or a fragment thereof, TOM1L2 or a fragment thereof, and JAKMIP2 or a fragment thereof. In some embodiments, the one or more isolated and purified tumor-associated proteins or fragments thereof can comprise: DDX4 or a fragment thereof, CTAG1A or a fragment thereof, MTERF4 or a fragment thereof, YP021 or a fragment thereof, RALGDS or a fragment thereof, and PIP4K2C or a fragment thereof. In some embodiments, the one or more isolated and purified tumor-associated proteins or fragments thereof can comprise: MTERF4 or a fragment thereof, TOM1L2 or a fragment thereof, TTC1 or a fragment thereof, JAKMIP2 or a fragment thereof, FIGNL2 or a fragment thereof, and NOL3 or a fragment thereof. In some embodiments, the one or more isolated and purified tumor-associated proteins or fragments thereof can comprise: ACPP or a fragment thereof, DCLK1 or a fragment thereof, SS18 or a fragment thereof, TOM1L2 or a fragment thereof, FIGNL2 or a fragment thereof, and SPN or a fragment thereof. In some embodiments, the one or more isolated and purified tumor-associated proteins or fragments thereof can comprise: CTAG1A or a fragment thereof, TOM1L2 or a fragment thereof, TXDNC2 or a fragment thereof, SULT2B1 or a fragment thereof, DCLK1 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and KCNRG or a fragment thereof. In some embodiments, the one or more isolated and purified tumor-associated proteins or fragments thereof can comprise: MTERF4 or a fragment thereof, TXDNC2 or a fragment thereof, BCL7A or a fragment thereof, FIGNL2 or a fragment thereof, SULT2B1 or a fragment thereof, and NOL3 or a fragment thereof. In some embodiments, the one or more isolated and purified tumor-associated proteins or fragments thereof can comprise: ACPP or a fragment thereof, NUDT14 or a fragment thereof, DCLK1 or a fragment thereof, SS18 or a fragment thereof, TOM1L2 or a fragment thereof, and NOL3 or a fragment thereof. In some embodiments, the one or more isolated and purified tumor-associated proteins or fragments thereof can comprise: TRIM33 or a fragment thereof, BCL7A or a fragment thereof, CTAG1A or a fragment thereof, MAGEC2 or a fragment thereof, MTERF4 or a fragment thereof, and HRAS or a fragment thereof. In some embodiments, the one or more isolated and purified tumor-associated proteins or fragments thereof can comprise: ACPP or a fragment thereof, DCLK1 or a fragment thereof, SS18 or a fragment thereof, TOM1L2 or a fragment thereof, FIGNL2 or a fragment thereof, and NOL3 or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, C1QTNF1 or a fragment thereof, and SSBP4 or a fragment thereof. In some embodiments, the one or more isolated and purified tumor-associated proteins or fragments thereof can comprise: CTAG1A or a fragment thereof, TXDNC2 or a fragment thereof, BCL7A or a fragment thereof, SULT2B1 or a fragment thereof, DCLK1 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, APH1A or a fragment thereof, and N4BP1 or a fragment thereof. In some embodiments, the one or more isolated and purified tumor-associated proteins or fragments thereof can comprise: MTERF4 or a fragment thereof, RPLP1 or a fragment thereof, SS18 or a fragment thereof, TOM1L2 or a fragment thereof, TTC1 or a fragment thereof, and NOL3 or a fragment thereof. In some embodiments, the one or more isolated and purified tumor-associated proteins or fragments thereof can comprise: ACPP or a fragment thereof, NUDT14 or a fragment thereof, DCLK1 or a fragment thereof, TOM1L2 or a fragment thereof, FIGNL2 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, NUDT14 or a fragment thereof, and HRAS or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MAGEA8 or a fragment thereof, and RALGDS or a fragment thereof. In some embodiments, the one or more isolated and purified tumor-associated proteins or fragments thereof can comprise: MTERF4 or a fragment thereof, RPLP1 or a fragment thereof, TOM1L2 or a fragment thereof, JAKMIP2 or a fragment thereof, SULT2B1 or a fragment thereof, and NOL3 or a fragment thereof. In some embodiments, the one or more isolated and purified tumor-associated proteins or fragments thereof can comprise: MAGEC2 or a fragment thereof, TRIM33 or a fragment thereof, JAKMIP2 or a fragment thereof, TXDNC2 or a fragment thereof, SULT2B1 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and NOL3 or a fragment thereof. In some embodiments, the one or more isolated and purified tumor-associated proteins or fragments thereof can comprise: TRIM33 or a fragment thereof, BCL7A or a fragment thereof, CTAG1A or a fragment thereof, MAGEC2 or a fragment thereof, MTERF4 or a fragment thereof, and C1QTNF1 or a fragment thereof. In some embodiments, the one or more isolated and purified tumor-associated proteins or fragments thereof can comprise: MTERF4 or a fragment thereof, RPLP1 or a fragment thereof, SS18 or a fragment thereof, TOM1L2 or a fragment thereof, SPN or a fragment thereof, and NOL3 or a fragment thereof. In some embodiments, the one or more isolated and purified tumor-associated proteins or fragments thereof can comprise: TRIM33 or a fragment thereof, BCL7A or a fragment thereof, CTAG1A or a fragment thereof, MAGEC2 or a fragment thereof, MTERF4 or a fragment thereof, and SSBP4 or a fragment thereof. In some embodiments, the one or more isolated and purified tumor-associated proteins or fragments thereof can comprise: TRIM33 or a fragment thereof, MAGEA8 or a fragment thereof, MAGEC2 or a fragment thereof, TOM1L2 or a fragment thereof, JAKMIP2 or a fragment thereof, and NOL3 or a fragment thereof. In some embodiments, the one or more isolated and purified tumor-associated proteins or fragments thereof can comprise: ACPP or a fragment thereof, DCLK1 or a fragment thereof, RPLP1 or a fragment thereof, SS18 or a fragment thereof, TOM1L2 or a fragment thereof, and C14ORF37 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and APH1A or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MTERF4 or a fragment thereof, YP021 or a fragment thereof, and PIP4K2C or a fragment thereof. In some embodiments, the one or more isolated and purified tumor-associated proteins or fragments thereof can comprise: MAGEC2 or a fragment thereof, TOM1L2 or a fragment thereof, JAKMIP2 or a fragment thereof, TXDNC2 or a fragment thereof, SULT2B1 or a fragment thereof, and NOL3 or a fragment thereof. In some embodiments, the one or more isolated and purified tumor-associated proteins or fragments thereof can comprise: ACPP or a fragment thereof, NUDT14 or a fragment thereof, DCLK1 or a fragment thereof, TOM1L2 or a fragment thereof, JAKMIP2 or a fragment thereof, and NOL3 or a fragment thereof. In some embodiments, the one or more isolated and purified tumor-associated proteins or fragments thereof can comprise: ACPP or a fragment thereof, RPLP1 or a fragment thereof, SS18 or a fragment thereof, TOM1L2 or a fragment thereof, FIGNL2 or a fragment thereof, and NOL3 or a fragment thereof. In some embodiments, the one or more isolated and purified tumor-associated proteins or fragments thereof can comprise: ACPP or a fragment thereof, RPLP1 or a fragment thereof, TOM1L2 or a fragment thereof, C14ORF37 or a fragment thereof, JAKMIP2 or a fragment thereof, and NOL3 or a fragment thereof. In some embodiments, the one or more isolated and purified tumor-associated proteins or fragments thereof can comprise: ACPP or a fragment thereof, RPLP1 or a fragment thereof, TOM1L2 or a fragment thereof, TTC1 or a fragment thereof, JAKMIP2 or a fragment thereof, and NOL3 or a fragment thereof. In some embodiments, the one or more isolated and purified tumor-associated proteins or fragments thereof can comprise: ACPP or a fragment thereof, RPLP1 or a fragment thereof, SS18 or a fragment thereof, TOM1L2 or a fragment thereof, SPN or a fragment thereof, and NOL3 or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, MTERF4 or a fragment thereof, and APH1A or a fragment thereof. In some embodiments, the one or more isolated and purified tumor-associated proteins or fragments thereof can comprise: MAGEC2 or a fragment thereof, JAKMIP2 or a fragment thereof, TXDNC2 or a fragment thereof, SULT2B1 or a fragment thereof, SPN or a fragment thereof, and NOL3 or a fragment thereof. In some embodiments, the one or more isolated and purified tumor-associated proteins or fragments thereof can comprise: TRIM33 or a fragment thereof, BCL7A or a fragment thereof, DDX4 or a fragment thereof, CTAG1A or a fragment thereof, MTERF4 or a fragment thereof, and KCNRG or a fragment thereof. In some embodiments, the one or more isolated and purified tumor-associated proteins or fragments thereof can comprise: ACPP or a fragment thereof, RPLP1 or a fragment thereof, TOM1L2 or a fragment thereof, FIGNL2 or a fragment thereof, SPN or a fragment thereof, NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and RALGDS or a fragment thereof. In some embodiments, the one or more isolated and purified tumor-associated proteins or fragments thereof can comprise: MTERF4 or a fragment thereof, TOM1L2 or a fragment thereof, JAKMIP2 or a fragment thereof, TXDNC2 or a fragment thereof, SULT2B1 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, N4BP1 or a fragment thereof, and HRAS or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MTERF4 or a fragment thereof, APH1A or a fragment thereof, and RALGDS or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, MTERF4 or a fragment thereof, and HRAS or a fragment thereof. In some embodiments, the one or more isolated and purified tumor-associated proteins or fragments thereof can comprise: MTERF4 or a fragment thereof, TOM1L2 or a fragment thereof, TTC1 or a fragment thereof, JAKMIP2 or a fragment thereof, TXDNC2 or a fragment thereof, and NOL3 or a fragment thereof. In some embodiments, the one or more isolated and purified tumor-associated proteins or fragments thereof can comprise: BCL7A or a fragment thereof, DDX4 or a fragment thereof, CTAG1A or a fragment thereof, MAGEC2 or a fragment thereof, SSBP4 or a fragment thereof, and RALGDS or a fragment thereof. In some embodiments, the one or more isolated and purified tumor-associated proteins or fragments thereof can comprise: MAGEC2 or a fragment thereof, TEX264 or a fragment thereof, TRIM33 or a fragment thereof, JAKMIP2 or a fragment thereof, SULT2B1 or a fragment thereof, and NOL3 or a fragment thereof.

In some embodiments, the one or more isolated and purified tumor-associated proteins can comprise polypeptides having 70%, 75%, 80%, 85%, 90%, 95%, 96%, 97%, 98%, 99%, 99.5%, or 100% identity to the polypeptide sequences of the proteins listed in their respective combinations in Table 7. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO:137; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 133. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 134; and a polypeptide having 70%, 75%, 80%, 85%, 90%, 95%, 96%, 97%, 98%, 99%, 99.5% or 100% sequence identity to the polypeptide sequence of SEQ ID NO: 130. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO:140; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 146; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:124; and a polypeptide having 70%, 75%, 80%, 85%, 90%, 95%, 96%, 97%, 98%, 99%, 99.5% or 100% sequence identity to the polypeptide sequence of SEQ ID NO:131. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO: 128; and a polypeptide having 70%, 75%, 80%, 85%, 90%, 95%, 96%, 97%, 98%, 99%, 99.5% or 100% sequence identity to the polypeptide sequence of SEQ ID NO: 132. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 135; and a polypeptide having 70%, 75%, 80%, 85%, 90%, 95%, 96%, 97%, 98%, 99%, 99.5% or 100% sequence identity to the polypeptide sequence of SEQ ID NO: 12. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:129; and a polypeptide having 70%, 75%, 80%, 85%, 90%, 95%, 96%, 97%, 98%, 99%, 99.5% or 100% sequence identity to the polypeptide sequence of SEQ ID NO:130. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:124; and a polypeptide having 70%, 75%, 80%, 85%, 90%, 95%, 96%, 97%, 98%, 99%, 99.5% or 100% sequence identity to the polypeptide sequence of SEQ ID NO: 130. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO:142; a polypeptide having 70%, 75%, 80%, 85%, 90%, 95%, 96%, 97%, 98%, 99%, 99.5% or 100% sequence identity to the polypeptide sequence of SEQ ID NO: 144; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO: 127; and a polypeptide having 70%, 75%, 80%, 85%, 90%, 95%, 96%, 97%, 98%, 99%, 99.5% or 100% sequence identity to the polypeptide sequence of SEQ ID NO: 129. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:149; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO: 142; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:126; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO:144; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 137. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 132; and a polypeptide having 70%, 75%, 80%, 85%, 90%, 95%, 96%, 97%, 98%, 99%, 99.5% or 100% sequence identity to the polypeptide sequence of SEQ ID NO: 126. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:141; a polypeptide having 70%, 75%, 80%, 85%, 90%, 95%, 96%, 97%, 98%, 99%, 99.5% or 100% sequence identity to the polypeptide sequence of SEQ ID NO:128; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 146; a polypeptide having 70%, 75%, 80%, 85%, 90%, 95%, 96%, 97%, 98%, 99%, 99.5% or 100% sequence identity to the polypeptide sequence of SEQ ID NO: 147; and a polypeptide having 70%, 75%, 80%, 85%, 90%, 95%, 96%, 97%, 98%, 99%, 99.5% or 100% sequence identity to the polypeptide sequence of SEQ ID NO: 137. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:128; and a polypeptide having 70%, 75%, 80%, 85%, 90%, 95%, 96%, 97%, 98%, 99%, 99.5% or 100% sequence identity to the polypeptide sequence of SEQ ID NO: 12. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:124; a polypeptide having 70%, 75%, 80%, 85%, 90%, 95%, 96%, 97%, 98%, 99%, 99.5% or 100% sequence identity to the polypeptide sequence of SEQ ID NO: 134; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 135; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO:122; and a polypeptide having 70%, 75%, 80%, 85%, 90%, 95%, 96%, 97%, 98%, 99%, 99.5% or 100% sequence identity to the polypeptide sequence of SEQ ID NO:131. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO:145; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO:144; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:141; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO:147; a polypeptide having 70%, 75%, 80%, 85%, 90%, 95%, 96%, 97%, 98%, 99%, 99.5% or 100% sequence identity to the polypeptide sequence of SEQ ID NO: 137; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 127. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:9; a polypeptide having 70%, 75%, 80%, 85%, 90%, 95%, 96%, 97%, 98%, 99%, 99.5% or 100% sequence identity to the polypeptide sequence of SEQ ID NO: 149; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO:147; and a polypeptide having 70%, 75%, 80%, 85%, 90%, 95%, 96%, 97%, 98%, 99%, 99.5% or 100% sequence identity to the polypeptide sequence of SEQ ID NO:138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:141; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO:143; a polypeptide having 70%, 75%, 80%, 85%, 90%, 95%, 96%, 97%, 98%, 99%, 99.5% or 100% sequence identity to the polypeptide sequence of SEQ ID NO: 146; and a polypeptide having 70%, 75%, 80%, 85%, 90%, 95%, 96%, 97%, 98%, 99%, 99.5% or 100% sequence identity to the polypeptide sequence of SEQ ID NO: 148. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; and a polypeptide having 70%, 75%, 80%, 85%, 90%, 95%, 96%, 97%, 98%, 99%, 99.5% or 100% sequence identity to the polypeptide sequence of SEQ ID NO: 124. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 135; and a polypeptide having 70%, 75%, 80%, 85%, 90%, 95%, 96%, 97%, 98%, 99%, 99.5% or 100% sequence identity to the polypeptide sequence of SEQ ID NO: 128. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO:147; a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 137; a polypeptide having 70%, 75%, 80%, 85%, 90%, 95%, 96%, 97%, 98%, 99%, 99.5% or 100% sequence identity to the polypeptide sequence of SEQ ID NO:131; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 146; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO:142; a polypeptide having 70%, 75%, 80%, 85%, 90%, 95%, 96%, 97%, 98%, 99%, 99.5% or 100% sequence identity to the polypeptide sequence of SEQ ID NO: 145; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:141; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO:140; a polypeptide having 70%, 75%, 80%, 85%, 90%, 95%, 96%, 97%, 98%, 99%, 99.5% or 100% sequence identity to the polypeptide sequence of SEQ ID NO: 143; and a polypeptide having 70%, 75%, 80%, 85%, 90%, 95%, 96%, 97%, 98%, 99%, 99.5% or 100% sequence identity to the polypeptide sequence of SEQ ID NO: 137. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 132; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:134; and a polypeptide having 70%, 75%, 80%, 85%, 90%, 95%, 96%, 97%, 98%, 99%, 99.5% or 100% sequence identity to the polypeptide sequence of SEQ ID NO: 128. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:124; a polypeptide having 70%, 75%, 80%, 85%, 90%, 95%, 96%, 97%, 98%, 99%, 99.5% or 100% sequence identity to the polypeptide sequence of SEQ ID NO: 122; and a polypeptide having 70%, 75%, 80%, 85%, 90%, 95%, 96%, 97%, 98%, 99%, 99.5% or 100% sequence identity to the polypeptide sequence of SEQ ID NO: 133. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 125; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO:147; a polypeptide having 70%, 75%, 80%, 85%, 90%, 95%, 96%, 97%, 98%, 99%, 99.5% or 100% sequence identity to the polypeptide sequence of SEQ ID NO: 142; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 133; a polypeptide having 70%, 75%, 80%, 85%, 90%, 95%, 96%, 97%, 98%, 99%, 99.5% or 100% sequence identity to the polypeptide sequence of SEQ ID NO: 128; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 146; a polypeptide having 70%, 75%, 80%, 85%, 90%, 95%, 96%, 97%, 98%, 99%, 99.5% or 100% sequence identity to the polypeptide sequence of SEQ ID NO: 149; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO: 135; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:143; a polypeptide having 70%, 75%, 80%, 85%, 90%, 95%, 96%, 97%, 98%, 99%, 99.5% or 100% sequence identity to the polypeptide sequence of SEQ ID NO: 148; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:9; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 144; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 134; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:124; and a polypeptide having 70%, 75%, 80%, 85%, 90%, 95%, 96%, 97%, 98%, 99%, 99.5% or 100% sequence identity to the polypeptide sequence of SEQ ID NO: 142. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO:143; a polypeptide having 70%, 75%, 80%, 85%, 90%, 95%, 96%, 97%, 98%, 99%, 99.5% or 100% sequence identity to the polypeptide sequence of SEQ ID NO:131; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:134; a polypeptide having 70%, 75%, 80%, 85%, 90%, 95%, 96%, 97%, 98%, 99%, 99.5% or 100% sequence identity to the polypeptide sequence of SEQ ID NO: 128; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO:135; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 137; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:9; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO:142; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO: 129; and a polypeptide having 70%, 75%, 80%, 85%, 90%, 95%, 96%, 97%, 98%, 99%, 99.5% or 100% sequence identity to the polypeptide sequence of SEQ ID NO: 128. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO:142; a polypeptide having 70%, 75%, 80%, 85%, 90%, 95%, 96%, 97%, 98%, 99%, 99.5% or 100% sequence identity to the polypeptide sequence of SEQ ID NO: 144; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO:140; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 146; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO:133; and a polypeptide having 70%, 75%, 80%, 85%, 90%, 95%, 96%, 97%, 98%, 99%, 99.5% or 100% sequence identity to the polypeptide sequence of SEQ ID NO: 130. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO:128; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO:147; a polypeptide having 70%, 75%, 80%, 85%, 90%, 95%, 96%, 97%, 98%, 99%, 99.5% or 100% sequence identity to the polypeptide sequence of SEQ ID NO: 144; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:9; a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:147; a polypeptide having 70%, 75%, 80%, 85%, 90%, 95%, 96%, 97%, 98%, 99%, 99.5% or 100% sequence identity to the polypeptide sequence of SEQ ID NO: 142; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 129; a polypeptide having 70%, 75%, 80%, 85%, 90%, 95%, 96%, 97%, 98%, 99%, 99.5% or 100% sequence identity to the polypeptide sequence of SEQ ID NO: 128; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO:129; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 129; and a polypeptide having 70%, 75%, 80%, 85%, 90%, 95%, 96%, 97%, 98%, 99%, 99.5% or 100% sequence identity to the polypeptide sequence of SEQ ID NO:125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; and a polypeptide having 70%, 75%, 80%, 85%, 90%, 95%, 96%, 97%, 98%, 99%, 99.5% or 100% sequence identity to the polypeptide sequence of SEQ ID NO:127. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 127; and a polypeptide having 70%, 75%, 80%, 85%, 90%, 95%, 96%, 97%, 98%, 99%, 99.5% or 100% sequence identity to the polypeptide sequence of SEQ ID NO:125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 137; a polypeptide having 70%, 75%, 80%, 85%, 90%, 95%, 96%, 97%, 98%, 99%, 99.5% or 100% sequence identity to the polypeptide sequence of SEQ ID NO:144; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 129; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO:143; a polypeptide having 70%, 75%, 80%, 85%, 90%, 95%, 96%, 97%, 98%, 99%, 99.5% or 100% sequence identity to the polypeptide sequence of SEQ ID NO: 148; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; and a polypeptide having 70%, 75%, 80%, 85%, 90%, 95%, 96%, 97%, 98%, 99%, 99.5% or 100% sequence identity to the polypeptide sequence of SEQ ID NO: 130. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 127; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO:128; a polypeptide having 70%, 75%, 80%, 85%, 90%, 95%, 96%, 97%, 98%, 99%, 99.5% or 100% sequence identity to the polypeptide sequence of SEQ ID NO: 140; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 136. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 128. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:127; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO:124; a polypeptide having 70%, 75%, 80%, 85%, 90%, 95%, 96%, 97%, 98%, 99%, 99.5% or 100% sequence identity to the polypeptide sequence of SEQ ID NO: 122; and a polypeptide having 70%, 75%, 80%, 85%, 90%, 95%, 96%, 97%, 98%, 99%, 99.5% or 100% sequence identity to the polypeptide sequence of SEQ ID NO:131. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO:148; a polypeptide having 70%, 75%, 80%, 85%, 90%, 95%, 96%, 97%, 98%, 99%, 99.5% or 100% sequence identity to the polypeptide sequence of SEQ ID NO: 142; a polypeptide having 70%, 75%, 80%, 85%, 90%, 95%, 96%, 97%, 98%, 99%, 99.5% or 100% sequence identity to the polypeptide sequence of SEQ ID NO: 145; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO:145; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO:148; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 128; and a polypeptide having 70%, 75%, 80%, 85%, 90%, 95%, 96%, 97%, 98%, 99%, 99.5% or 100% sequence identity to the polypeptide sequence of SEQ ID NO:130. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO: 125; a polypeptide having 70%, 75%, 80%, 85%, 90%, 95%, 96%, 97%, 98%, 99%, 99.5% or 100% sequence identity to the polypeptide sequence of SEQ ID NO: 6; a polypeptide having 70%, 75%, 80%, 85%, 90%, 95%, 96%, 97%, 98%, 99%, 99.5% or 100% sequence identity to the polypeptide sequence of SEQ ID NO: 11; and a polypeptide having 70%, 75%, 80%, 85%, 90%, 95%, 96%, 97%, 98%, 99%, 99.5% or 100% sequence identity to the polypeptide sequence of SEQ ID NO: 7. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO: 6; a polypeptide having 70%, 75%, 80%, 85%, 90%, 95%, 96%, 97%, 98%, 99%, 99.5% or 100% sequence identity to the polypeptide sequence of SEQ ID NO: 123; and a polypeptide having 70%, 75%, 80%, 85%, 90%, 95%, 96%, 97%, 98%, 99%, 99.5% or 100% sequence identity to the polypeptide sequence of SEQ ID NO: 7. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 132; and a polypeptide having 70%, 75%, 80%, 85%, 90%, 95%, 96%, 97%, 98%, 99%, 99.5% or 100% sequence identity to the polypeptide sequence of SEQ ID NO:131. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:128; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO:125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:130; and a polypeptide having 70%, 75%, 80%, 85%, 90%, 95%, 96%, 97%, 98%, 99%, 99.5% or 100% sequence identity to the polypeptide sequence of SEQ ID NO: 137. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:141; a polypeptide having 70%, 75%, 80%, 85%, 90%, 95%, 96%, 97%, 98%, 99%, 99.5% or 100% sequence identity to the polypeptide sequence of SEQ ID NO:145; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 148; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO:127; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO:140; and a polypeptide having 70%, 75%, 80%, 85%, 90%, 95%, 96%, 97%, 98%, 99%, 99.5% or 100% sequence identity to the polypeptide sequence of SEQ ID NO: 143. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 127; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO:128; a polypeptide having 70%, 75%, 80%, 85%, 90%, 95%, 96%, 97%, 98%, 99%, 99.5% or 100% sequence identity to the polypeptide sequence of SEQ ID NO: 143; and a polypeptide having 70%, 75%, 80%, 85%, 90%, 95%, 96%, 97%, 98%, 99%, 99.5% or 100% sequence identity to the polypeptide sequence of SEQ ID NO: 148. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO:131. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 130. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO:145; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 137; a polypeptide having 70%, 75%, 80%, 85%, 90%, 95%, 96%, 97%, 98%, 99%, 99.5% or 100% sequence identity to the polypeptide sequence of SEQ ID NO:144; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO:143; a polypeptide having 70%, 75%, 80%, 85%, 90%, 95%, 96%, 97%, 98%, 99%, 99.5% or 100% sequence identity to the polypeptide sequence of SEQ ID NO: 137; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 11; and a polypeptide having 70%, 75%, 80%, 85%, 90%, 95%, 96%, 97%, 98%, 99%, 99.5% or 100% sequence identity to the polypeptide sequence of SEQ ID NO: 136. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:141; a polypeptide having 70%, 75%, 80%, 85%, 90%, 95%, 96%, 97%, 98%, 99%, 99.5% or 100% sequence identity to the polypeptide sequence of SEQ ID NO:145; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO: 143; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:124; and a polypeptide having 70%, 75%, 80%, 85%, 90%, 95%, 96%, 97%, 98%, 99%, 99.5% or 100% sequence identity to the polypeptide sequence of SEQ ID NO:131. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:124; a polypeptide having 70%, 75%, 80%, 85%, 90%, 95%, 96%, 97%, 98%, 99%, 99.5% or 100% sequence identity to the polypeptide sequence of SEQ ID NO: 127; and a polypeptide having 70%, 75%, 80%, 85%, 90%, 95%, 96%, 97%, 98%, 99%, 99.5% or 100% sequence identity to the polypeptide sequence of SEQ ID NO: 136. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 127; a polypeptide having 70%, 75%, 80%, 85%, 90%, 95%, 96%, 97%, 98%, 99%, 99.5% or 100% sequence identity to the polypeptide sequence of SEQ ID NO:145; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO: 143; and a polypeptide having 70%, 75%, 80%, 85%, 90%, 95%, 96%, 97%, 98%, 99%, 99.5% or 100% sequence identity to the polypeptide sequence of SEQ ID NO: 137. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:146; a polypeptide having 70%, 75%, 80%, 85%, 90%, 95%, 96%, 97%, 98%, 99%, 99.5% or 100% sequence identity to the polypeptide sequence of SEQ ID NO: 139; a polypeptide having 70%, 75%, 80%, 85%, 90%, 95%, 96%, 97%, 98%, 99%, 99.5% or 100% sequence identity to the polypeptide sequence of SEQ ID NO: 142; a polypeptide having 70%, 75%, 80%, 85%, 90%, 95%, 96%, 97%, 98%, 99%, 99.5% or 100% sequence identity to the polypeptide sequence of SEQ ID NO:145; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO: 11; and a polypeptide having 70%, 75%, 80%, 85%, 90%, 95%, 96%, 97%, 98%, 99%, 99.5% or 100% sequence identity to the polypeptide sequence of SEQ ID NO:131. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; and a polypeptide having 70%, 75%, 80%, 85%, 90%, 95%, 96%, 97%, 98%, 99%, 99.5% or 100% sequence identity to the polypeptide sequence of SEQ ID NO: 128. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:141; a polypeptide having 70%, 75%, 80%, 85%, 90%, 95%, 96%, 97%, 98%, 99%, 99.5% or 100% sequence identity to the polypeptide sequence of SEQ ID NO: 127; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO:140; a polypeptide having 70%, 75%, 80%, 85%, 90%, 95%, 96%, 97%, 98%, 99%, 99.5% or 100% sequence identity to the polypeptide sequence of SEQ ID NO: 137; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO: 140; and a polypeptide having 70%, 75%, 80%, 85%, 90%, 95%, 96%, 97%, 98%, 99%, 99.5% or 100% sequence identity to the polypeptide sequence of SEQ ID NO: 136. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:135; and a polypeptide having 70%, 75%, 80%, 85%, 90%, 95%, 96%, 97%, 98%, 99%, 99.5% or 100% sequence identity to the polypeptide sequence of SEQ ID NO: 126. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 127; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO:128; a polypeptide having 70%, 75%, 80%, 85%, 90%, 95%, 96%, 97%, 98%, 99%, 99.5% or 100% sequence identity to the polypeptide sequence of SEQ ID NO: 148; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO: 142; a polypeptide having 70%, 75%, 80%, 85%, 90%, 95%, 96%, 97%, 98%, 99%, 99.5% or 100% sequence identity to the polypeptide sequence of SEQ ID NO: 144; a polypeptide having 70%, 75%, 80%, 85%, 90%, 95%, 96%, 97%, 98%, 99%, 99.5% or 100% sequence identity to the polypeptide sequence of SEQ ID NO: 145; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:124; and a polypeptide having 70%, 75%, 80%, 85%, 90%, 95%, 96%, 97%, 98%, 99%, 99.5% or 100% sequence identity to the polypeptide sequence of SEQ ID NO: 12. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 149; a polypeptide having 70%, 75%, 80%, 85%, 90%, 95%, 96%, 97%, 98%, 99%, 99.5% or 100% sequence identity to the polypeptide sequence of SEQ ID NO:148; a polypeptide having 70%, 75%, 80%, 85%, 90%, 95%, 96%, 97%, 98%, 99%, 99.5% or 100% sequence identity to the polypeptide sequence of SEQ ID NO: 145; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:141; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO:143; a polypeptide having 70%, 75%, 80%, 85%, 90%, 95%, 96%, 97%, 98%, 99%, 99.5% or 100% sequence identity to the polypeptide sequence of SEQ ID NO: 146; and a polypeptide having 70%, 75%, 80%, 85%, 90%, 95%, 96%, 97%, 98%, 99%, 99.5% or 100% sequence identity to the polypeptide sequence of SEQ ID NO: 137. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO:137; a polypeptide having 70%, 75%, 80%, 85%, 90%, 95%, 96%, 97%, 98%, 99%, 99.5% or 100% sequence identity to the polypeptide sequence of SEQ ID NO: 144; a polypeptide having 70%, 75%, 80%, 85%, 90%, 95%, 96%, 97%, 98%, 99%, 99.5% or 100% sequence identity to the polypeptide sequence of SEQ ID NO: 145; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:147; a polypeptide having 70%, 75%, 80%, 85%, 90%, 95%, 96%, 97%, 98%, 99%, 99.5% or 100% sequence identity to the polypeptide sequence of SEQ ID NO: 137; a polypeptide having 70%, 75%, 80%, 85%, 90%, 95%, 96%, 97%, 98%, 99%, 99.5% or 100% sequence identity to the polypeptide sequence of SEQ ID NO: 142; a polypeptide having 70%, 75%, 80%, 85%, 90%, 95%, 96%, 97%, 98%, 99%, 99.5% or 100% sequence identity to the polypeptide sequence of SEQ ID NO: 145; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO: 127; and a polypeptide having 70%, 75%, 80%, 85%, 90%, 95%, 96%, 97%, 98%, 99%, 99.5% or 100% sequence identity to the polypeptide sequence of SEQ ID NO: 137. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 126. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:149; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO: 145; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; and a polypeptide having 70%, 75%, 80%, 85%, 90%, 95%, 96%, 97%, 98%, 99%, 99.5% or 100% sequence identity to the polypeptide sequence of SEQ ID NO: 130. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO:143; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:142; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:128; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO: 145; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 136. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 143; and a polypeptide having 70%, 75%, 80%, 85%, 90%, 95%, 96%, 97%, 98%, 99%, 99.5% or 100% sequence identity to the polypeptide sequence of SEQ ID NO:125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:125; and a polypeptide having 70%, 75%, 80%, 85%, 90%, 95%, 96%, 97%, 98%, 99%, 99.5% or 100% sequence identity to the polypeptide sequence of SEQ ID NO: 126. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 127; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO:128; a polypeptide having 70%, 75%, 80%, 85%, 90%, 95%, 96%, 97%, 98%, 99%, 99.5% or 100% sequence identity to the polypeptide sequence of SEQ ID NO: 143; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 149; a polypeptide having 70%, 75%, 80%, 85%, 90%, 95%, 96%, 97%, 98%, 99%, 99.5% or 100% sequence identity to the polypeptide sequence of SEQ ID NO: 148; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:147; a polypeptide having 70%, 75%, 80%, 85%, 90%, 95%, 96%, 97%, 98%, 99%, 99.5% or 100% sequence identity to the polypeptide sequence of SEQ ID NO: 137; a polypeptide having 70%, 75%, 80%, 85%, 90%, 95%, 96%, 97%, 98%, 99%, 99.5% or 100% sequence identity to the polypeptide sequence of SEQ ID NO: 144; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; and a polypeptide having 70%, 75%, 80%, 85%, 90%, 95%, 96%, 97%, 98%, 99%, 99.5% or 100% sequence identity to the polypeptide sequence of SEQ ID NO: 122. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO:11; a polypeptide having 70%, 75%, 80%, 85%, 90%, 95%, 96%, 97%, 98%, 99%, 99.5% or 100% sequence identity to the polypeptide sequence of SEQ ID NO:9; a polypeptide having 70%, 75%, 80%, 85%, 90%, 95%, 96%, 97%, 98%, 99%, 99.5% or 100% sequence identity to the polypeptide sequence of SEQ ID NO: 149; a polypeptide having 70%, 75%, 80%, 85%, 90%, 95%, 96%, 97%, 98%, 99%, 99.5% or 100% sequence identity to the polypeptide sequence of SEQ ID NO:148; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:124; a polypeptide having 70%, 75%, 80%, 85%, 90%, 95%, 96%, 97%, 98%, 99%, 99.5% or 100% sequence identity to the polypeptide sequence of SEQ ID NO: 133; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 125; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO:137; a polypeptide having 70%, 75%, 80%, 85%, 90%, 95%, 96%, 97%, 98%, 99%, 99.5% or 100% sequence identity to the polypeptide sequence of SEQ ID NO: 144; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO:143; a polypeptide having 70%, 75%, 80%, 85%, 90%, 95%, 96%, 97%, 98%, 99%, 99.5% or 100% sequence identity to the polypeptide sequence of SEQ ID NO: 149; a polypeptide having 70%, 75%, 80%, 85%, 90%, 95%, 96%, 97%, 98%, 99%, 99.5% or 100% sequence identity to the polypeptide sequence of SEQ ID NO: 148; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:127; and a polypeptide having 70%, 75%, 80%, 85%, 90%, 95%, 96%, 97%, 98%, 99%, 99.5% or 100% sequence identity to the polypeptide sequence of SEQ ID NO:131. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO:128; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO:148; a polypeptide having 70%, 75%, 80%, 85%, 90%, 95%, 96%, 97%, 98%, 99%, 99.5% or 100% sequence identity to the polypeptide sequence of SEQ ID NO: 137; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:9; a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:147; a polypeptide having 70%, 75%, 80%, 85%, 90%, 95%, 96%, 97%, 98%, 99%, 99.5% or 100% sequence identity to the polypeptide sequence of SEQ ID NO: 144; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO:140; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 148; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO:149; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; and a polypeptide having 70%, 75%, 80%, 85%, 90%, 95%, 96%, 97%, 98%, 99%, 99.5% or 100% sequence identity to the polypeptide sequence of SEQ ID NO: 132. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 11; a polypeptide having 70%, 75%, 80%, 85%, 90%, 95%, 96%, 97%, 98%, 99%, 99.5% or 100% sequence identity to the polypeptide sequence of SEQ ID NO:9; a polypeptide having 70%, 75%, 80%, 85%, 90%, 95%, 96%, 97%, 98%, 99%, 99.5% or 100% sequence identity to the polypeptide sequence of SEQ ID NO:126; a polypeptide having 70%, 75%, 80%, 85%, 90%, 95%, 96%, 97%, 98%, 99%, 99.5% or 100% sequence identity to the polypeptide sequence of SEQ ID NO: 148; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 135; a polypeptide having 70%, 75%, 80%, 85%, 90%, 95%, 96%, 97%, 98%, 99%, 99.5% or 100% sequence identity to the polypeptide sequence of SEQ ID NO: 128; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; and a polypeptide having 70%, 75%, 80%, 85%, 90%, 95%, 96%, 97%, 98%, 99%, 99.5% or 100% sequence identity to the polypeptide sequence of SEQ ID NO: 135. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO:145; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO:147; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO:137; a polypeptide having 70%, 75%, 80%, 85%, 90%, 95%, 96%, 97%, 98%, 99%, 99.5% or 100% sequence identity to the polypeptide sequence of SEQ ID NO: 144; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:144; a polypeptide having 70%, 75%, 80%, 85%, 90%, 95%, 96%, 97%, 98%, 99%, 99.5% or 100% sequence identity to the polypeptide sequence of SEQ ID NO: 145; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO:6; and a polypeptide having 70%, 75%, 80%, 85%, 90%, 95%, 96%, 97%, 98%, 99%, 99.5% or 100% sequence identity to the polypeptide sequence of SEQ ID NO: 134. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO:145; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO:137; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO: 128; and a polypeptide having 70%, 75%, 80%, 85%, 90%, 95%, 96%, 97%, 98%, 99%, 99.5% or 100% sequence identity to the polypeptide sequence of SEQ ID NO: 126. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 11. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:141; a polypeptide having 70%, 75%, 80%, 85%, 90%, 95%, 96%, 97%, 98%, 99%, 99.5% or 100% sequence identity to the polypeptide sequence of SEQ ID NO:127; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 147; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO:141; and a polypeptide having 70%, 75%, 80%, 85%, 90%, 95%, 96%, 97%, 98%, 99%, 99.5% or 100% sequence identity to the polypeptide sequence of SEQ ID NO: 130. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; and a polypeptide having 70%, 75%, 80%, 85%, 90%, 95%, 96%, 97%, 98%, 99%, 99.5% or 100% sequence identity to the polypeptide sequence of SEQ ID NO: 124. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO:142; a polypeptide having 70%, 75%, 80%, 85%, 90%, 95%, 96%, 97%, 98%, 99%, 99.5% or 100% sequence identity to the polypeptide sequence of SEQ ID NO: 145; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO:137; a polypeptide having 70%, 75%, 80%, 85%, 90%, 95%, 96%, 97%, 98%, 99%, 99.5% or 100% sequence identity to the polypeptide sequence of SEQ ID NO: 142; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 129; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO: 143; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO:128; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 132. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 126. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:134; and a polypeptide having 70%, 75%, 80%, 85%, 90%, 95%, 96%, 97%, 98%, 99%, 99.5% or 100% sequence identity to the polypeptide sequence of SEQ ID NO:125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:147; a polypeptide having 70%, 75%, 80%, 85%, 90%, 95%, 96%, 97%, 98%, 99%, 99.5% or 100% sequence identity to the polypeptide sequence of SEQ ID NO: 137; a polypeptide having 70%, 75%, 80%, 85%, 90%, 95%, 96%, 97%, 98%, 99%, 99.5% or 100% sequence identity to the polypeptide sequence of SEQ ID NO: 142; a polypeptide having 70%, 75%, 80%, 85%, 90%, 95%, 96%, 97%, 98%, 99%, 99.5% or 100% sequence identity to the polypeptide sequence of SEQ ID NO:144; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO:143; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO:138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:141; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO:143; a polypeptide having 70%, 75%, 80%, 85%, 90%, 95%, 96%, 97%, 98%, 99%, 99.5% or 100% sequence identity to the polypeptide sequence of SEQ ID NO: 145; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 129; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:127; and a polypeptide having 70%, 75%, 80%, 85%, 90%, 95%, 96%, 97%, 98%, 99%, 99.5% or 100% sequence identity to the polypeptide sequence of SEQ ID NO: 126. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO: 134; and a polypeptide having 70%, 75%, 80%, 85%, 90%, 95%, 96%, 97%, 98%, 99%, 99.5% or 100% sequence identity to the polypeptide sequence of SEQ ID NO: 132. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 132; and a polypeptide having 70%, 75%, 80%, 85%, 90%, 95%, 96%, 97%, 98%, 99%, 99.5% or 100% sequence identity to the polypeptide sequence of SEQ ID NO:138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO:133; a polypeptide having 70%, 75%, 80%, 85%, 90%, 95%, 96%, 97%, 98%, 99%, 99.5% or 100% sequence identity to the polypeptide sequence of SEQ ID NO:125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO: 11; a polypeptide having 70%, 75%, 80%, 85%, 90%, 95%, 96%, 97%, 98%, 99%, 99.5% or 100% sequence identity to the polypeptide sequence of SEQ ID NO:9; a polypeptide having 70%, 75%, 80%, 85%, 90%, 95%, 96%, 97%, 98%, 99%, 99.5% or 100% sequence identity to the polypeptide sequence of SEQ ID NO:148; a polypeptide having 70%, 75%, 80%, 85%, 90%, 95%, 96%, 97%, 98%, 99%, 99.5% or 100% sequence identity to the polypeptide sequence of SEQ ID NO: 142; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:141; a polypeptide having 70%, 75%, 80%, 85%, 90%, 95%, 96%, 97%, 98%, 99%, 99.5% or 100% sequence identity to the polypeptide sequence of SEQ ID NO: 127; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO:143; a polypeptide having 70%, 75%, 80%, 85%, 90%, 95%, 96%, 97%, 98%, 99%, 99.5% or 100% sequence identity to the polypeptide sequence of SEQ ID NO: 137; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO:140; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 148; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:147; a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 142; a polypeptide having 70%, 75%, 80%, 85%, 90%, 95%, 96%, 97%, 98%, 99%, 99.5% or 100% sequence identity to the polypeptide sequence of SEQ ID NO:145; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO:143; a polypeptide having 70%, 75%, 80%, 85%, 90%, 95%, 96%, 97%, 98%, 99%, 99.5% or 100% sequence identity to the polypeptide sequence of SEQ ID NO: 146; and a polypeptide having 70%, 75%, 80%, 85%, 90%, 95%, 96%, 97%, 98%, 99%, 99.5% or 100% sequence identity to the polypeptide sequence of SEQ ID NO: 137. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 142. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO:128; and a polypeptide having 70%, 75%, 80%, 85%, 90%, 95%, 96%, 97%, 98%, 99%, 99.5% or 100% sequence identity to the polypeptide sequence of SEQ ID NO:131. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO:145; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO:143; and a polypeptide having 70%, 75%, 80%, 85%, 90%, 95%, 96%, 97%, 98%, 99%, 99.5% or 100% sequence identity to the polypeptide sequence of SEQ ID NO: 148. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:124; a polypeptide having 70%, 75%, 80%, 85%, 90%, 95%, 96%, 97%, 98%, 99%, 99.5% or 100% sequence identity to the polypeptide sequence of SEQ ID NO:133; a polypeptide having 70%, 75%, 80%, 85%, 90%, 95%, 96%, 97%, 98%, 99%, 99.5% or 100% sequence identity to the polypeptide sequence of SEQ ID NO:131; and a polypeptide having 70%, 75%, 80%, 85%, 90%, 95%, 96%, 97%, 98%, 99%, 99.5% or 100% sequence identity to the polypeptide sequence of SEQ ID NO: 139. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO:149; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO: 137; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO:145; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO:137; and a polypeptide having 70%, 75%, 80%, 85%, 90%, 95%, 96%, 97%, 98%, 99%, 99.5% or 100% sequence identity to the polypeptide sequence of SEQ ID NO: 144. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:143; a polypeptide having 70%, 75%, 80%, 85%, 90%, 95%, 96%, 97%, 98%, 99%, 99.5% or 100% sequence identity to the polypeptide sequence of SEQ ID NO:147; a polypeptide having 70%, 75%, 80%, 85%, 90%, 95%, 96%, 97%, 98%, 99%, 99.5% or 100% sequence identity to the polypeptide sequence of SEQ ID NO:142; a polypeptide having 70%, 75%, 80%, 85%, 90%, 95%, 96%, 97%, 98%, 99%, 99.5% or 100% sequence identity to the polypeptide sequence of SEQ ID NO: 145; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 134. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO:147; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 137; a polypeptide having 70%, 75%, 80%, 85%, 90%, 95%, 96%, 97%, 98%, 99%, 99.5% or 100% sequence identity to the polypeptide sequence of SEQ ID NO:142; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 127; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO:140; a polypeptide having 70%, 75%, 80%, 85%, 90%, 95%, 96%, 97%, 98%, 99%, 99.5% or 100% sequence identity to the polypeptide sequence of SEQ ID NO: 143; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 12. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:141; a polypeptide having 70%, 75%, 80%, 85%, 90%, 95%, 96%, 97%, 98%, 99%, 99.5% or 100% sequence identity to the polypeptide sequence of SEQ ID NO:145; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 137; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:130; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:147; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO:142; a polypeptide having 70%, 75%, 80%, 85%, 90%, 95%, 96%, 97%, 98%, 99%, 99.5% or 100% sequence identity to the polypeptide sequence of SEQ ID NO: 145; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 129; and a polypeptide having 70%, 75%, 80%, 85%, 90%, 95%, 96%, 97%, 98%, 99%, 99.5% or 100% sequence identity to the polypeptide sequence of SEQ ID NO: 132. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 149; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 127; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO:143; a polypeptide having 70%, 75%, 80%, 85%, 90%, 95%, 96%, 97%, 98%, 99%, 99.5% or 100% sequence identity to the polypeptide sequence of SEQ ID NO: 137; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 127; and a polypeptide having 70%, 75%, 80%, 85%, 90%, 95%, 96%, 97%, 98%, 99%, 99.5% or 100% sequence identity to the polypeptide sequence of SEQ ID NO: 12. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO:135; and a polypeptide having 70%, 75%, 80%, 85%, 90%, 95%, 96%, 97%, 98%, 99%, 99.5% or 100% sequence identity to the polypeptide sequence of SEQ ID NO:131. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO:128; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO:142; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO:147; a polypeptide having 70%, 75%, 80%, 85%, 90%, 95%, 96%, 97%, 98%, 99%, 99.5% or 100% sequence identity to the polypeptide sequence of SEQ ID NO: 142; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO:138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO:124; and a polypeptide having 70%, 75%, 80%, 85%, 90%, 95%, 96%, 97%, 98%, 99%, 99.5% or 100% sequence identity to the polypeptide sequence of SEQ ID NO:130. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO:128; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO:144; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:123; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 125. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO: 135; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:143; a polypeptide having 70%, 75%, 80%, 85%, 90%, 95%, 96%, 97%, 98%, 99%, 99.5% or 100% sequence identity to the polypeptide sequence of SEQ ID NO: 148; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO:128; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO: 143; and a polypeptide having 70%, 75%, 80%, 85%, 90%, 95%, 96%, 97%, 98%, 99%, 99.5% or 100% sequence identity to the polypeptide sequence of SEQ ID NO: 146. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 129. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:124; a polypeptide having 70%, 75%, 80%, 85%, 90%, 95%, 96%, 97%, 98%, 99%, 99.5% or 100% sequence identity to the polypeptide sequence of SEQ ID NO: 133; and a polypeptide having 70%, 75%, 80%, 85%, 90%, 95%, 96%, 97%, 98%, 99%, 99.5% or 100% sequence identity to the polypeptide sequence of SEQ ID NO: 139. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO:147; a polypeptide having 70%, 75%, 80%, 85%, 90%, 95%, 96%, 97%, 98%, 99%, 99.5% or 100% sequence identity to the polypeptide sequence of SEQ ID NO: 142; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 127; a polypeptide having 70%, 75%, 80%, 85%, 90%, 95%, 96%, 97%, 98%, 99%, 99.5% or 100% sequence identity to the polypeptide sequence of SEQ ID NO: 145; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 148; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO:128; a polypeptide having 70%, 75%, 80%, 85%, 90%, 95%, 96%, 97%, 98%, 99%, 99.5% or 100% sequence identity to the polypeptide sequence of SEQ ID NO: 140; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO:137; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO:146; a polypeptide having 70%, 75%, 80%, 85%, 90%, 95%, 96%, 97%, 98%, 99%, 99.5% or 100% sequence identity to the polypeptide sequence of SEQ ID NO: 148; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:141; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO:149; a polypeptide having 70%, 75%, 80%, 85%, 90%, 95%, 96%, 97%, 98%, 99%, 99.5% or 100% sequence identity to the polypeptide sequence of SEQ ID NO: 148; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO:140; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 144; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:124; and a polypeptide having 70%, 75%, 80%, 85%, 90%, 95%, 96%, 97%, 98%, 99%, 99.5% or 100% sequence identity to the polypeptide sequence of SEQ ID NO: 129. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO: 147; a polypeptide having 70%, 75%, 80%, 85%, 90%, 95%, 96%, 97%, 98%, 99%, 99.5% or 100% sequence identity to the polypeptide sequence of SEQ ID NO:142; a polypeptide having 70%, 75%, 80%, 85%, 90%, 95%, 96%, 97%, 98%, 99%, 99.5% or 100% sequence identity to the polypeptide sequence of SEQ ID NO: 144; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 124; and a polypeptide having 70%, 75%, 80%, 85%, 90%, 95%, 96%, 97%, 98%, 99%, 99.5% or 100% sequence identity to the polypeptide sequence of SEQ ID NO: 134. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 141; a polypeptide having 70%, 75%, 80%, 85%, 90%, 95%, 96%, 97%, 98%, 99%, 99.5% or 100% sequence identity to the polypeptide sequence of SEQ ID NO: 128; a polypeptide having 70%, 75%, 80%, 85%, 90%, 95%, 96%, 97%, 98%, 99%, 99.5% or 100% sequence identity to the polypeptide sequence of SEQ ID NO:143; a polypeptide having 70%, 75%, 80%, 85%, 90%, 95%, 96%, 97%, 98%, 99%, 99.5% or 100% sequence identity to the polypeptide sequence of SEQ ID NO: 137; a polypeptide having 70%, 75%, 80%, 85%, 90%, 95%, 96%, 97%, 98%, 99%, 99.5% or 100% sequence identity to the polypeptide sequence of SEQ ID NO: 144; a polypeptide having 70%, 75%, 80%, 85%, 90%, 95%, 96%, 97%, 98%, 99%, 99.5% or 100% sequence identity to the polypeptide sequence of SEQ ID NO:138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:124; and a polypeptide having 70%, 75%, 80%, 85%, 90%, 95%, 96%, 97%, 98%, 99%, 99.5% or 100% sequence identity to the polypeptide sequence of SEQ ID NO:131. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO:147; a polypeptide having 70%, 75%, 80%, 85%, 90%, 95%, 96%, 97%, 98%, 99%, 99.5% or 100% sequence identity to the polypeptide sequence of SEQ ID NO: 142; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 132; and a polypeptide having 70%, 75%, 80%, 85%, 90%, 95%, 96%, 97%, 98%, 99%, 99.5% or 100% sequence identity to the polypeptide sequence of SEQ ID NO: 12. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO:124; a polypeptide having 70%, 75%, 80%, 85%, 90%, 95%, 96%, 97%, 98%, 99%, 99.5% or 100% sequence identity to the polypeptide sequence of SEQ ID NO:129; and a polypeptide having 70%, 75%, 80%, 85%, 90%, 95%, 96%, 97%, 98%, 99%, 99.5% or 100% sequence identity to the polypeptide sequence of SEQ ID NO:131. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 121; a polypeptide having 70%, 75%, 80%, 85%, 90%, 95%, 96%, 97%, 98%, 99%, 99.5% or 100% sequence identity to the polypeptide sequence of SEQ ID NO:122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:124; and a polypeptide having 70%, 75%, 80%, 85%, 90%, 95%, 96%, 97%, 98%, 99%, 99.5% or 100% sequence identity to the polypeptide sequence of SEQ ID NO: 12. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 124; a polypeptide having 70%, 75%, 80%, 85%, 90%, 95%, 96%, 97%, 98%, 99%, 99.5% or 100% sequence identity to the polypeptide sequence of SEQ ID NO: 143; a polypeptide having 70%, 75%, 80%, 85%, 90%, 95%, 96%, 97%, 98%, 99%, 99.5% or 100% sequence identity to the polypeptide sequence of SEQ ID NO: 149; a polypeptide having 70%, 75%, 80%, 85%, 90%, 95%, 96%, 97%, 98%, 99%, 99.5% or 100% sequence identity to the polypeptide sequence of SEQ ID NO:148; a polypeptide having 70%, 75%, 80%, 85%, 90%, 95%, 96%, 97%, 98%, 99%, 99.5% or 100% sequence identity to the polypeptide sequence of SEQ ID NO: 147; and a polypeptide having 70%, 75%, 80%, 85%, 90%, 95%, 96%, 97%, 98%, 99%, 99.5% or 100% sequence identity to the polypeptide sequence of SEQ ID NO: 138. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO:121; a polypeptide having 70%, 75%, 80%, 85%, 90%, 95%, 96%, 97%, 98%, 99%, 99.5% or 100% sequence identity to the polypeptide sequence of SEQ ID NO: 122; a polypeptide having 70%, 75%, 80%, 85%, 90%, 95%, 96%, 97%, 98%, 99%, 99.5% or 100% sequence identity to the polypeptide sequence of SEQ ID NO:7; a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO: 125; and a polypeptide having 70%, 75%, 80%, 85%, 90%, 95%, 96%, 97%, 98%, 99%, 99.5% or 100% sequence identity to the polypeptide sequence of SEQ ID NO:131. In some embodiments, the one or more isolated and purified tumor-associated proteins or fragments thereof can comprise: a polypeptide having 70%, 75%, 80%, 85%, 90%, 95%, 96%, 97%, 98%, 99%, 99.5% or 100% sequence identity to the polypeptide sequence of SEQ ID NO: 123; a polypeptide having 70%, 75%, 80%, 85%, 90%, 95%, 96%, 97%, 98%, 99%, 99.5% or 100% sequence identity to the polypeptide sequence of SEQ ID NO:9; a polypeptide having 70%, 75%, 80%, 85%, 90%, 95%, 96%, 97%, 98%, 99%, 99.5% or 100% sequence identity to the polypeptide sequence of SEQ ID NO: 126; a polypeptide having 70%, 75%, 80%, 85%, 90%, 95%, 96%, 97%, 98%, 99%, 99.5% or 100% sequence identity to the polypeptide sequence of SEQ ID NO: 148; a polypeptide having 70%, 75%, 80%, 85%, 90%, 95%, 96%, 97%, 98%, 99%, 99.5% or 100% sequence identity to the polypeptide sequence of SEQ ID NO:142; and a polypeptide having 70%, 75%, 80%, 85%, 90%, 95%, 96%, 97%, 98%, 99%, 99.5% or 100% sequence identity to the polypeptide sequence of SEQ ID NO:138.

In some embodiments, the panel of tumor-associated antigens may comprise both an IgA autoantigen and an IgG autoantigen. In some embodiments, the panel of tumor-associated proteins can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more IgA autoantigens. In some embodiments, the panel of tumor-associated proteins can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more IgG autoantigens. In some embodiments, the panel of tumor-associated proteins can comprise 1 IgA autoantigen and 5 IgG autoantigens. In some embodiments, the panel of tumor-associated proteins can comprise 2 IgA autoantigen and 4 IgG autoantigens. In some embodiments, the panel of tumor-associated proteins can comprise 3 IgA autoantigen and 3 IgG autoantigens. In some embodiments, the panel of tumor-associated proteins can comprise 4 IgA autoantigen and 2 IgG autoantigens. In some embodiments, the panel of tumor-associated proteins can comprise 5 IgA autoantigen and 1 IgG autoantigens. In some embodiments, the panel of tumor-associated proteins can comprise IgA autoantigens TRIM33, BCL7A, and MTERF4, and IgG autoantigens DDX4, CTAG1A, and MAGEC2. In some embodiments, the panel of tumor-associated proteins can comprise IgA autoantigens BCL7A, MTERF4, NUDT14, and IgG autoantigens DDX4, CTAG1A, and MAGEC2. In some embodiments, the panel of tumor-associated proteins can comprise IgA autoantigens TRIM33, BCL7A, and MTERF4, and IgG autoantigens CTAG1A, MAGEC2, and C1QTNF1. In some embodiments, the panel of tumor-associated proteins can comprise IgA autoantigens BCL7A, DDX4, and RALGDS, and IgG autoantigens DDX4, CTAG1A, and MAGEC2. In some embodiments, the panel of tumor-associated proteins can comprise IgA autoantigens TRIM33, BCL7A, and MTERF4, and IgG autoantigens DDX4, CTAG1A, MTERF4, and C1QTNF1. In some embodiments, the panel of tumor-associated proteins can comprise IgA autoantigens BCL7A, MTERF4, and APH1A, and IgG autoantigens DDX4, CTAG1A, MAGEC2. In some embodiments, the panel of tumor-associated proteins can comprise IgA autoantigens BCL7A, MTERF4, and N4BP1, and IgG autoantigens DDX4, CTAG1A, and MAGEC2. In some embodiments, the panel of tumor-associated proteins can comprise IgA autoantigens BCL7A, MTERF4, and SSBP4, and IgG autoantigens DDX4, CTAG1A, and MAGEC2. In some embodiments, the panel of tumor-associated proteins can comprise IgA autoantigens TRIM33, BCL7A, and APH1A, and IgG autoantigens DDX4, CTAG1A, and C1QTNF1. In some embodiments, the panel of tumor-associated proteins can comprise IgA autoantigens TRIM33, BCL7A, and KCNRG, and IgG autoantigens DDX4, CTAG1A, and C1QTNF1. In some embodiments, the panel of tumor-associated proteins can comprise IgA autoantigens TRIM33, BCL7A, and SSBP4-A, and IgG autoantigens DDX4, CTAG1A, and C1QTNF1.

In some embodiments, the panel of tumor-associated proteins can comprise IgA autoantigens TRIM33 and SSBP4, and IgG autoantigens TP53, NSG1, and CTAG1A. In some embodiments, the panel of tumor-associated proteins can comprise one, two, three, four or more of IgA autoantigens TRIM33 and SSBP4, and IgG autoantigens TP53, NSG1, and CTAG1A. In some embodiments, the panel of tumor-associated proteins can comprise IgA autoantigens TRIM33 and MTERF4, and IgG autoantigens TP53, MAGEC2, and CTAG1A. In some embodiments, the panel of tumor-associated proteins can comprise one, two, three, four or more of IgA autoantigens TRIM33 and MTERF4, and IgG autoantigens TP53, MAGEC2, and CTAG1A.

Coupling of Tumor-Associated Proteins to a Solid Support

A tumor-associated protein can be coupled to a solid support (e.g., an array or bead). In some instances, a tumor-associated protein is non-covalently coupled to a solid support. For example, a non-covalent interaction can be an ionic interaction or a van der Waals interaction. In some instances, a tumor-associated protein can be covalently coupled to a solid support. In some instances, a tumor-associated protein can be reversibly coupled to a solid support. In some instances, a tumor-associated protein is irreversibly coupled to a solid support.

A surface of a solid support can be coated with a functional group and a tumor-associated protein can be attached to the solid support through the functional group. For example, a solid support can be coated with a first functional group and a tumor-associated protein comprising a second functional group can be attached to the solid support by reacting the first functional group with the second functional group. For example, a surface of a solid support can be coated with streptavidin and a biotinylated tumor-associated protein can be attached thereto. Exemplary couplings of a tumor-associated protein include streptavidin- or avidin- to biotin interactions; hydrophobic interactions; magnetic interactions; polar interactions, (e.g., associations between two polar surfaces); formation of a covalent bond (e.g., an amide bond, disulfide bond, thioether bond, or via crosslinking agents; and via an acid-labile linker.

In some embodiments, the surface of a solid support can be coated with an affinity ligand. In some embodiments, an affinity ligand can include, but is not limited to an antigen, an antibody, an antibody fragment, glutathione, calmodulin, biotin, streptavidin, streptactin, amylose, an anion-exchange resin such as Mono-Q, FlAsH and ReAsH biarsenical compounds, pilin-C protein, SpyCatcher protein or a metal chelate. In some instances, the metal chelate can include but is not limited to nickel, cobalt, zinc, mercury, cupper or iron chelate. In some embodiments, the solid support can be coated entirely. In some embodiments, the solid support can be coated partially. In some embodiments, tumor-associated proteins can comprise an affinity tag and solid support can comprise an affinity ligand, thus coupling the tumor-associated proteins to the solid support by reacting the affinity tag with the affinity ligand.

In some instances, a tumor-associated protein is coupled to a solid surface through a linker. For example, a first functional group of a linker attached to a solid surface can be coupled to a tumor-associated protein, thereby coupling the tumor-associated protein to the solid surface. For example, a first functional group of a linker can be coupled to a tumor-associated protein and a second functional group of the linker can be coupled to a solid support, thereby coupling the tumor-associated protein to the solid surface. Tumor-associated proteins can be coupled to a solid surface through a linker. In some instances, a linker comprising a first and a second functional group can be attached to the solid support via the second functional group after the first functional group is coupled to the tumor-associated protein. In some instances, a linker comprising a first and a second functional group can be attached to the solid support via the second functional group before the first functional group is coupled to the tumor-associated protein.

In some instances, a tumor-associated protein is coupled to a solid surface via an antibody. For example, an antibody linker can be attached to a solid surface and a tumor-associated protein to which the antibody specifically bind can be linked to the solid support by binding to the antibody linker. In some instances, the coupling is photocleavable. In some instances, tumor-associated proteins can comprise a tag that is directly coupled to a solid surface. For example, a tumor-associated protein can comprise a fusion tag that is directly conjugated to the solid surface. For example, a tumor-associated protein can comprise a GST-tag, His-tag, FLAG-tag, or other similar tags and the tag can be directly coupled to the solid surface instead of the tumor-associated protein itself.

There are many known methods for covalently immobilizing polypeptides and antibodies onto a solid support. For example, MacBeath et al., (1999) J Am Chem Soc 121: 7967-68) use the Michael addition to link thiol-containing compounds to maleimide-derivatized glass slides to form a microarray of small molecules. (See also, Lam & Renil (2002) Current Opin Chemical Biol 6:353-58). Non-covalent coupling may be by any suitable secondary interaction, including but not limited to hydrophobic bonding, hydrogen bonding, Van der Waals interactions, ionic bonding, etc.

Amine chemistry can be used to couple or immobilize tumor-associated proteins to a solid surface. For example, a covalent amide bond can be formed between a tumor-associated protein and a solid support. For example, a covalent amide bond can be formed by reacting a carboxyl-functionalized tumor-associated protein with an amino-functionalized solid support. For example, a covalent amide bond can be formed by reacting an amide-functionalized tumor-associated protein with a carboxyl-functionalized solid support. Amine-terminated tumor-associated protein may be immobilized using amine/cyanuric chloride coupling; amide bonding through reactions with N-hydroxysuccinimide (NHS)-ester-, carboxylic acid-, carbonate-, anhydride- or acyl group-functionalized surfaces; amidine formation through reaction with imidoester-functionalized surfaces; sulphonamide formation through reactions with sulfonyl halide-functionalized surfaces; aniline formation through reactions with surface presenting aryl groups; imine formation through reactions with aldehyde-functionalized surfaces; amino ketone formation through Mannich reactions with aldehyde-functionalized surfaces; guanidine formation through reactions with carbodiimide-functionalized surfaces; urea formation through reactions with isocyanate-functionalized surfaces; thiourea formation through reactions with isothiocyanate-functionalized surfaces, or; amino alcohol formation through reactions with epoxide-functionalized surfaces. Hydrazine- or oxyamine-terminated binding agents may be immobilized in the same way.

Thiol groups can be used to couple or immobilize tumor-associated protein to a solid surface. For example, tumor-associated protein having or functionalized with thiol groups with may be immobilized on surfaces presenting, e.g., maleimide, aryl- or carbon-carbon double-bond-containing groups through formation of stable carbon-sulfur bonds, or through interactions with aziridine-functionalized surfaces. Disulfide exchange reactions with thiol-functionalized surfaces may also be used. tumor-associated protein having or functionalized with thiol groups may be immobilized on gold surfaces through semi-covalent interactions between gold and sulphur groups.

Carboxylic acid-functionalized surfaces may also be used to immobilize tumor-associated protein functionalized with carbodiimide and diazoalkane groups. Solid surfaces presenting hydroxyl groups may be used to immobilize isocyanate- and epoxide-functionalized tumor-associated proteins.

Functionalized tumor-associated protein may also be immobilized through cycloaddition reactions between functional groups having a conjugated diene and groups having a substituted alkene through Diels-Alder chemistry, or using “click” chemistry, through reactions between nitrile and azine groups. In any of the above described covalent couplings, the tumor-associated protein-surface orientation of functional groups may be reversed. An alternative means of covalent attachment not utilizing a derivatized binding agent utilizes array surfaces having photoreactive groups such as benzophenone, diazo, diazirine, phthalamido and arylazide groups.

Non-covalent immobilization may involve electrostatic interactions between tumor-associated proteins and surfaces modified to contain positively- or negatively-charged groups, such as amine or carboxy groups, respectively. tumor-associated proteins may be non-covalently immobilized in a defined orientation, for example, using fluorophilic, biotin-streptavidin, histidine-Ni, histidine-Co, and complementary single-stranded DNA interactions between tagged tumor-associated proteins and binding partner-coated surfaces, in either orientation.

Appropriate agents for coupling of tumor-associated proteins to a solid surface include a variety of agents that are capable of reacting with a functional group present on a surface of the tumor-associated protein and with a functional group present on the solid surface. Reagents capable of such reactivity include homo- and hetero-bifunctional reagents, many of which are known in the art. Exemplary bifunctional cross-linking agents include is N-succinimidyl(4-iodoacetyl) aminobenzoate (SIAB), dimaleimide, dithio-bis-nitrobenzoic acid (DTNB), N-succinimidyl-S-acetyl-thioacetate (SATA), N-succinimidyl-3-(2-pyridyldithio) propionate (SPDP), succinimidyl 4-(N-maleimidomethyl) cyclohexane-1-carboxylate (SMCC) and 6-hydrazinonicotimide (HYNIC). Any suitable nucleophile reactive group can be used including —NR1-NH2 (hydrazide), —NR1(C—O)NR2NH2 (semicarbazide), —NR1(C—S)NR2NH2 (thiosemicarbazide), (C—O)NR1NH2 (carbonylhydrazide), —(C—S)NR1NH2 (thiocarbonylhydrazide), —(SO2)NR1NH2 (sulfonylhydrazide), —NR1NR2(C)NR3NH2 (carbazide), —NR1NR2(C—S)NR3NH2 (thiocarbazide), and —O—NH2 (hydroxylamine), where each R1, R2, and R3 is independently H, or alkyl having 1-6 carbons. The nucleophilic moiety can include any suitable nucleophile, e.g., hydrazide, hydroxylamine, semicarbazide, or carbonylhydrazide.

In addition to those described above, other covalent and non-covalent means of attachment may be employed and are well known to those skilled in the art. A tumor-associated protein may be deposited onto a substrate or support by any suitable technique. For example, a tumor-associated protein may be deposited as a monolayer (e.g., a self-assembled monolayer), a continuous layer or as a discontinuous (e.g., patterned) layer. A tumor-associated protein may be deposited or coupled to a support or substrate by modification of the substrate or support by chemical reaction (See, e.g., U.S. Pat. No. 6,444,254), reactive plasma etching, corona discharge treatment, a plasma deposition process, spin coating, dip coating, spray painting, deposition, printing, stamping, diffusion, adsorption/absorption, covalent cross-linking, or combinations thereof. The tumor-associated proteins may be directly spotted onto a surface (e.g., a planar glass surface). In some instances, when necessary or beneficial to keep tumor-associated proteins (e.g., Abs) in a wet environment during the printing process, glycerol (30-40%) may be employed, and/or spotting can be carried out in a humidity-controlled environment.

Antibodies and Autoantibodies

An autoantibody can be an antibody produced by a subject's immune system that is directed against one or more of the subject's own molecules. An autoantibody should be understood to be an antibody, and an antibody may be an autoantibody. In some embodiments, an autoantibody is directed to one or more of a subject's own proteins. In some embodiments, an autoantibody binds to one or more of a subject's proteins. In some embodiments, an autoantibody binds to a tumor-associated protein. An antibody, including an autoantibody, can specifically bind to a particular portion of a protein, including a tumor-associated protein. The portion of a protein to which is an antibody binds may be called an antigenic sequence. The portion of a protein to which is an antibody binds may be also be referred to as an epitope or an antigenic determinant. An antigenic sequence or epitope may comprise a continuous sequence of amino acids, or it may comprise discontinuous sections of the target protein's amino acid sequence. In some embodiments, an antigenic sequence or epitope of a protein may comprise 1, 2, 3, 4, 5, 6, 7, 8, 9, 10, 11, 12, 13, 14, 15, 16, 17, 18, 19, or 20 amino acids. An antibody can have strong binding affinity for an antigenic sequence or epitope, as measured by its dissociation constant. In some embodiments, an antibody can have dissociation constant of less than about 10⁻⁵M, 10⁻⁶M, 10⁻⁷M, 10⁻⁸M, 10⁻⁹M, 10⁻¹⁰M, 10⁻¹¹M, 10⁻¹²M, 10⁻¹³M, or 10⁻¹⁴M. In some embodiments, an autoantibody can bind to an antigenic sequence having at least about 70%, 75%, 80%, 85%, 90%, 95%, 96%, 97%, 98%, 99%, or 99.5% sequence identity to any one of SEQ ID NOs: 1-21, or a fragment thereof. In some embodiments, an autoantibody can bind to an antigenic sequence as shown in any one of SEQ ID NOs: 22-120 or a fragment thereof. In some embodiments, an autoantibody can bind to an antigenic sequence having at least about 70%, 75%, 80%, 85%, 90%, 95%, 96%, 97%, 98%, 99%, or 99.5% sequence identity to any one of SEQ ID NOs: 22-120, or a fragment thereof.

An antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. A naturally occurring antibody can be a protein comprising at least two heavy (H) chains and two light (L) chains inter-connected by disulfide bonds. Each heavy chain can be comprised of a heavy chain variable region (V_{H}) and a heavy chain constant region. The heavy chain constant region can be comprised of three domains, C_{H1}, C_{H2} and C_{H3}. Each light chain can be comprised of a light chain variable region (V_{L}) and a light chain constant region. The light chain constant region can be comprised of one domain, C_{L}. The V_{H} and V_{L} regions can be further subdivided into regions of hypervariability, termed complementary determining regions (CDR), interspersed with regions that are more conserved, termed framework regions (FR). Each V_{H} and V_{L} can be composed of three CDRs and four FRs arranged from amino-terminus to carboxy-terminus in the following order: FR₁, CDR₁, FR₂, CDR₂, FR₃, CDR₃, and FR4. The constant regions of the antibodies may mediate the binding of the immunoglobulin to host tissues or factors, including various cells of the immune system (e.g., effector cells) and the first component (C1 q) of the classical complement system. The antibodies can be of any isotype (e.g., IgG, IgE, IgM, IgD, IgA and IgY), class (e.g., lgG₁, lgG₂, lgG₃, lgG₄, lgA₁ and lgA₂), subclass or modified version thereof. Antibodies may include a complete immunoglobulins or fragments thereof. An antibody fragment can refer to one or more fragments of an antibody that retain the ability to specifically bind to a tumor-associated protein. In addition, aggregates, polymers, and conjugates of immunoglobulins or their fragments can be used where appropriate so long as binding affinity for a particular molecule is maintained. Examples of antibody fragments include a Fab fragment, a monovalent fragment consisting of the V_{L}, V_{H}, C_{L} and C_{H1} domains; a F(ab)₂ fragment, a bivalent fragment comprising two Fab fragments linked by a disulfide bridge at the hinge region; an Fd fragment consisting of the V_{H} and C_{H1} domains; an Fv fragment consisting of the V_{L} and V_{H} domains of a single arm of an antibody; a single domain antibody (dAb) fragment (Ward et al., (1989) Nature 341:544-46), which consists of a V_{H} domain; and an isolated CDR and a single chain Fragment (scFv) in which the V_{L} and V_{H} regions pair to form monovalent molecules (known as single chain Fv (scFv); See, e.g., Bird et al., (1988) Science 242:423-26; and Huston et al., (1988) PNAS 85:5879-83). Thus, antibody fragments include Fab, F(ab)₂, scFv, Fv, dAb, and the like. Although the two domains V_{L} and V_{H} are coded for by separate genes, they can be joined, using recombinant methods, by an artificial peptide linker that enables them to be made as a single protein chain. Such single chain antibodies include one or more antigen binding moieties. These antibody fragments can be obtained using conventional techniques known to those of skill in the art, and the fragments can be screened for utility in the same manner as are intact antibodies. Antibodies can be human, humanized, chimeric, isolated, dog, cat, donkey, sheep, any plant, animal, or mammal. In some aspects, an antibody can be produced by as a result of a disease, disorder, or a condition.

Complexes

The methods provided herein can comprise forming complexes. A complex can refers to an association between at least two moieties (e.g. chemical or biochemical) that have an affinity for one another. The methods provided herein comprise forming a complex between a tumor-associated protein and an antibody, such as an autoantibody. In some embodiments, the methods comprise forming a complex between a tumor-associated protein and a single autoantibody. In some embodiments, the methods comprise forming a complex between a tumor-associated protein and a complex of two or more antibodies. In some embodiments, the methods can comprise forming a complex between a tumor-associated protein and a complex of two or more antibodies. In some embodiments, the methods comprise forming a complex between two or more tumor-associated proteins and a complex of two or more antibodies. In some embodiments, the methods comprise forming a complex between a first complex comprising a tumor-associated protein and another moiety (e.g., a polypeptide, polynucleotide, or small molecule) and an antibody. In some embodiments, the methods comprise forming a complex between a first complex comprising a tumor-associated protein and another moiety (e.g., a polypeptide, polynucleotide, or small molecule) and a second complex comprising two or more antibodies. For example, complexes can be formed between a tumor-associated protein coupled to a solid support, and an antibody to a tumor-associated protein.

Detection Methods

Detection methods for detecting tumor-associated protein-antibody complexes can include photometric and non-photometric means. In some embodiments, such methods process includes a method to detect and measure absorbance, fluorescence, phosphorescence, refractive index, polarization or light scattering. These include direct and/or indirect means to measure such parameters. Methods involving fluorescence include fluorescent tagging in immunological methods such as ELISA or sandwich assay. Methods involving refractive index include surface Plasmon resonance (SPR), grating coupled methods (e.g. sensors uniform grating couplers, wavelength-interrogated optical sensors (WIOS) and chirped grating couplers), resonant minor and interferometric techniques. Methods involving polarization include ellipsometry. Light scattering methods may also be used. Other means for tagging and/or separating and/or detecting can also include magnetic means. Magnetic resonance imaging, gas phase ion spectrometry, MRI may all be used.

Non-photometric methods of detection include, without limitation, magnetic resonance imaging, gas phase ion spectrometry, atomic force microscopy and multipolar coupled resonance spectroscopy. Magnetic resonance imaging (MRI) is based on the principles of nuclear magnetic resonance (NMR), a spectroscopic technique used by scientists to obtain microscopic chemical and physical information about molecules. Gas phase ion spectrometers include mass spectrometers, ion mobility spectrometers and total ion current measuring devices.

Mass spectrometers measure a parameter which can be translated into mass-to-charge ratios of ions. Generally, ions of interest bear a single charge, and mass-to-charge ratios are often simply referred to as mass. Mass spectrometers include an inlet system, an ionization source, an ion optic assembly, a mass analyzer, and a detector. Several different ionization sources have been used for desorbing and ionizing analytes from the surface of a support or biochip in a mass spectrometer. Such methodologies include laser desorption/ionization (MALDI, SELDI), fast atom bombardment, plasma desorption, and secondary ion mass spectrometers. In such mass spectrometers the inlet system comprises a support interface capable of engaging the support and positioning it in interrogatable relationship with the ionization source and concurrently in communication with the mass spectrometer, e.g., the ion optic assembly, the mass analyzer and the detector. Solid supports for use in bioassays that have a generally planar surface for the capture of targets and adapted for facile use as supports with detection instruments are generally referred to as biochips.

Analysis of the data generated typically involves quantification of a signal due to the detected antibody versus a control or reference. The data can be analyzed by any suitable means. Computers and computer programs may be utilized to generate and analyze the data. Beads and/or other supports may be computer coded or coded for identification purposes. Data analysis includes analysis of signal strength under the particular conditions of the assay or detection method. Tumor-associated proteins, antibodies, reference moieties and/or secondary detection moieties may be labeled or radio-labeled or tagged with a detectable moiety. One of ordinary skill in the art can also determine, pursuant to the methods described herein, the presence of false positives or other hits that are or may be found in control samples to account for and/or remove such hits and one of ordinary skill in the art, pursuant to the methods described herein, can continue the process of determining or finding disease associated biomarkers in subject samples having any disease or condition. The detection of such hits, in all cases, can be accomplished by

In some embodiments, detecting a binding of a moiety can comprise sequencing the moiety. In some embodiment, sequencing can be performed by nanopore sequencing. For example, Oxford nanopore sequencing. Nanopores may be used to sequence, a sample, a small portion (such as one full gene or a portion of one gene), a substantial portion (such as multiple genes or multiple chromosomes), or the entire genomic sequence of an individual. Nanopore sequencing technology may be commercially available or under development from Sequenom (San Diego, Calif.), Illumina (San Diego, Calif.), Oxford Nanopore Technologies LTD (Kidlington, United Kingdom), and Agilent Laboratories (Santa Clara, Calif.). Nanopore sequencing methods and apparatus are have been described in the art and for example are provided in U.S. Pat. No. 5,795,782, herein incorporated by reference in its entirety.

Nanopore sequencing can use electrophoresis to transport a sample through a pore. A nanopore system may contain an electrolytic solution such that when a constant electric field is applied, an electric current can be observed in the system. The magnitude of the electric current density across a nanopore surface may depend on the nanopore's dimensions and the composition of the sample that is occupying the nanopore. During nanopore sequencing, when a sample approaches and or goes through the nanopore, the samples cause characteristic changes in electric current density across nanopore surfaces, these characteristic changes in the electric current enables identification of the sample. Nanopores used herein may be solid-state nanopores, protein nanopores, or hybrid nanopores comprising protein nanopores or organic nanotubes such as carbon or graphene nanotubes, configured in a solid-state membrane, or like framework. In some embodiments, nanopore sequencing can be biological, a solid state nanopore or a hybrid biological/solid state nanopore.

In some instances, a biological nanopore can comprise transmembrane proteins that may be embedded in lipid membranes. In some embodiments, a nanopore described herein may comprise alpha hemolysin. In some embodiments, a nanopore described herein may comprise Mycobacterium smegmatis porn.

Solid state nanopores do not incorporate proteins into their systems. Instead, solid state nanopore technology uses various metal or metal alloy substrates with nanometer sized pores that allow samples to pass through. Solid state nanopores may be fabricated in a variety of materials including but not limited to, silicon nitride (Si3N4), silicon dioxide (Si02), and the like. In some instances, nanopore sequencing may comprise use of tunneling current, wherein a measurement of electron tunneling through bases as sample (ssDNA) translocates through the nanopore is obtained. In some embodiments, a nanopore system can have solid state pores with single walled carbon nanotubes across the diameter of the pore. In some embodiments, nanoelectrodes may be used on a nanopore system described herein. In some embodiments, fluorescence can be used with nanopores, for example solid state nanopores and fluorescence. For example, In such a system the fluorescence sequencing method converts each base of a sample into a characteristic representation of multiple nucleotides which bind to a fluorescent probe strand-forming dsDNA (were the sample comprises DNA). Where a two color system is used, each base is identified by two separate fluorescence, and will therefore be converted into two specific sequences. Probes may consist of a fluorophore and quencher at the start and end of each sequence, respectively. Each fluorophore may be extinguished by the quencher at the end of the preceding sequence. When the dsDNA is translocating through a solid state nanopore, the probe strand may be stripped off, and the upstream fluorophore will fluoresce.

In some embodiments, a 1-100 nm channel or aperture may be formed through a solid substrate, usually a planar substrate, such as a membrane, through which an analyte, such as single stranded DNA, is induced to translocate. In other embodiments, a 2-50 nm channel or aperture is formed through a substrate; and in still other embodiments, a 2-30 nm, or a 2-20 nm, or a 3-30 nm, or a 3-20 nm, or a 3-10 nm channel or aperture if formed through a substrate.

In some embodiments, nanopores used in connection with the methods and devices of the invention are provided in the form of arrays, such as an array of clusters of nanopores, which may be disposed regularly on a planar surface. In some embodiments, clusters are each in a separate resolution limited area so that optical signals from nanopores of different clusters are distinguishable by the optical detection system employed, but optical signals from nanopores within the same cluster cannot necessarily be assigned to a specific nanopore within such cluster by the optical detection system employed means for detecting the binding of a tumor-associated protein to an antibody.

Binding Assays

Binding assays can also be useful, e.g., for identifying disease related antibodies that interact with the tumor-associated proteins described herein. For example, antibodies or other molecules that bind proteins of the invention can be identified in binding assays. Binding assays can involve, but are not limited to, use of isolated polypeptides, crude extracts, or cell-based assays. In some embodiments the assays described herein can be used to a) identify subjects whose have a first disease or a second disease; (b) assess the impact of a disease therapy; and (c) monitor disease progression.

Binding assays can involve contacting a protein with a sample comprising an antibody and allowing sufficient time for the molecule and test agents to form a binding complex. Any binding complexes formed can be detected using any of a number of established analytical techniques. Binding assays include, but are not limited to, methods that measure co-precipitation or co-migration on non-denaturing SDS-polyacrylamide gels, co-migration on Western blots, enzyme linked immunosorbent assay (ELISA), radioimmunoassay (RIA), immunoradiometric assay, fluoroimmunoassay, chemiluminescent assay, bioluminescent assay, FACS, FRET. The steps of various useful immunodetection methods have been described in the scientific literature, such as, e.g., Doolittle and Ben-Zeev (1999), Gulbis and Galand (1993), De Jager et al. (1993), and Nakamura et al. (1987). (see, e.g., Bennet, J. P. and Yamamura, H. I. (1985) “Neurotransmitter, Hormone or Drug Receptor Binding Methods,” in Neurotransmitter Receptor Binding (Yamamura, H. L, et al, eds.), pp. 61-89. Other binding assays involve the use of mass spectrometry or NMR techniques to identify target analyte bound the antibody or displacement of labeled substrates. The antibodies used in these assays can be naturally expressed, cloned or synthesized. [108] In addition, mammalian or yeast two-hybrid approaches (see, e.g., Bartel, P. L. et. al. Methods Enzymol, 254:241 (1995)) can be used to identify polypeptides or other molecules that interact or bind to the polypeptide when expressed together in a host cell. U.S. Pat. Nos. 3,817,837, 3,850,752, 3,939,350, 3,996,345, 4,277,437, 4,275,149 and 4,366,241 are hereby incorporated by reference in its entirety.

An example chemiluminescence assay protocol is as follows: Each purified protein was coupled with Magnetic bead by a ratio of 4 μg/mg. Then, 50 μl magnetic bead coupling with purified protein (0.4 mg/ml, diluted with Buffer-10) was mixed with 50 μl serum sample (1:100 diluted with Buffer-06), and incubated for 15 min at 37° C.; Beads were washed 3 times with PBST; 100 μl ALP-labeled anti-human IgG monoclonal antibody (1:10000 diluted with Buffer-03, Jackson immune 109-036-008)/or 100 μl ALP-labeled anti-human IgA monoclonal antibody (1:10000 diluted with Buffer-03, Jackson immune 109-036-011) was added and incubated for 15 min at 37° C.; Beads were washed 3 times with PBST; Chemiluminescence was measured in ChemLite 2000-8# (Chemiluminescence platform of CapitalBio mc). Buffer-10, Buffer-06 and Buffer-03 were provided by CapitalBio inc.

ELISA

Immunoassays, in their most simple and direct sense, are binding assays. Certain immunoassays finding particular use in the present invention are various types of enzyme linked immunosorbent assays (ELISAs) and radioimmunoassays (RIA) known in the art. In one exemplary ELISA, the tumor-associated protein of the invention are immobilized onto a selected surface, such as a well in a polystyrene microtiter plate. Then, a test composition suspected of containing the antibody is added to the wells. After binding and washing to remove non-specifically bound complexes, the bound antibody may be detected. Detection may be achieved by the addition of another ligand linked to a detectable label. This type of assay is analogous to a simple “sandwich ELISA” except that binding of the labeled agent is direct at the Fab portion of the bound antibody. Detection may also be achieved by the addition of a labeled antibody that binds any bound antibody, e.g., that recognizes the Fc portion of the bound antibody. Optionally, this antibody is not labeled, and is followed by the addition of a second antibody that has binding affinity for the first antibody, with the second antibody being linked to a detectable label.

In another exemplary ELISA, the samples suspected of containing the antibodies are immobilized onto a well surface and then contacted with labeled tumor-associated proteins of the present invention. After binding and washing to remove non-specifically bound immune complexes, the bound labeled ligands are detected. Alternatively, the ligands are not labeled and can be detected against an artificial antibody (non-sample) that is selected for specific binding the tumor-associated protein of choice, this second would be linked to a detectable label, thereby permitting detection.

Irrespective of the format employed, ELISAs have certain features in common, such as coating, incubating and binding, washing to remove non-specifically bound species, and detecting the bound immune complexes. These are described below.

In coating a plate with either tumor-associated protein or antibody, one can generally incubate the wells of the plate with a solution of the protein or antibody, either overnight or for a specified period of hours. In certain aspects, the plate can be blocked using a bacterial lysate, such as an E. coli lysate (See Example 1). The wells of the plate can then be washed to remove incompletely adsorbed material. Any remaining available surfaces of the wells can then be coated with a non-specific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a target analyte or antibody to the well, coating with a non-reactive material to reduce background, and washing to remove unbound material, the immobilizing surface can be contacted with a biological sample or tumor-associated protein to be tested under conditions effective to allow immune complex formation. Detection of the immune complex then requires a labeled secondary binding ligand or antibody, or a secondary binding ligand or antibody in conjunction with a labeled tertiary antibody (with specificity either for the Fc region of the antibody or the ligand).

Under conditions effective to allow immune complex (antigen/antibody) formation means that the conditions can include diluting the antigens and/or antibodies with solutions such as BSA, bovine gamma globulin (BGG) or phosphate buffered saline (PBS)/Tween. These added agents can assist in the reduction of nonspecific background.

The suitable conditions can also mean that the incubation is at a temperature or for a period of time sufficient to allow effective binding. Incubation steps can be from about 1 to 2 to 4 to 6 to 24 to about 48 hours or so, at temperatures on the order of about 20° C. to about 37° C. In some embodiments, about 21° C., 22° C., 23° C., 24° C., 25° C., 26° C., 27° C., 28° C., 29° C., 30° C., 31° C., 32, ° C., 33° C., 34° C., 35° C., 36° C., or about 37° C. or may be overnight at about 2° C., 3° C., 4° C., 5° C., 6° C., 7° C. or so.

Following incubation steps in an ELISA, the contacted surface can be washed so as to remove non-complexed material. In some embodiments, the washing procedure includes washing with a solution such as PBS/Tween, or borate buffer. Following the formation of specific immune complexes between the test sample and the originally bound material, and subsequent washing, the occurrence of even minute amounts of immune complexes may be determined.

Detection may utilize an enzyme that can generate color development upon incubating with an appropriate chromogenic substrate. Thus, for example, one can desire to contact or incubate the first and second immune complex with a urease, glucose oxidase, alkaline phosphatase or hydrogen peroxidase-conjugated antibody or ligand for a period of time and under conditions that favor the development of further immune complex formation (e.g., incubation for about 1, 2, 3, 4, 5, 6, 7, 8, 9, or about 10 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label.

Quantification can be achieved by measuring the degree of color generated, e.g., using a visible spectra spectrophotometer.

An example ELISA protocol is as follows: 100 μl of each purified protein (0.5 ng/μl, 50 ng/well) was coated onto individual wells of an ELISA plate, and incubated overnight at 4° C.; Washed 3 times with PBST (0.2% Tween-20), 300 μl/well; Blocked with 3% BSA (300 μl/well, Amresco) for 1 h at 37° C.; Washed 1 time with PBST, 300 μl/well; 100 μl of each serum sample (1:500 diluted with PBST) was added to the well, and incubated for 1 h at 37° C.; Washed 4 times with PBST, 300 μl/well; 100 μl of HRP-labeled anti-human IgG monoclonal antibody (1:50000 diluted with PBST, Jackson immune 109-036-008) was added to the well; Washed 4 times with PBST, 300 μl/well; 50 W TMB and 50 μl H₂O₂ were added, respectively, and incubated for 6-8 min at 37° C.; Reaction was terminated by addition of 50 μl of 2 N H₂SO₄, and immunoreactivity was measured by reading the A₄₅₀. 100 μl of each purified protein (0.5 ng/μl, 50 ng/well) was coated onto individual wells of an ELISA plate, and incubated overnight at 4° C.; Washed 3 times with PBST (0.2% Tween-20), 300 μl/well; Blocked with 3% BSA (300 μl/well, Amresco) for 1 h at 37° C.; Washed 1 time with PBST, 300 μl/well; 100 μl of each serum sample (1:100 diluted with PBST) was added to the well, and incubated for 1 h at 37° C.; Washed 4 times with PBST, 300 μl/well; 100 μl of HRP-labeled anti-human IgA monoclonal antibody (1:20000 diluted with PBST, Jackson immune 109-036-011) was added to the well; Washed 4 times with PBST, 300 μl/well; 50 μl TMB and 50 μl H₂O₂ were added, respectively, and incubated for 12-15 min at 37° C.; Reaction was terminated by addition of 50 μl of 2 N H₂SO₄, and immunoreactivity was measured by reading the A₄₅₀.

Förster Resonance Energy Transfer (FRET)

FRET is a phenomenon in which the excited-state energy in one molecule (called the donor) is transferred to another molecule by a radiationless coupling. This mechanism was first correctly described by Förster, and differs from other types of energy transfer, such as electron sharing (Dexter) or trivial transfer (emission of a photon from the donor and reabsorption by the acceptor). The Dexter mechanism requires the two molecules to be in physical contact, while trivial transfer is a very low probability. In contrast, the Förster mechanism exhibits a high probability when the two molecules are within the Förster radius, which is defined for any given pair of fluorophores.

The overall FRET efficiency depends on the Förster radius, and is determined by several factors and is directly related to the amount of overlap between the absorption spectra of the acceptor molecule and the emission spectra of the donor molecule. The amount of FRET also depends on the alignment of the donor and acceptor molecules, although most biological systems are not rigidly aligned. The FRET efficiency is also affected by the ability of the acceptor molecule to absorb light, as indicated by its molar extinction coefficient, and the overall stability of the excited state of the donor molecule, as indicated by the probability that absorption will lead to fluorescence (quantum yield) and the lifetime of the excited state.

FRET between two different fluorophores can be assayed by several methods: looking at the change in color of the fluorescence, measuring the fluorescence lifetime of the donor, examining the changes upon photo bleaching either the donor or acceptor, or by measuring the fluorescence polarization of the acceptor. Regardless of the approach, most of these assays share common features of the instrumentation.

The types of microscopes used to measure FRET can be suitably selected depending on the purpose. In some embodiments, where frequent observations are necessary for monitoring a time course of the changing, conventional incident-light fluorescent microscope can be used. In some embodiments, where resolution is to be increased as in the case where detailed intercellular localization is to be monitored, confocal laser microscope can be used. As a microscope system, an inverted microscope can be used for most live cell measurements in view of keeping the physiological state of cell and preventing contamination. When an upright microscope is used, a water immersion lens can be used in the case of using lens of high power.

The filter set can be suitably selected depending on the fluorescent wave length of the fluorescent protein. For the observation of GFP, a filter with excitation light of about 470-490 nm and fluorescent light of about 500-520 nm can be used. For the observation of YFP, a filter with excitation light of about 490-510 nm and fluorescent light of about 520-550 nm can be used. For the observation of CFP, it is preferred to use a filter with excitation light of about 425 nm and fluorescent light of about 460-500 nm. Moreover, when time course observation is carried out in living cells by using a fluorescent microscope, the cells can be photographed in a short period, and therefore a high sensitive cooled CCD camera can be used. By using a cooled CCD camera, thermal noise can be decreased by cooling CCD, and weak fluorescent image can be clearly acquired by exposure of short period. Confocal microscopes can also be used for live cell imaging, as long as care is taken to minimize the exposure times.

In a similar manner, any ligand may be screened on the beads or supports using the processes described herein. These ligands include, in addition to peptoids or peptides, nucleic acid oligomers, polysaccharides, small molecules and/or any combination thereof which can be built into libraries and, under the conditions recited herein, used to screen biological fluid.

In some embodiments, detecting may comprise radio immunoassay (“RIA”), fluorescence immunoassay (“FIA”), enzyme-linked immunosorbent assay (“ELISA”), Western blot, flow cytometry, Forster resonance energy transfer (“FRET”), or surface plasmon resonance.

Methods of Use

The methods, kits, and compositions described herein can be used for numerous applications, including identification of binding partners, determination of affinities of autoantibodies to tumor-associated proteins, determination of specificities of autoantibodies to tumor-associated proteins, quantification of tumor-associated proteins in a sample, quantification of autoantibodies in a sample, quantification of binding events, identification of a disease, identification of biomarkers of a disease or condition, drug discovery, molecular biology, immunology and toxicology. Arrays can be used for large scale binding assays in numerous diagnostic and screening applications. These methods of use include, but are not limited to, high-content, high-throughput assays for screening for antibodies that interact with tumor-associated proteins. Additional methods of use include medical diagnostic, proteomic, and biosensor assays. The multiplexed measurement of quantitative variation in levels of large numbers of tumor-associated proteins allows the recognition of patterns defined by several to many different tumor-associated proteins. The multiplexed identification of large numbers of interactions between tumor-associated proteins and autoantibodies allows for the recognition of binding and interaction patterns defined by several to many different interactions between tumor-associated proteins and autoantibodies.

The assays used with the arrays of the presently disclosed subject matter may be direct, noncompetitive assays or indirect, competitive assays. In the noncompetitive method, the affinity for a tumor-associated protein to an antibody can be determined directly. In this method, the tumor-associated protein can be directly exposed to an antibody. The antibody may be labeled or unlabeled.

A label refers to a molecule that, when attached to another molecule provides or enhances a means of detecting the other molecule. A signal emitted from a label can allow detection of the molecule or complex to which it is attached, and/or the label itself. For example, a label can be a molecular species that elicits a physical or chemical response that can be observed or detected by the naked eye or by means of instrumentation such as, without limitation, scintillation counters, colorimeters, UV spectrophotometers and the like. Labels include but are not limited to, radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. A fluorescence or fluorescent label or tag emits detectable light at a particular wavelength when excited at a different wavelength. A radiolabel or radioactive tag emits radioactive particles detectable with an instrument such as, without limitation, a scintillation counter. Other signal generation detection methods include: chemiluminescence detection, electrochemiluminescence detection, Raman energy detection, colorimetric detection, hybridization protection assays, and mass spectrometry.

If an antibody is labeled, the methods of detection could include fluorescence, phosphorescence luminescence, radioactivity, and the like. If an antibody is unlabeled, the detection of binding can be based on a change in some physical property of the tumor-associated protein. Such physical properties could include, for example, a refractive index or electrical impedance. The detection of binding of unlabeled antibody could include, for example, mass spectroscopy. The detection of binding can also be carried out using sandwich assays, in which after the initial binding, the array is incubated with a second solution containing molecules such as labeled antibodies that have an affinity for the autoantibody bound to the tumor-associated protein, and the amount of binding is determined based on the amount of binding of the labeled antibodies to the autoantibody. The detection of binding can be carried out using a displacement assay in which after the initial binding of a labeled moiety, the array is incubated with a second solution containing unlabeled binding moiety. The binding capability and the amount of binding of the binding moiety are determined based on the decrease in number of the pre-bound labeled moieties in the tumor-associated proteins.

Also disclosed herein is a method of determining a relative binding affinity of an antibody for a tumor-associated protein.

The relative binding affinity of an antibody for a tumor-associated protein may be measured by measuring or counting the coupled product and/or amplified products thereof by using any suitable method known in the art.

A variety of different agents may be screened by the above methods. Candidate agents encompass numerous chemical classes including, but not limited to, peptides, polynucleotides, and organic molecules (e.g., small organic compounds having a molecular weight of more than 50 and less than about 2,500 Daltons). Candidate agents can comprise functional groups for structural interaction with tumor-associated proteins, such as hydrogen bonding, and can include at least one or at least two of an amine, carbonyl, hydroxyl or carboxyl group. The candidate agents can comprise cyclical carbon or heterocyclic structures and/or aromatic or polyaromatic structures substituted with one or more functional groups. Candidate agents can be biomolecules including peptides, saccharides, fatty acids, steroids, purines, pyrimidines, derivatives, structural analogs or combinations thereof. Candidate agents can be obtained from a wide variety of sources including libraries of synthetic or natural compounds. For example, numerous means are available for random and directed synthesis of a wide variety of organic compounds and biomolecules, including expression of randomized polynucleotides and polypeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, acidification, etc. to produce structural analogs. Agents identified find uses in a variety of methods, including methods of modulating the activity of a tumor-associated protein, and conditions related to the presence, activity, and/or interactions of a tumor-associated protein

Diagnostics

The methods and apparatus disclosed herein can be used to screen for various diseases or conditions, including an alteration in the state of the body or of some of the organs, interrupting or disturbing the performance of the functions and/or causing symptoms such as discomfort, dysfunction, distress, or even death to the person afflicted or those in contact with a person. A disease or condition can also include a distemper, ailing, ailment, malady, disorder, sickness, illness, complain, interdisposition and/or affectation.

For example, samples containing antibodies from a diseased animal can be simultaneously screened for the antibodies' ability to interact with tumor-associated proteins on an array. These interactions can be compared to those of samples from individuals that are not in a disease state, not presenting symptoms of persons in the disease state, or presenting symptoms of the disease state. For example, the levels of tumor-associated proteins in samples from a diseased animal can be simultaneously determined. These levels can be compared to those of samples from individuals that are not in a disease state, not presenting symptoms of persons in the disease state, or presenting symptoms of the disease state.

The methods, kits, and compositions described herein can be used in medical diagnostics, drug discovery, molecular biology, immunology and toxicology. Arrays can be used for large scale binding assays in numerous diagnostic and screening applications. The multiplexed measurement of quantitative variation in levels of large numbers of tumor-associated proteins (e.g. proteins) allows the recognition of patterns defined by several to many different tumor-associated proteins. The multiplexed identification of large numbers of interactions between tumor-associated proteins and antibodies allows for the recognition of binding and interaction patterns defined by several to many different interactions between tumor-associated proteins and antibodies. Many physiological parameters and disease-specific patterns can be simultaneously assessed. One embodiment involves the separation, identification and characterization of proteins present in a biological sample. For example, by comparison of disease and control samples, it is possible to identify disease specific tumor-associated proteins. These tumor-associated proteins can be used as targets for drug development or as molecular markers of disease.

Detection a level of one or more tumor-associated proteins or detection of interactions between autoantibodies and tumor-associated proteins can lead to a medical diagnosis. The sample can be a sample from a subject with a condition or disease. For example, a sample can be a diseased tissue or cell, such as a breast cancer, ovarian cancer, lung cancer (including SCLC or NSCLC), colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer tissue or cell. The sample can be from a subject with a disease or condition such as a cancer, inflammatory disease, immune disease, autoimmune disease, cardiovascular disease, neurological disease, infectious disease, metabolic disease, or a perinatal condition. For example, the disease or condition can be a tumor, neoplasm, or cancer. The cancer can be, but is not limited to, breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer. The colorectal cancer can be CRC Dukes B or Dukes C-D. The hematological malignancy can be B-Cell Chronic Lymphocytic Leukemia, B-Cell Lymphoma-DLBCL, B-Cell Lymphoma-DLBCL-germinal center-like, B-Cell Lymphoma-DLBCL-activated B-cell-like, or Burkitt's lymphoma. The disease or condition can also be a premalignant condition, such as Barrett's Esophagus. The disease or condition can also be an inflammatory disease, immune disease, or autoimmune disease. For example, the disease may be inflammatory bowel disease (IBD), Crohn's disease (CD), ulcerative colitis (UC), pelvic inflammation, vasculitis, psoriasis, diabetes, autoimmune hepatitis, Multiple Sclerosis, Myasthenia Gravis, Type I diabetes, Rheumatoid Arthritis, Psoriasis, Systemic Lupus Erythematosis (SLE), Hashimoto's Thyroiditis, Grave's disease, Ankylosing Spondylitis Sjogrens Disease, CREST syndrome, Scleroderma, Rheumatic Disease, organ rejection, Primary Sclerosing Cholangitis, or sepsis. The disease or condition can also be a cardiovascular disease, such as atherosclerosis, congestive heart failure, vulnerable plaque, stroke, or ischemia. The cardiovascular disease or condition can be high blood pressure, stenosis, vessel occlusion or a thrombotic event. The disease or condition can also be a neurological disease, such as Multiple Sclerosis (MS), Parkinson's Disease (PD), Alzheimer's Disease (AD), schizophrenia, bipolar disorder, depression, autism, Prion Disease, Pick's disease, dementia, Huntington disease (HD), Down's syndrome, cerebrovascular disease, Rasmussen's encephalitis, viral meningitis, neuropsychiatric systemic lupus erythematosus (NPSLE), amyotrophic lateral sclerosis, Creutzfeldt-Jacob disease, Gerstmann-Straussler-Scheinker disease, transmissible spongiform encephalopathy, ischemic reperfusion damage (e.g. stroke), brain trauma, microbial infection, or chronic fatigue syndrome. The condition may also be fibromyalgia, chronic neuropathic pain, or peripheral neuropathic pain. The disease or condition may also be an infectious disease, such as a bacterial, viral or yeast infection. For example, the disease or condition may be Whipple's Disease, Prion Disease, cirrhosis, methicillin-resistant Staphylococcus aureus, HIV, hepatitis, syphilis, meningitis, malaria, tuberculosis, or influenza. The disease or condition can also be a perinatal or pregnancy related condition (e.g. preeclampsia or preterm birth), zika virus, dengue fever, flavivirus, or a metabolic disease or condition, such as a metabolic disease or condition associated with iron metabolism.

Specificity and Sensitivity

Methods herein can further comprise determining the effectiveness of a given biomarker (e.g., tumor-associated protein or antibody) or a given group of biomarkers. Parameters to be measured include those described in Fischer et al., Intensive Care Med. 29: 1043-51, 2003, which is incorporated herein in its entirety. These parameters include sensitivity and specificity, predictive values, likelihood ratios, diagnostic odds ratios, and receiver operating characteristic (ROC) curve areas. One or a group of effective biomarkers can exhibit one or more of the following results on these various parameters: at least 75% sensitivity, combined with at least 75% specificity; ROC curve area of at least 0.7, at least 0.8, at least 0.9, or at least 0.95; and/or a positive likelihood ratio (calculated as sensitivity/(1−specificity)) of at least 5, at least 10, or at least 20, and a negative likelihood ratio (calculated as (1-sensitivity)/specificity) of less than or equal to 0.3, less than or equal to 0.2, or less than or equal to 0.1. The ROC areas can be calculated and used in determining the effectiveness of a biomarker as described in US Patent Application Publication No. 2013/0189243, which is incorporated herein in its entirety.

Methods, devices and kits provided herein can assess a condition (e.g., cancer) in a subject with high specificity and sensitivity. In some embodiments, the cancer can be lung cancer. As used herein, the term “specificity” can refer to a measure of the proportion of negatives that are correctly identified as such (e.g., the percentage of healthy people who are correctly identified as not having the condition). As used herein, the term “sensitivity” can refer to a measure of the proportion of positives that are correctly identified as such (e.g., the percentage of sick people who are correctly identified as having the condition). Methods, devices and kits provided herein can assess a condition in a subject with a specificity of at least about 75%, 80%, 85%, 90%, 95%, 96%, 97%, 98%, 99%, or 100%. Methods, devices and kits provided herein can assess a condition in a subject with a sensitivity of at least about 10%, 15%, 20%, 25%, 30%, 35%, 40%, 45%, 50%, 55%, 60%, 70%, 75%, 80%, 85%, 90%, 95%, 96%, 97%, 98%, 99%, or 100%. Methods, devices and kits provided herein can assess a condition in a subject with a specificity of at least about 70% and a sensitivity of at least about 70%, a specificity of at least about 75% and a sensitivity of at least about 75%, a specificity of at least about 80% and a sensitivity of at least about 80%, a specificity of at least about 85% and a sensitivity of at least about 85%, a specificity of at least about 90% and a sensitivity of at least about 90%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of about 100% a sensitivity of about 100%.

Methods of assessing a condition in a subject herein can achieve high specificity and sensitivity based on the expression of various numbers of biomarkers. In some cases, the methods of assessing a condition in a subject can achieve a specificity of at least about 70% and a sensitivity of at least about 70%, a specificity of at least about 75% and a sensitivity of at least about 75%, a specificity of at least about 80% and a sensitivity of at least about 80%, a specificity of at least about 85% and a sensitivity of at least about 85%, a specificity of at least about 90% and a sensitivity of at least about 90%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of 100% a sensitivity of 100% based on the expression of no more than 2, 3, 4, 5, 6, 7, 8, 9, or 10 biomarkers. In some cases, the methods, devices and kits of assessing a condition in a subject can achieve a specificity of at least about 92% and a sensitivity of at least about 92%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of about 100% and a sensitivity of about 100% based on the expression of two biomarkers.

In some cases, the methods of assessing a condition in a subject can comprise measuring or detecting the expression of two or more of any antibody or tumor-associated protein disclosed herein for example (p53, ETHE1, CTAG1A, C1QTNF1, TEX264, CLDN2, NSG1, HRAs, CKAP2, DPP4, CAB39, or STRA13, LIMS1, RBPJ1, RBPJ2, RBPJ3, MAK16, CACLOCO2, SDCBP, LGALS8, DMRT2, SS18, YP021, APH1A, TOM1L2, PIP4K2C, NUMBL, NUDT14, RALGDS, SPN, RPLP1, DCLK1, SULT2B1, C14orf37, MAGEA8, DDX4, BCL7A, NOL3, SSBP4, TXNDC2, FIGNL2, JAKMIP2, KCNRG, N4BP1, ACPP, TRIM33, MTERF4, TTC1, MAGEC2, or a fragment of any of the above) and the method can achieve a specificity of at least 50% and a sensitivity of at least 50%, a specificity of at least 55% and a sensitivity of at least 55%, a specificity of at least 60% and a sensitivity of at least 60%, a specificity of at least 65% and a sensitivity of at least 65%, a specificity of at least 70% and a sensitivity of at least 70%, a specificity of at least 75% and a sensitivity of at least 75%, a specificity of at least 80% and a sensitivity of at least 80%, a specificity of at least 85% and a sensitivity of at least 85%, a specificity of at least 90% and a sensitivity of at least 90%, a specificity of at least 92% and a sensitivity of at least 92%, a specificity of at least 95% and a sensitivity of at least 95%, a specificity of at least 96% and a sensitivity of at least 96%, a specificity of at least 97% and a sensitivity of at least 97%, a specificity of at least 98% and a sensitivity of at least 98%, a specificity of at least 99% and a sensitivity of at least 99%, or a specificity of 100% and a sensitivity of 100%.

Assessing lung cancer can comprise distinguishing a subject with lung cancer from a healthy subject, or a subject with a second disease. In some embodiments, the second disease is a second cancer. In some embodiments, the second disease is a non-cancer pulmonary disease. In some embodiments, the non-cancer pulmonary disease is benign lung lesions (LBL), pneumonia, chronic obstructive pulmonary disease (COPD), or pulmonary tuberculosis. Methods, devices, and kits herein can achieve high specificity and sensitivity in distinguishing a subject with a disease disclosed herein for example lung cancer from a healthy subject, and distinguishing the subject with lung cancer from a subject with a second disease. For example, methods, devices, and kits herein can achieve a specificity of at least 92% and a sensitivity of at least 92%, a specificity of at least 95% and a sensitivity of at least 95%, a specificity of at least 96% and a sensitivity of at least 96%, a specificity of at least 97% and a sensitivity of at least 97%, a specificity of at least 98% and a sensitivity of at least 98%, a specificity of at least 99% and a sensitivity of at least 99%, or a specificity of 100% and a sensitivity of 100% in distinguishing a subject with lung cancer from a healthy subject, and meanwhile can achieve a specificity of at least 92% and a sensitivity of at least 92%, a specificity of at least 95% and a sensitivity of at least 95%, a specificity of at least 96% and a sensitivity of at least 96%, a specificity of at least 97% and a sensitivity of at least 97%, a specificity of at least 98% and a sensitivity of at least 98%, a specificity of at least 99% and a sensitivity of at least 99%, or a specificity of 100% and a sensitivity of 100% in distinguishing the subject with lung cancer from a subject with a second disease.

Supports/Substrates

The present disclosure provides substrates and methods of making substrates. The nature and geometry of a support or substrate can depend upon a variety of factors, including the type of array (e.g., one-dimensional, two-dimensional or three-dimensional). Generally, a substrate can be composed of any material which will permit coupling of a tumor-associated protein, which will not melt or otherwise substantially degrade under the conditions used to hybridize and/or denature nucleic acids. A substrate can be composed of any material which will permit coupling of a tumor-associated protein, and/or other moiety at one or more discrete regions and/or discrete locations within the discrete regions. A substrate can be composed of any material which permit washing or physical or chemical manipulation without dislodging a tumor-associated protein or antibody from the solid support.

Substrates can be fabricated by the transfer of tumor-associated protein onto the solid surface in an organized high-density format followed by coupling the tumor-associated protein thereto. The techniques for fabrication of a substrate of the invention include, but are not limited to, photolithography, ink jet and contact printing, liquid dispensing and piezoelectrics. The patterns and dimensions of arrays are to be determined by each specific application. The sizes of each tumor-associated protein spots may be easily controlled by the users.

A method of making a solid substrate can comprise contacting or coupling a tumor-associated protein to a discrete location.

A substrate may take a variety of configurations ranging from simple to complex, depending on the intended use of the array. Thus, a substrate can have an overall slide or plate configuration, such as a rectangular or disc configuration. A standard microplate configuration can be used. In some embodiments, the surface may be smooth or substantially planar, or have irregularities, such as depressions or elevations. For example, the substrates of the presently disclosed subject matter can include at least one surface on which a pattern of recombinant virion microspots can be coupled or deposited. In some instances, a substrate may have a rectangular cross-sectional shape, having a length of from about 10-200 mm, 40-150 mm, or 75-125 mm; a width of from about 10-200 mm, 20-120 mm, or 25-80 mm, and a thickness of from about 0.01-5.0 mm, 0.1-2 mm, or 0.2 to 1 mm.

A support may be organic or inorganic; may be metal (e.g., copper or silver) or non-metal; may be a polymer or nonpolymer; may be conducting, semiconducting or nonconducting (insulating); may be reflecting or nonreflecting; may be porous or nonporous; etc. A solid support as described above can be formed of any suitable material, including metals, metal oxides, semiconductors, polymers (particularly organic polymers in any suitable form including woven, nonwoven, molded, extruded, cast, etc.), silicon, silicon oxide, and composites thereof.

A number of materials (e.g., polymers) suitable for use as substrates (e.g., solid substrates) in the instant invention have been described in the art. Suitable materials for use as substrates include, but are not limited to, polycarbonate, gold, silicon, silicon oxide, silicon oxynitride, indium, tantalum oxide, niobium oxide, titanium, titanium oxide, platinum, iridium, indium tin oxide, diamond or diamond-like film, acrylic, styrene-methyl methacrylate copolymers, ethylene/acrylic acid, acrylonitrile-butadiene-styrene (ABS), ABS/polycarbonate, ABS/polysulfone, ABS/polyvinyl chloride, ethylene propylene, ethylene vinyl acetate (EVA), nitrocellulose, nylons (including nylon 6, nylon 6/6, nylon 6/6-6, nylon 6/9, nylon 6/10, nylon 6/12, nylon 11 and nylon 12), polyacrylonitrile (PAN), polyacrylate, polycarbonate, polybutylene terephthalate (PBT), poly(ethylene) (PE) (including low density, linear low density, high density, cross-linked and ultra-high molecular weight grades), poly(propylene) (PP), cis and trans isomers of poly(butadiene) (PB), cis and trans isomers of poly(isoprene), polyethylene terephthalate) (PET), polypropylene homopolymer, polypropylene copolymers, polystyrene (PS) (including general purpose and high impact grades), polycarbonate (PC), poly(epsilon-caprolactone) (PECL or PCL), poly(methyl methacrylate) (PMMA) and its homologs, poly(methyl acrylate) and its homologs, poly(lactic acid) (PLA), poly(glycolic acid), polyorthoesters, poly(anhydrides), nylon, polyimides, polydimethylsiloxane (PDMS), polybutadiene (PB), polyvinylalcohol (PVA), polyacrylamide and its homologs such as poly(N-isopropyl acrylamide), fluorinated polyacrylate (PFOA), poly(ethylene-butylene) (PEB), poly(styrene-acrylonitrile) (SAN), polytetrafluoroethylene (PTFE) and its derivatives, polyolefin plastomers, fluorinated ethylene-propylene (FEP), ethylene-tetrafluoroethylene (ETFE), perfluoroalkoxyethylene (PFA), polyvinyl fluoride (PVF), polyvinylidene fluoride (PVDF), polychlorotrifluoroethylene (PCTFE), polyethylene-chlorotrifluoroethylene (ECTFE), styrene maleic anhydride (SMA), metal oxides, glass, silicon oxide or other inorganic or semiconductor material (e.g., silicon nitride), compound semiconductors (e.g., gallium arsenide, and indium gallium arsenide), and combinations thereof.

Examples of well-known solid supports include polypropylene, polystyrene, polyethylene, dextran, nylon, amylases, glass, natural and modified celluloses (e.g., nitrocellulose), polyacrylamides, agaroses and magnetite. In some instances, the solid support can be silica or glass because of its great chemical resistance against solvents, its mechanical stability, its low intrinsic fluorescence properties, and its flexibility of being readily functionalized. In one embodiment, the substrate is glass, particularly glass coated with nitrocellulose, more particularly a nitrocellulose-coated slide (e.g., FAST slides).

A substrate may be modified with one or more different layers of compounds or coatings that serve to modify the properties of the surface in a desirable manner. For example, a substrate may further comprise a coating material on the whole or a portion of the surface of the substrate. In some embodiments, a coating material enhances the affinity of the tumor-associated protein or another moiety (e.g., a functional group) for the substrate. For example, the coating material can be nitrocellulose, silane, thiol, disulfide, or a polymer. When the material is a thiol, the substrate may comprise a gold-coated surface and/or the thiol comprises hydrophobic and hydrophilic moieties. When the coating material is a silane, the substrate comprises glass and the silane may present terminal moieties including, for example, hydroxyl, carboxyl, phosphate, glycidoxy, sulfonate, isocyanato, thiol, or amino groups. In an alternative embodiment, the coating material may be a derivatized monolayer or multilayer having covalently bonded linker moieties. For example, the monolayer coating may have thiol (e.g., a thioalkyl selected from the group consisting of athioalkyl acid (e.g., 16-mercaptohexadecanoic acid), thioalkyl alcohol, thioalkyl amine, and halogen containing thioalkyl compound), disulfide or silane groups that produce a chemical or physicochemical bonding to the substrate. The attachment of the monolayer to the substrate may also be achieved by non-covalent interactions or by covalent reactions.

After attachment to the substrate, the coating may comprise at least one functional group. Examples of functional groups on the monolayer coating include, but are not limited to, carboxyl, isocyanate, halogen, amine or hydroxyl groups. In one embodiment, these reactive functional groups on the coating may be activated by standard chemical techniques to corresponding activated functional groups on the monolayer coating (e.g., conversion of carboxyl groups to anhydrides or acid halides, etc.). Exemplary activated functional groups of the coating on the substrate for covalent coupling to terminal amino groups include anhydrides, N-hydroxysuccinimide esters or other common activated esters or acid halides, Exemplary activated functional groups of the coating on the substrate include anhydride derivatives for coupling with a terminal hydroxyl group; hydrazine derivatives for coupling onto oxidized sugar residues of the linker compound; or maleimide derivatives for covalent attachment to thiol groups of the linker compound. To produce a derivatized coating, at least one terminal carboxyl group on the coating can be activated to an anhydride group and then reacted, for example, with a linker compound. Alternatively, the functional groups on the coating may be reacted with a linker having activated functional groups (e.g., N-hydroxysuccinimide esters, acid halides, anhydrides, and isocyanates) for covalent coupling to reactive amino groups on the coating.

A substrate can contain a linker (e.g., to indirectly couple a moiety to the substrate). In one embodiment, a linker has one terminal functional group, a spacer region and a tumor-associated protein adhering region. The terminal functional groups for reacting with functional groups on an activated coating include halogen, amino, hydroxyl, or thiol groups. In some instances, a terminal functional group is selected from the group consisting of a carboxylic acid, halogen, amine, thiol, alkene, acrylate, anhydride, ester, acid halide, isocyanate, hydrazine, maleimide and hydroxyl group. The spacer region may include, but is not limited to, polyethers, polypeptides, polyamides, polyamines, polyesters, polysaccharides, polyols, multiple charged species or any other combinations thereof. Exemplary spacer regions include polymers of ethylene glycols, peptides, glycerol, ethanolamine, serine, inositol, etc. The spacer region may be hydrophilic in nature. The spacer region may be hydrophobic in nature. In some instances, the spacer has n oxyethylene groups, where n is between 2 and 25. In some instances, a region of a linker that adheres to a tumor-associated protein, or other moiety is hydrophobic or amphiphilic with straight or branched chain alkyl, alkynyl, alkenyl, aryl, arylalkyl, heteroalkyl, heteroalkynyl, heteroalkenyl, heteroaryl, or heteroarylalkyl. In some instances, a region of a linker that adheres to a tumor-associated protein, or other moiety comprises a C₁₀-C₂₅ straight or branched chain alkyl or heteroalkyl hydrophobic tail. In some instances, a linker comprises a terminal functional group on one end, a spacer, a tumor-associated protein adhering region, and a hydrophilic group on another end. The hydrophilic group at one end of the linker may be a single group or a straight or branched chain of multiple hydrophilic groups (e.g., a single hydroxyl group or a chain of multiple ethylene glycol units).

In some embodiments, the support can be planar. In some instances, the support can be spherical. In some instances, the support can be a bead. In some instances, a support can be magnetic. In some instances, a magnetic solid support can comprises magnetite, maghemitite, FePt, SrFe, iron, cobalt, nickel, chromium dioxide, ferrites, or mixtures thereof. In some instances, a support can be nonmagnetic. In some embodiments, the nonmagnetic solid support can comprise a polymer, metal, glass, alloy, mineral, or mixture thereof. In some instances a nonmagnetic material can be a coating around a magnetic solid support. In some instances, a magnetic material may be distributed in the continuous phase of a magnetic material. In some embodiments, the solid support comprises magnetic and nonmagnetic materials. In some instances, a solid support can comprise a combination of a magnetic material and a nonmagnetic material. In some embodiments, the magnetic material is at least about 5, 10, 20, 30, 40, 50, 60, 70, or about 80% by weight of the total composition of the solid support. In some embodiments, the bead size can be quite large, on the order of 100-900 microns or in some cases even up to a diameter of 3 mm. In other embodiments, the bead size can be on the order of 1-150 microns. The average particle diameters of beads of the invention can be in the range of about 2 μm to several millimeters, e.g., diameters in ranges having lower limits of 2 μm, 4 μm, 6 μm, 8 μm, 10 μm, 20 μm, 30 μm, 40 μm, 50 μm, 60 μm, 70 μm, 80 μm, 90 μm, 100 μm, 150 μm, 200 μm, 300 μm, or 500 μm, and upper limits of 20 μm, 30 μm, 40 μm, 50 μm, 60 μm, 70 μm, 80 μm, 90 μm, 100 μm, 150 μm, 200 μm, 300 μm, 500 μm, 750 μm, 1 mm, 2 mm, or 3 mm.

A support or substrate can be an array. In some embodiment a solid support comprises an array. An array of the invention can comprise an ordered spatial arrangement of two or more discrete regions. Address, spot, microspot, and discrete region are terms used interchangeably and refer to a particular position, such as on an array. An array can comprise tumor-associated proteins located at known or unknown discrete regions. An array can comprise tumor-associated proteins or antibodies located at known or unknown discrete regions.

Each of two or more discrete regions can comprise a tumor-associated protein. Each of two or more discrete regions can comprise a tumor-associated protein. The two or more discrete regions of an array can comprise two or more first discrete locations and two or more second discrete locations.

For example, an array can comprise a first discrete region comprising a first tumor-associated protein, and a second discrete region comprising a second tumor-associated protein.

Row and column arrangements of arrays can be selected due to the relative simplicity in making such arrangements. The spatial arrangement can, however, be essentially any form selected by the user, and optionally, in a pattern. Microspots of an array may be any convenient shape, including circular, ellipsoid, oval, annular, or some other analogously curved shape, where the shape may, in certain embodiments, be a result of the particular method employed to produce the array. The microspots may be arranged in any convenient pattern across or over the surface of the array, such as in rows and columns so as to form a grid, in a circular pattern, and the like, where generally the pattern of spots will be present in the form of a grid across the surface of the substrate.

An array can comprise an ordered spatial arrangement of two or more tumor-associated proteins, on a solid surface. For example, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 tumor-associated proteins. An array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 antibodies specific for a tumor-associated protein. The tumor-associated proteins can be linked to the array by the antibodies. Thus, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 tumor-associated proteins linked to the array by at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 antibodies specific for the tumor-associated proteins.

An array can comprise an ordered spatial arrangement of two or more same or different tumor-associated proteins, on a solid surface. For example, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 same or different tumor-associated proteins. For example, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 same or different tumor-associated proteins.

An array can be a high-density array. A high-density array can comprise tens, hundreds, thousands, tens-of-thousands or hundreds-of-thousands of tumor-associated proteins. The density of microspots of an array may be at least about 1/cm² or at least about 10/cm², up to about 1,000/cm² or up to about 500/cm². In certain embodiments, the density of all the microspots on the surface of the substrate may be up to about 400/cm², up to about 300/cm², up to about 200/cm², up to about 100/cm², up to about 90/cm², up to about 80/cm², up to about 70/cm², up to about 60/cm², or up to about 50/cm². For example, an array can comprise at least 50, 60, 70, 80, 90, 100, 150, 200, 250, 300, 350, 400, 450, 500, 600, 700, 800, 900, or 1,000 distinct antibodies per a surface area of less than about 1 cm². For example, an array can comprise 50, 60, 70, 80, 90, 100, 150, 200, 250, 300, 350 or 400 discrete regions in an area of about 16 mm², or 2,500 discrete regions/cm². In some embodiments, tumor-associated proteins, linkers, or another moiety in each discrete region are present in a defined amount (e.g., between about 0.1 femtomoles and 100 nanomoles). For example, an array can comprise at least about 2 tumor-associated proteins per cm². For example, an array can comprise at least about 2, 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, or more tumor-associated proteins. For example, an array can be a high-density protein array comprising at least about 10 tumor-associated proteins per cm². For example, an array can comprise at least about 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, or more tumor-associated proteins per cm²

Kits

Also provided are kits that find use in practicing the subject methods, as mentioned above. A kit can include one or more of the compositions described herein. A kit can include at least one tumor-associated protein. A kit can include at least one antibody. A kit can comprise barcoded tumor-associated proteins. In some embodiments, a kit can include a tumor associated protein disclosed herein

A kit can include a solid support. In some embodiments, the solid support is already functionalized with at least one tumor-associated protein. In some embodiments, the solid support is not functionalized with at least one tumor-associated protein. A kit can include a reagent for coupling at least one tumor-associated protein to the solid support.

A kit can include one or more reagents for performing amplification, including suitable primers, enzymes, nucleobases, and other reagents such as PCR amplification reagents (e.g., nucleotides, buffers, cations, etc.), and the like. Additional reagents that are required or desired in the protocol to be practiced with the kit components may be present. Such additional reagents include, but are not limited to, one or more of the following an enzyme or combination of enzymes such as a polymerase, reverse transcriptase, nickase, restriction endonuclease, uracil-DNA glycosylase enzyme, enzyme that methylates or demethylates DNA, endonuclease, ligase, etc.

As indicated above, certain protocols will employ two or more different sets of such probes for simultaneous detection of two or more tumor-associated proteins in a sample (e.g., in multiplex and/or high throughput formats). In some embodiments a kit includes two or more distinct sets of antibodies, and/or tumor-associated proteins.

The kit components may be present in separate containers, or one or more of the components may be present in the same container, where the containers may be storage containers and/or containers that are employed during the assay for which the kit is designed.

In addition to the above components, the subject kits may further include instructions for practicing the subject methods. These instructions may be present in the subject kits in a variety of forms, such as printed information on a suitable medium or substrate (e.g., a piece or pieces of paper on which the information is printed), in the packaging of the kit, in a package insert, etc. Yet another means would be a computer readable medium (e.g., diskette, CD, etc.), on which the information has been recorded. Yet another means that may be present is a website address which may be used via the internet to access the information at a removed site.

Communicating a Result

Additional embodiments of the invention can relate to the communication of assay results or diagnoses or both to technicians, physicians or subjects, for example. In certain embodiments, computers will be used to communicate results of the assessing or diagnoses or both to interested parties, e.g., physicians and their subjects. In some embodiments, the assessing can be performed or results analyzed in a country or jurisdiction which differs from the country or jurisdiction to which the results or diagnoses are communicated. In some embodiments of the invention, a diagnosis based on the presence or absence in a test subject of any biomarker identified by the invention may be communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test maybe generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions

### Other Embodiments

The section headings used herein are for organizational purposes only and are not to be construed as limiting the subject matter described.

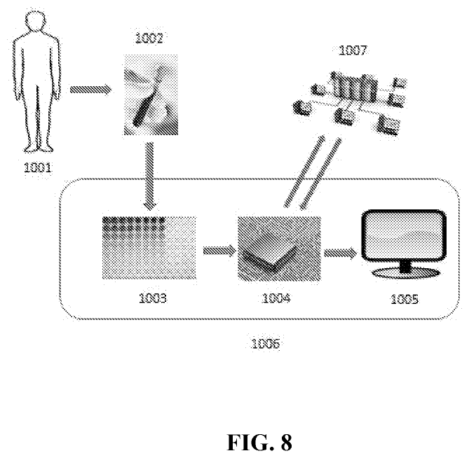

FIG. 8 shows an exemplary method for assessing LC or a disease or condition described herein in a subject. Peripheral blood (1002) can be drawn from a subject (1001). The expression of a group of biomarkers (for example autoantibodies) in the blood can be measured by an assay (1003). In some cases, the assay can be a protein-based assay, such as enzyme-linked immunosorbent assay (ELISA). In some cases, an autoantibody expression can be measured for assessing LC or a disease or condition described herein. The expression levels or signal intensity autoantibodies can be analyzed by a computer system (1004). In some cases, the computer system can compare the expression or signal of the autoantibodies to a reference. The reference can be stored in the computer system. Alternatively, the reference can be stored in other computers, databases, and/or servers, and can be accessed through a network (e.g. the Internet) (1007). The result of whether a subject has LC, a disease or condition described herein can be transmitted to an output device, e.g., a monitor (1005). The assay, the computer system, and the output device (1003, 1004, an 1005) can be integrated into a single device (10006). In some cases, such a device can be a point of care device, e.g., a portable point of care device. In some cases, the computer system can be a smartphone.

It is to be understood that the methods and compositions described herein are not limited to the particular methodology, protocols, constructs, and reagents described herein and as such may vary. It is also to be understood that the terminology used herein is for the purpose of describing particular embodiments only, and is not intended to limit the scope of the methods and compositions described herein, which will be limited only by the appended claims. While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EXAMPLES

### Example 1. Design and Summary of New Study: Identification of IgA-Autoantigens

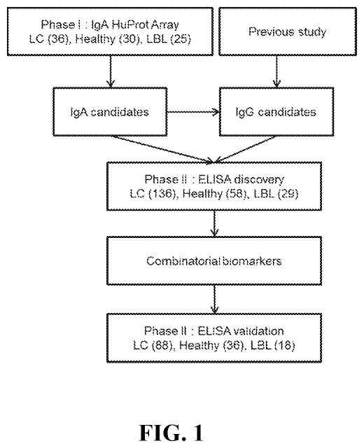

To identify IgA-autoantigens as diagnostic biomarkers for early stage LC, a two-phase strategy to identify novel biomarkers for early LC (e.g., stages 1 and 2) diagnosis was used (FIG. 1). Briefly, in Phase I, 124 serum samples collected from 69 early LC patients, 30 healthy individuals and 25 LBL patients, were individually profiled on HuProt arrays for the presence of IgA-bound autoantigens (Tables 1 and 2). A total of 28 human proteins were identified as candidate autoantigens (Table 3). In Phase II, a much larger cohort, comprised of serum samples collected from 136 early stage LC, 58 healthy subjects and 29 LBL (Tables 8 and 9), was assembled and tested against the candidate IgA autoantigens and some previously identified IgG autoantigens using ELISA. A combinatorial IgA/IgG autoantigen panel was identified with much improved performance and was further validated using an independent cohort (Table 9).

### Example 2. Collection of Serum Samples for IgA Study

All serum samples involved in this study were collected at Fujian Provincial Hospital, in Fujian Province, China, between 2015 and 2017. This cohort was comprised of 489 serum samples collected from 124 healthy persons, 293 resident patients diagnosed with early stage LC, and 72 resident patients diagnosed with lung benign lesions (LBL). The 124 healthy persons were recruited during annual physical examinations, including chest X-ray, abdominal ultrasonography, routine urinalysis, stool occult blood test, complete blood count, blood chemistries, and tumor antigen tests, such as carcinoembryonic antigen (CEA), CA199, and alphafetoprotein (AFP), to name a few. None of them showed any evidence of malignancy in all of the tests. The 293 early stage LC patients were recruited after histopathological confirmation of LC tumors. The TNM classification was used for evaluation of NSCLC staging and the VA scheme was used to classify SCLC into limited- and extensive-stages. The 72 LBL patients, including 31 pneumonia, 16 chronic obstructive pulmonary disease (COPD), and 25 pulmonary tuberculosis (TB), were recruited after accurate clinical assessment. Detailed information of each subject of this cohort is listed in Table 1. This study was approved by the Ethics Committee (i.e., IRB) of Fujian Provincial Hospital.

TABLE 1
Characteristics of serum samples for IgA study

| | | | | | | <20 | |
|---|---|---|---|---|---|---|---|
| | | | | | | pack | |
| # | Age | Sex | Type | Stage | Smoker | years | Cohort |
| 1 | 45 | Male | Small Cell Lung Cancer | Limited | | | HuProt Array |
| 2 | 51 | Male | Small Cell Lung Cancer | Limited | ✓ | ✓ | HuProt Array |
| 3 | 51 | Male | Small Cell Lung Cancer | Limited | ✓ | | HuProt Array |
| 4 | 52 | Male | Small Cell Lung Cancer | Limited | | | HuProt Array |
| 5 | 52 | Male | Small Cell Lung Cancer | Limited | ✓ | | HuProt Array |
| 6 | 54 | Male | Small Cell Lung Cancer | Limited | ✓ | ✓ | HuProt Array |
| 7 | 54 | Male | Small Cell Lung Cancer | Limited | ✓ | | HuProt Array |
| 8 | 56 | Male | Small Cell Lung Cancer | Limited | | | HuProt Array |
| 9 | 59 | Male | Small Cell Lung Cancer | Limited | ✓ | | HuProt Array |
| 10 | 60 | Male | Small Cell Lung Cancer | Limited | | | HuProt Array |
| 11 | 61 | Male | Small Cell Lung Cancer | Limited | ✓ | ✓ | HuProt Array |
| 12 | 61 | Male | Snail Cell Lung Cancer | Limited | ✓ | | HuProt Array |
| 13 | 62 | Male | Small Cell Lung Cancer | Limited | ✓ | | HuProt Array |
| 14 | 62 | Male | Small Cell Lung Cancer | Limited | | | HuProt Array |
| 15 | 63 | Male | Small Cell Lung Cancer | Limited | ✓ | ✓ | HuProt Array |
| 16 | 63 | Male | Small Cell Lung Cancer | Limited | ✓ | | HuProt Array |
| 17 | 64 | Female | Small Cell Lung Cancer | Limited | ✓ | ✓ | HuProt Array |
| 18 | 66 | Male | Small Cell Lung Cancer | Limited | ✓ | | HuProt Array |
| 19 | 66 | Male | Small Cell Lung Cancer | Limited | | | HuProt Array |
| 20 | 67 | Male | Small Cell Lung Cancer | Limited | | | HuProt Array |
| 21 | 68 | Female | Small Cell Lung Cancer | Limited | ✓ | ✓ | HuProt Array |
| 22 | 70 | Male | Small Cell Lung Cancer | Limited | ✓ | | HuProt Array |
| 23 | 71 | Male | Small Cell Lung Cancer | Limited | | | HuProt Array |
| 24 | 74 | Male | Small Cell Lung Cancer | Limited | ✓ | | HuProt Array |
| 25 | 51 | Male | Lung Adenocarcinoma | Ia | ✓ | | HuProt Array |
| 26 | 55 | Male | Lung Adenocarcinoma | Ia | ✓ | | HuProt Array |
| 27 | 40 | Female | Lung Adenocarcinoma | Ia | ✓ | | HuProt Array |
| 28 | 57 | Male | Lung Adenocarcinoma | Ia | ✓ | | HuProt Array |
| 29 | 62 | Female | Lung Adenocarcinoma | Ia | ✓ | | HuProt Array |
| 30 | 67 | Female | Lung Adenocarcinoma | Ia | ✓ | | HuProt Array |
| 31 | 65 | Male | Lung Adenocarcinoma | Ia | | | HuProt Array |
| 32 | 67 | Male | Lung Adenocarcinoma | IIa | ✓ | | HuProt Array |
| 33 | 75 | Female | Lung Adenocarcinoma | IIa | ✓ | | HuProt Array |
| 34 | 59 | Female | Lung Adenocarcinoma | IIb | | | HuProt Array |
| 35 | 61 | Female | Lung Adenocarcinoma | IIa | ✓ | ✓ | HuProt Array |
| 36 | 74 | Male | Lung Adenocarcinoma | IIa | ✓ | | HuProt Array |
| 37 | 71 | Male | Lung Adenocarcinoma | Ia | ✓ | | HuProt Array |
| 38 | 54 | Male | Lung Adenocarcinoma | Ia | ✓ | | HuProt Array |
| 39 | 66 | Male | Lung Adenocarcinoma | Ia | | | HuProt Array |
| 40 | 57 | Female | Lung Adenocarcinoma | Ib | ✓ | | HuProt Array |
| 41 | 74 | Female | Lung Adenocarcinoma | Ib | ✓ | | HuProt Array |
| 42 | 70 | Female | Lung Adenocarcinoma | Ia | ✓ | ✓ | HuProt Array |
| 43 | 52 | Male | Lung Adenocarcinoma | IIa | ✓ | | HuProt Array |
| 44 | 70 | Female | Lung Adenocarcinoma | Ia | ✓ | | HuProt Array |
| 45 | 45 | Female | Lung Adenocarcinoma | Ib | | | HuProt Array |
| 46 | 49 | Female | Lung Adenocarcinoma | Ib | ✓ | | HuProt Array |
| 47 | 44 | Female | Lung Adenocarcinoma | Ia | | | HuProt Array |
| 48 | 68 | Male | Lung Adenocarcinoma | IIa | ✓ | ✓ | HuProt Array |
| 49 | 60 | Male | Lung Adenocarcinoma | IIb | | | HuProt Array |
| 50 | 59 | Male | Lung Adenocarcinoma | Ia | | | HuProt Array |
| 51 | 77 | Male | Lung Adenocarcinoma | IIa | ✓ | ✓ | HuProt Array |
| 52 | 72 | Male | Squamous Cell Lung Cancer | IIb | ✓ | | HuProt Array |
| 53 | 63 | Male | Squamous Cell Lung Cancer | Ia | ✓ | | HuProt Array |
| 54 | 45 | Male | Squamous Cell Lung Cancer | Ia | | | HuProt Array |
| 55 | 78 | Male | Squamous Cell Lung Cancer | Ib | ✓ | | HuProt Array |
| 56 | 69 | Male | Squamous Cell Lung Cancer | Ia | | | HuProt Array |
| 57 | 71 | Male | Squamous Cell Lung Cancer | Ia | ✓ | | HuProt Array |
| 58 | 64 | Male | Squamous Cell Lung Cancer | IIb | ✓ | | HuProt Array |
| 59 | 53 | Male | Squamous Cell Lung Cancer | IIb | ✓ | | HuProt Array |
| 60 | 56 | Male | Squamous Cell Lung Cancer | Ib | | | HuProt Array |
| 61 | 64 | Male | Squamous Cell Lung Cancer | Ib | ✓ | | HuProt Array |
| 62 | 59 | Male | Squamous Cell Lung Cancer | IIb | ✓ | | HuProt Array |
| 63 | 71 | Male | Squamous Cell Lung Cancer | Ia | | | HuProt Array |
| 64 | 55 | Male | Squamous Cell Lung Cancer | Ia | ✓ | ✓ | HuProt Array |
| 65 | 57 | Male | Squamous Cell Lung Cancer | Ia | ✓ | | HuProt Array |
| 66 | 66 | Male | Squamous Cell Lung Cancer | Ia | | | HuProt Array |
| 67 | 46 | Male | Squamous Cell Lung Cancer | Ia | ✓ | | HuProt Array |
| 68 | 63 | Male | Squamous Cell Lung Cancer | Ia | ✓ | | HuProt Array |
| 69 | 67 | Male | Squamous Cell Lung Cancer | Ia | | | HuProt Array |
| 70 | 42 | Female | Healthy | | | | HuProt Array |
| 71 | 46 | Male | Healthy | | ✓ | ✓ | HuProt Array |
| 72 | 49 | Male | Healthy | | ✓ | | HuProt Array |
| 73 | 51 | Female | Healthy | | | | HuProt Array |
| 74 | 51 | Male | Healthy | | ✓ | | HuProt Array |
| 75 | 52 | Male | Healthy | | ✓ | ✓ | HuProt Array |
| 76 | 53 | Female | Healthy | | | | HuProt Array |
| 77 | 54 | Male | Healthy | | | | HuProt Array |
| 78 | 54 | Female | Healthy | | | | HuProt Array |
| 79 | 55 | Male | Healthy | | ✓ | | HuProt Array |
| 80 | 55 | Male | Healthy | | ✓ | | HuProt Array |
| 81 | 56 | Male | Healthy | | ✓ | ✓ | HuProt Array |
| 82 | 57 | Male | Healthy | | ✓ | | HuProt Array |
| 83 | 57 | Male | Healthy | | | | HuProt Array |
| 84 | 58 | Male | Healthy | | ✓ | | HuProt Array |
| 85 | 60 | Male | Healthy | | ✓ | | HuProt Array |
| 86 | 60 | Male | Healthy | | | | HuProt Array |
| 87 | 61 | Male | Healthy | | ✓ | ✓ | HuProt Anay |
| 88 | 62 | Male | Healthy | | ✓ | | HuProt Array |
| 89 | 63 | Male | Healthy | | ✓ | | HuProt Array |
| 90 | 63 | Female | Healthy | | | | HuProt Array |
| 91 | 64 | Male | Healthy | | | | HuProt Array |
| 92 | 65 | Female | Healthy | | ✓ | ✓ | HuProt Array |
| 93 | 65 | Male | Healthy | | ✓ | | HuProt Array |
| 94 | 66 | Male | Healthy | | | | HuProt Array |
| 95 | 69 | Male | Healthy | | | | HuProt Array |
| 96 | 69 | Female | Healthy | | | | HuProt Array |
| 97 | 70 | Female | Healthy | | ✓ | | HuProt Array |
| 98 | 70 | Male | Healthy | | ✓ | | HuProt Array |
| 99 | 72 | Male | Healthy | | ✓ | ✓ | HuProt Array |
| 100 | 45 | Female | Pneumonia | | ✓ | | HuProt Array |
| 101 | 48 | Female | Pneumonia | | ✓ | | HuProt Array |
| 102 | 50 | Male | Pneumonia | | | | HuProt Array |
| 108 | 60 | Female | Pneumonia | | ✓ | ✓ | HuProt Array |
| 109 | 61 | Male | Pneumonia | | | | HuProt Array |
| 110 | 61 | Male | Pneumonia | | ✓ | | HuProt Array |
| 112 | 62 | Male | Pneumonia | | | | HuProt Array |
| 115 | 64 | Male | Pneumonia | | ✓ | | HuProt Array |
| 117 | 66 | Male | Pneumonia | | ✓ | | HuProt Array |
| 118 | 66 | Male | Pneumonia | | | | HuProt Array |
| 119 | 67 | Male | Pulmonary tuberculosis | | ✓ | | HuProt Array |
| 121 | 69 | Male | Pulmonary tuberculosis | | ✓ | ✓ | HuProt Array |
| 123 | 72 | Male | Pulmonary tuberculosis | | ✓ | | HuProt Array |
| 104 | 54 | Male | Pulmonary tuberculosis | | ✓ | | HuProt Array |
| 105 | 57 | Male | Pulmonary tuberculosis | | ✓ | | HuProt Array |
| 106 | 57 | Male | Pulmonary tuberculosis | | | | HuProt Array |
| 107 | 58 | Male | Pulmonary tuberculosis | | ✓ | | HuProt Array |
| 111 | 62 | Male | Pulmonary tuberculosis | | | | HuProt Array |
| 113 | 62 | Female | Pulmonary tuberculosis | | ✓ | ✓ | HuProt Array |
| 124 | 73 | Male | Chronic obstructive pulmonary disease | | | | HuProt Array |
| 103 | 52 | Female | Chronic obstructive pulmonary disease | | ✓ | | HuProt Array |
| 114 | 62 | Male | Chronic obstructive pulmonary disease | | ✓ | | HuProt Array |
| 116 | 65 | Male | Chronic obstructive pulmonary disease | | | | HuProt Array |
| 120 | 68 | Male | Chronic obstructive pulmonary disease | | ✓ | | HuProt Array |
| 122 | 71 | Female | Chronic obstructive pulmonary disease | | ✓ | | HuProt Array |
| 125 | 44 | Female | Lung Adenocarcinoma | Ia | ✓ | ✓ | ELISA I |
| 126 | 44 | Female | Lung Adenocarcinoma | Ia | | | ELISA I |
| 127 | 55 | Male | Lung Adenocarcinoma | Ia | ✓ | | ELISA I |
| 128 | 64 | Female | Lung Adenocarcinoma | Ia | ✓ | | ELISA I |
| 129 | 50 | Male | Lung Adenocarcinoma | Ia | | | ELISA I |
| 130 | 34 | Male | Lung Adenocarcinoma | Ia | ✓ | | ELISA I |
| 131 | 69 | Male | Lung Adenocarcinoma | Ia | ✓ | ✓ | ELISA I |
| 132 | 44 | Female | Lung Adenocarcinoma | Ia | | | ELISA I |
| 133 | 71 | Male | Lung Adenocarcinoma | Ia | ✓ | | ELISA I |
| 134 | 75 | Female | Lung Adenocarcinoma | Ia | ✓ | | ELISA I |
| 135 | 64 | Female | Lung Adenocarcinoma | Ia | ✓ | | ELISA I |
| 136 | 67 | Male | Lung Adenocarcinoma | Ia | ✓ | ✓ | ELISA I |
| 137 | 60 | Female | Lung Adenocarcinoma | Ia | ✓ | | ELISA I |
| 138 | 68 | Female | Lung Adenocarcinoma | Ia | ✓ | | ELISA I |
| 139 | 41 | Female | Lung Adenocarcinoma | Ia | ✓ | ✓ | ELISA I |
| 140 | 65 | Male | Lung Adenocarcinoma | Ia | | | ELISA I |
| 141 | 65 | Female | Lung Adenocarcinoma | Ia | | | ELISA I |
| 142 | 59 | Male | Lung Adenocarcinoma | Ia | ✓ | | ELISA I |
| 143 | 24 | Male | Lung Adenocarcinoma | Ia | | | ELISA I |
| 144 | 43 | Female | Lung Adenocarcinoma | Ia | ✓ | ✓ | ELISA I |
| 145 | 69 | Female | Lung Adenocarcinoma | Ia | ✓ | | ELISA I |
| 146 | 51 | Female | Lung Adenocarcinoma | Ia | | | ELISA I |
| 147 | 59 | Male | Lung Adenocarcinoma | Ia | ✓ | | ELISA I |
| 148 | 37 | Female | Lung Adenocarcinoma | Ia | | | ELISA I |
| 149 | 63 | Male | Lung Adenocarcinoma | Ia | ✓ | | ELISA I |
| 150 | 68 | Male | Lung Adenocarcinoma | Ia | | | ELISA I |
| 151 | 70 | Male | Lung Adenocarcinoma | Ia | ✓ | | ELISA I |
| 152 | 72 | Male | Lung Adenocarcinoma | Ia | | | ELISA I |
| 153 | 63 | Male | Lung Adenocarcinoma | Ia | ✓ | ✓ | ELISA I |
| 154 | 51 | Male | Lung Adenocarcinoma | Ia | ✓ | | ELISA I |
| 155 | 57 | Female | Lung Adenocarcinoma | Ia | | | ELISA I |
| 156 | 55 | Female | Lung Adenocarcinoma | Ia | | | ELISA I |
| 157 | 45 | Female | Lung Adenocarcinoma | Ia | ✓ | | ELISA I |
| 158 | 67 | Female | Lung Adenocarcinoma | Ia | ✓ | | ELISA I |
| 159 | 51 | Female | Lung Adenocarcinoma | Ia | | | ELISA I |
| 160 | 71 | Male | Lung Adenocarcinoma | Ia | ✓ | | ELISA I |
| 161 | 68 | Female | Lung Adenocarcinoma | Ia | | | ELISA I |
| 162 | 39 | Male | Lung Adenocarcinoma | Ia | ✓ | | ELISA I |
| 163 | 39 | Male | Lung Adenocarcinoma | Ia | ✓ | | ELISA I |
| 164 | 60 | Female | Lung Adenocarcinoma | Ia | | | ELISA I |
| 165 | 77 | Female | Lung Adenocarcinoma | Ia | | | ELISA I |
| 166 | 52 | Male | Lung Adenocarcinoma | Ia | ✓ | | ELISA I |
| 167 | 57 | Female | Lung Adenocarcinoma | Ia | | | ELISA I |
| 168 | 52 | Female | Lung Adenocarcinoma | Ia | ✓ | | ELISA I |
| 169 | 57 | Male | Lung Adenocarcinoma | Ia | ✓ | | ELISA I |
| 170 | 41 | Male | Lung Adenocarcinoma | Ia | ✓ | | ELISA I |
| 171 | 54 | Female | Lung Adenocarcinoma | Ia | ✓ | | ELISA I |
| 172 | 60 | Female | Lung Adenocarcinoma | Ia | | | ELISA I |
| 173 | 58 | Male | Lung Adenocarcinoma | Ia | ✓ | | ELISA I |
| 174 | 71 | Female | Lung Adenocarcinoma | Ia | ✓ | | ELISA I |
| 175 | 70 | Female | Lung Adenocarcinoma | Ia | | | ELISA I |
| 176 | 62 | Female | Lung Adenocarcinoma | Ia | ✓ | | ELISA I |
| 177 | 60 | Male | Lung Adenocarcinoma | Ia | ✓ | ✓ | ELISA I |
| 178 | 74 | Male | Lung Adenocarcinoma | Ia | | | ELISA I |
| 179 | 64 | Male | Lung Adenocarcinoma | Ia | ✓ | | ELISA I |
| 180 | 63 | Male | Lung Adenocarcinoma | Ia | ✓ | | ELISA I |
| 181 | 55 | Female | Lung Adenocarcinoma | Ia | ✓ | | ELISA I |
| 182 | 51 | Male | Lung Adenocarcinoma | Ia | ✓ | ✓ | ELISA I |
| 183 | 40 | Female | Lung Adenocarcinoma | Ia | ✓ | ✓ | ELISA I |
| 184 | 55 | Female | Lung Adenocarcinoma | Ia | ✓ | | ELISA I |
| 185 | 71 | Male | Lung Adenocarcinoma | Ia | | | ELISA I |
| 186 | 66 | Male | Lung Adenocarcinoma | Ia | ✓ | | ELISA I |
| 187 | 70 | Female | Lung Adenocarcinoma | Ia | | | ELISA I |
| 188 | 52 | Male | Lung Adenocarcinoma | Ia | | | ELISA I |
| 189 | 74 | Male | Squamous Cell Lung Cancer | IIa | ✓ | ✓ | ELISA I |
| 190 | 46 | Male | Squamous Cell Lung Cancer | IIa | ✓ | | ELISA I |
| 191 | 71 | Male | Squamous Cell Lung Cancer | Ia | ✓ | | ELISA I |
| 192 | 63 | Male | Squamous Cell Lung Cancer | IIa | ✓ | ✓ | ELISA I |
| 193 | 67 | Male | Squamous Cell Lung Cancer | Ib | ✓ | ✓ | ELISA I |
| 194 | 64 | Male | Squamous Cell Lung Cancer | IIa | ✓ | | ELISA I |
| 195 | 71 | Male | Squamous Cell Lung Cancer | Ia | ✓ | | ELISA I |
| 196 | 56 | Male | Squamous Cell Lung Cancer | IIa | ✓ | | ELISA I |
| 197 | 62 | Male | Squamous Cell Lung Cancer | Ib | ✓ | | ELISA I |
| 198 | 47 | Male | Squamous Cell Lung Cancer | Ib | ✓ | ✓ | ELISA I |
| 199 | 59 | Male | Squamous Cell Lung Cancer | IIa | ✓ | | ELISA I |
| 200 | 45 | Male | Squamous Cell Lung Cancer | Ib | ✓ | | ELISA I |
| 201 | 70 | Male | Squamous Cell Lung Cancer | Ia | | | ELISA I |
| 202 | 51 | Male | Squamous Cell Lung Cancer | IIb | | | ELISA I |
| 203 | 70 | Male | Squamous Cell Lung Cancer | Ia | ✓ | | ELISA I |
| 204 | 63 | Male | Squamous Cell Lung Cancer | Ia | ✓ | | ELISA I |
| 205 | 77 | Male | Squamous Cell Lung Cancer | IIa | ✓ | | ELISA I |
| 206 | 64 | Male | Squamous Cell Lung Cancer | IIa | ✓ | | ELISA I |
| 207 | 45 | Male | Squamous Cell Lung Cancer | Ia | ✓ | ✓ | ELISA I |
| 208 | 72 | Male | Squamous Cell Lung Cancer | Ia | ✓ | | ELISA I |
| 209 | 72 | Male | Squamous Cell Lung Cancer | IIa | ✓ | ✓ | ELISA I |
| 210 | 57 | Male | Squamous Cell Lung Cancer | Ib | ✓ | | ELISA I |
| 211 | 60 | Male | Squamous Cell Lung Cancer | Ib | ✓ | | ELISA I |
| 212 | 65 | Male | Squamous Cell Lung Cancer | IIb | ✓ | | ELISA I |
| 213 | 77 | Male | Squamous Cell Lung Cancer | Ia | ✓ | ✓ | ELISA I |
| 214 | 76 | Male | Squamous Cell Lung Cancer | Ia | ✓ | | ELISA I |
| 215 | 50 | Male | Squamous Cell Lung Cancer | Ia | ✓ | ✓ | ELISA I |
| 216 | 59 | Female | Squamous Cell Lung Cancer | Ib | ✓ | | ELISA I |
| 217 | 68 | Male | Squamous Cell Lung Cancer | Ia | ✓ | | ELISA I |
| 218 | 48 | Female | Squamous Cell Lung Cancer | IIa | | | ELISA I |
| 219 | 59 | Male | Squamous Cell Lung Cancer | Ia | | | ELISA I |
| 220 | 72 | Male | Squamous Cell Lung Cancer | Ib | ✓ | | ELISA I |
| 221 | 69 | Male | Squamous Cell Lung Cancer | Ib | ✓ | | ELISA I |
| 222 | 57 | Male | Squamous Cell Lung Cancer | Ib | | | ELISA I |
| 223 | 65 | Male | Squamous Cell Lung Cancer | Ia | ✓ | | ELISA I |
| 224 | 54 | Male | Squamous Cell Lung Cancer | Ib | ✓ | ✓ | ELISA I |
| 225 | 58 | Male | Squamous Cell Lung Cancer | Ia | | | ELISA I |
| 226 | 58 | Male | Squamous Cell Lung Cancer | IIa | ✓ | | ELISA I |
| 227 | 57 | Male | Squamous Cell Lung Cancer | IIa | | | ELISA I |
| 228 | 70 | Male | Squamous Cell Lung Cancer | Ia | ✓ | ✓ | ELISA I |
| 229 | 49 | Male | Squamous Cell Lung Cancer | Ia | ✓ | | ELISA I |
| 230 | 55 | Male | Squamous Cell Lung Cancer | IIa | ✓ | | ELISA I |
| 231 | 66 | Male | Squamous Cell Lung Cancer | Ib | ✓ | | ELISA I |
| 232 | 58 | Male | Squamous Cell Lung Cancer | Ib | | | ELISA I |
| 233 | 81 | Male | Squamous Cell Lung Cancer | Ib | ✓ | | ELISA I |
| 234 | 71 | Female | Squamous Cell Lung Cancer | Ib | ✓ | | ELISA I |
| 235 | 64 | Male | Squamous Cell Lung Cancer | IIa | ✓ | | ELISA I |
| 236 | 53 | Female | Squamous Cell Lung Cancer | Ib | | | ELISA I |
| 237 | 54 | Male | Small Cell Lung Cancer | Limited | ✓ | | ELISA I |
| 238 | 54 | Male | Small Cell Lung Cancer | Limited | ✓ | | ELISA I |
| 239 | 64 | Male | Small Cell Lung Cancer | Limited | ✓ | | ELISA I |
| 240 | 68 | Female | Small Cell Lung Cancer | Limited | | | ELISA I |
| 241 | 61 | Male | Small Cell Lung Cancer | Limited | ✓ | ✓ | ELISA I |
| 242 | 74 | Male | Small Cell Lung Cancer | Limited | ✓ | | ELISA I |
| 243 | 65 | Male | Small Cell Lung Cancer | Limited | ✓ | | ELISA I |
| 244 | 63 | Male | Snail Cell Lung Cancer | Limited | | | ELISA I |
| 245 | 56 | Male | Small Cell Lung Cancer | Limited | ✓ | | ELISA I |
| 246 | 62 | Male | Snail Cell Lung Cancer | Limited | ✓ | | ELISA I |
| 247 | 54 | Male | Small Cell Lung Cancer | Limited | ✓ | | ELISA I |
| 248 | 51 | Male | Small Cell Lung Cancer | Limited | ✓ | | ELISA I |
| 249 | 66 | Male | Small Cell Lung Cancer | Limited | ✓ | | ELISA I |
| 250 | 62 | Male | Small Cell Lung Cancer | Limited | ✓ | ✓ | ELISA I |
| 251 | 52 | Male | Small Cell Lung Cancer | Limited | | | ELISA I |
| 252 | 66 | Male | Small Cell Lung Cancer | Limited | ✓ | | ELISA I |
| 253 | 74 | Male | Small Cell Lung Cancer | Limited | | | ELISA I |
| 254 | 67 | Male | Small Cell Lung Cancer | Limited | ✓ | | ELISA I |
| 255 | 66 | Male | Small Cell Lung Cancer | Limited | ✓ | | ELISA I |
| 256 | 52 | Male | Small Cell Lung Cancer | Limited | ✓ | | ELISA I |
| 257 | 52 | Male | Small Cell Lung Cancer | Limited | | | ELISA I |
| 258 | 70 | Male | Small Cell Lung Cancer | Limited | ✓ | | ELISA I |
| 259 | 45 | Male | Small Cell Lung Cancer | Limited | | | ELISA I |
| 260 | 61 | Male | Small Cell Lung Cancer | Limited | ✓ | | ELISA I |
| 261 | 56 | Male | Healthy | | ✓ | ✓ | ELISA I |
| 262 | 66 | Female | Healthy | | ✓ | | ELISA I |
| 263 | 63 | Female | Healthy | | ✓ | | ELISA I |
| 264 | 73 | Male | Healthy | | ✓ | | ELISA I |
| 265 | 72 | Female | Healthy | | | | ELISA I |
| 266 | 59 | Male | Healthy | | ✓ | ✓ | ELISA I |
| 267 | 58 | Male | Healthy | | | | ELISA I |
| 268 | 69 | Male | Healthy | | ✓ | | ELISA I |
| 269 | 44 | Female | Healthy | | ✓ | ✓ | ELISA I |
| 270 | 47 | Female | Healthy | | | | ELISA I |
| 271 | 49 | Male | Healthy | | ✓ | | ELISA I |
| 272 | 70 | Male | Healthy | | ✓ | | ELISA I |
| 273 | 46 | Female | Healthy | | | | ELISA I |
| 274 | 69 | Male | Healthy | | ✓ | | ELISA I |
| 275 | 57 | Male | Healthy | | | | ELISA I |
| 276 | 46 | Male | Healthy | | ✓ | | ELISA I |
| 277 | 67 | Female | Healthy | | ✓ | ✓ | ELISA I |
| 278 | 57 | Male | Healthy | | ✓ | ✓ | ELISA I |
| 279 | 59 | Female | Healthy | | | | ELISA I |
| 280 | 62 | Female | Healthy | | | | ELISA I |
| 281 | 48 | Male | Healthy | | ✓ | ✓ | ELISA I |
| 282 | 53 | Male | Healthy | | ✓ | ✓ | ELISA I |
| 283 | 64 | Female | Healthy | | ✓ | | ELISA I |
| 284 | 65 | Male | Healthy | | ✓ | ✓ | ELISA I |
| 285 | 55 | Female | Healthy | | | | ELISA I |
| 286 | 39 | Male | Healthy | | ✓ | ✓ | ELISA I |
| 287 | 68 | Male | Healthy | | ✓ | | ELISA I |
| 288 | 51 | Male | Healthy | | ✓ | | ELISA I |
| 289 | 70 | Male | Healthy | | ✓ | | ELISA I |
| 290 | 48 | Female | Healthy | | | | ELISA I |
| 291 | 57 | Male | Healthy | | ✓ | | ELISA I |
| 292 | 71 | Female | Healthy | | ✓ | | ELISA I |
| 293 | 49 | Female | Healthy | | ✓ | ✓ | ELISA I |
| 294 | 51 | Male | Healthy | | | | ELISA I |
| 295 | 48 | Female | Healthy | | ✓ | | ELISA I |
| 296 | 51 | Male | Healthy | | ✓ | | ELISA I |
| 297 | 45 | Female | Healthy | | | | ELISA I |
| 298 | 50 | Male | Healthy | | ✓ | ✓ | ELISA I |
| 299 | 48 | Female | Healthy | | ✓ | | ELISA I |
| 300 | 43 | Male | Healthy | | ✓ | | ELISA I |
| 301 | 57 | Male | Healthy | | ✓ | | ELISA I |
| 302 | 45 | Male | Healthy | | ✓ | | ELISA I |
| 303 | 52 | Male | Healthy | | | | ELISA I |
| 304 | 48 | Male | Healthy | | ✓ | | ELISA I |
| 305 | 67 | Male | Healthy | | | | ELISA I |
| 306 | 67 | Male | Healthy | | ✓ | | ELISA I |
| 307 | 44 | Male | Healthy | | ✓ | | ELISA I |
| 308 | 64 | Male | Healthy | | ✓ | | ELISA I |
| 309 | 71 | Male | Healthy | | ✓ | | ELISA I |
| 310 | 69 | Male | Healthy | | ✓ | ✓ | ELISA I |
| 311 | 67 | Male | Healthy | | ✓ | | ELISA I |
| 312 | 52 | Male | Healthy | | | | ELISA I |
| 313 | 40 | Female | Healthy | | ✓ | ✓ | ELISA I |
| 314 | 61 | Male | Healthy | | ✓ | | ELISA I |
| 315 | 38 | Male | Healthy | | ✓ | | ELISA I |
| 316 | 52 | Male | Healthy | | | | ELISA I |
| 317 | 41 | Male | Healthy | | ✓ | ✓ | ELISA I |
| 318 | 60 | Male | Healthy | | ✓ | | ELISA I |
| 319 | 67 | Female | Pneumonia | | ✓ | | ELISA I |
| 320 | 67 | Female | Pneumonia | | | | ELISA I |
| 321 | 70 | Male | Pneumonia | | ✓ | | ELISA I |
| 322 | 51 | Male | Pneumonia | | | | ELISA I |
| 323 | 46 | Male | Pneumonia | | ✓ | | ELISA I |
| 324 | 64 | Female | Pneumonia | | ✓ | | ELISA I |
| 325 | 64 | Male | Pneumonia | | ✓ | | ELISA I |
| 326 | 54 | Female | Pneumonia | | ✓ | | ELISA I |
| 327 | 60 | Male | Pneumonia | | ✓ | | ELISA I |
| 328 | 68 | Male | Pneumonia | | | | ELISA I |
| 329 | 48 | Male | Pneumonia | | | | ELISA I |
| 330 | 55 | Male | Pneumonia | | ✓ | ✓ | ELISA I |
| 331 | 70 | Female | Pneumonia | | ✓ | | ELISA I |
| 332 | 52 | Female | Pneumonia | | ✓ | | ELISA I |
| 333 | 70 | Male | Pneumonia | | | | ELISA I |
| 334 | 74 | Female | Pneumonia | | ✓ | | ELISA I |
| 335 | 72 | Male | Pneumonia | | ✓ | | ELISA I |
| 336 | 54 | Male | Pulmonary tuberculosis | | ✓ | ✓ | ELISA I |
| 337 | 63 | Male | Pulmonary tuberculosis | | ✓ | ✓ | ELISA I |
| 338 | 48 | Female | Pulmonary tuberculosis | | | | ELISA I |
| 339 | 64 | Male | Pulmonary tuberculosis | | ✓ | | ELISA I |
| 340 | 67 | Male | Pulmonary tuberculosis | | ✓ | | ELISA I |
| 341 | 60 | Male | Chronic obstructive pulmonary disease | | | | ELISA I |
| 342 | 41 | Female | Chronic obstructive pulmonary disease | | | | ELISA I |
| 343 | 57 | Male | Chronic obstructive pulmonary disease | | ✓ | | ELISA I |
| 344 | 57 | Female | Chronic obstructive pulmonary disease | | ✓ | | ELISA I |
| 345 | 50 | Male | Chronic obstructive pulmonary disease | | ✓ | | ELISA I |
| 346 | 56 | Male | Chronic obstructive pulmonary disease | | ✓ | ✓ | ELISA I |
| 347 | 58 | Male | Chronic obstructive pulmonary disease | | ✓ | | ELISA I |
| 348 | 40 | Female | Lung Adenocarcinoma | Ia | ✓ | ✓ | ELISA II |
| 349 | 65 | Male | Lung Adenocarcinoma | Ia | ✓ | | ELISA II |
| 350 | 51 | Male | Lung Adenocarcinoma | Ia | | | ELISA II |
| 351 | 57 | Male | Lung Adenocarcinoma | Ib | ✓ | | ELISA II |
| 352 | 53 | Male | Lung Adenocarcinoma | Ib | ✓ | ✓ | ELISA II |
| 353 | 71 | Female | Lung Adenocarcinoma | Ib | | | ELISA II |
| 354 | 72 | Male | Lung Adenocarcinoma | Ib | ✓ | | ELISA II |
| 355 | 52 | Male | Lung Adenocarcinoma | Ib | ✓ | ✓ | ELISA II |
| 356 | 57 | Male | Lung Adenocarcinoma | Ib | ✓ | | ELISA II |
| 357 | 56 | Female | Lung Adenocarcinoma | Ib | ✓ | | ELISA II |
| 358 | 55 | Female | Lung Adenocarcinoma | Ia | ✓ | | ELISA II |
| 359 | 49 | Female | Lung Adenocarcinoma | Ia | | | ELISA II |
| 360 | 75 | Female | Lung Adenocarcinoma | Ia | ✓ | | ELISA II |
| 361 | 45 | Female | Lung Adenocarcinoma | Ia | ✓ | | ELISA II |
| 362 | 56 | Female | Lung Adenocarcinoma | Ia | ✓ | | ELISA II |
| 363 | 67 | Female | Lung Adenocarcinoma | Ia | ✓ | | ELISA II |
| 364 | 50 | Male | Lung Adenocarcinoma | Ia | | | ELISA II |
| 365 | 75 | Female | Lung Adenocarcinoma | Ia | ✓ | | ELISA II |
| 366 | 54 | Female | Lung Adenocarcinoma | Ib | ✓ | | ELISA II |
| 367 | 55 | Female | Lung Adenocarcinoma | Ib | | | ELISA II |
| 368 | 43 | Male | Lung Adenocarcinoma | Ia | ✓ | ✓ | ELISA II |
| 369 | 63 | Male | Lung Adenocarcinoma | Ib | ✓ | | ELISA II |
| 370 | 56 | Female | Lung Adenocarcinoma | Ia | ✓ | | ELISA II |
| 371 | 75 | Female | Lung Adenocarcinoma | Ib | ✓ | | ELISA II |
| 372 | 49 | Male | Lung Adenocarcinoma | Ib | ✓ | ✓ | ELISA II |
| 373 | 51 | Female | Lung Adenocarcinoma | Ib | | | ELISA II |
| 374 | 72 | Male | Lung Adenocarcinoma | Ia | ✓ | | ELISA II |
| 375 | 70 | Female | Lung Adenocarcinoma | Ia | | | ELISA II |
| 376 | 70 | Female | Lung Adenocarcinoma | Ib | ✓ | | ELISA II |
| 377 | 51 | Male | Lung Adenocarcinoma | Ia | | | ELISA II |
| 378 | 51 | Male | Lung Adenocarcinoma | Ia | ✓ | | ELISA II |
| 379 | 73 | Male | Lung Adenocarcinoma | Ia | ✓ | ✓ | ELISA II |
| 380 | 41 | Male | Lung Adenocarcinoma | Ia | | | ELISA II |
| 381 | 70 | Female | Lung Adenocarcinoma | Ia | ✓ | | ELISA II |
| 382 | 52 | Female | Lung Adenocarcinoma | Ia | | | ELISA II |
| 383 | 56 | Female | Lung Adenocarcinoma | Ia | | | ELISA II |
| 384 | 49 | Female | Lung Adenocarcinoma | Ia | ✓ | | ELISA II |
| 385 | 48 | Female | Lung Adenocarcinoma | Ia | | | ELISA II |
| 386 | 66 | Male | Lung Adenocarcinoma | Ia | ✓ | | ELISA II |
| 387 | 70 | Female | Lung Adenocarcinoma | Ia | | | ELISA II |
| 388 | 45 | Female | Lung Adenocarcinoma | Ia | ✓ | ✓ | ELISA II |
| 389 | 66 | Female | Lung Adenocarcinoma | Ia | | | ELISA II |
| 390 | 72 | Male | Lung Adenocarcinoma | Ia | ✓ | | ELISA II |
| 391 | 54 | Male | Lung Adenocarcinoma | Ia | | | ELISA II |
| 392 | 67 | Female | Lung Adenocarcinoma | Ia | ✓ | | ELISA II |
| 393 | 75 | Female | Lung Adenocarcinoma | Ia | ✓ | | ELISA II |
| 394 | 59 | Male | Lung Adenocarcinoma | Ia | ✓ | | ELISA II |
| 395 | 57 | Male | Lung Adenocarcinoma | Ia | ✓ | | ELISA II |
| 396 | 75 | Male | Squamous Cell Lung Cancer | Ia | | | ELISA II |
| 397 | 64 | Male | Squamous Cell Lung Cancer | Ia | ✓ | | ELISA II |
| 398 | 63 | Male | Squamous Cell Lung Cancer | Ia | ✓ | | ELISA II |
| 399 | 74 | Male | Squamous Cell Lung Cancer | Ia | | | ELISA II |
| 400 | 60 | Male | Squamous Cell Lung Cancer | IIa | ✓ | | ELISA II |
| 401 | 57 | Female | Squamous Cell Lung Cancer | IIa | | | ELISA II |
| 402 | 65 | Male | Squamous Cell Lung Cancer | IIa | ✓ | | ELISA II |
| 403 | 60 | Male | Squamous Cell Lung Cancer | IIa | ✓ | | ELISA II |
| 404 | 71 | Female | Squamous Cell Lung Cancer | IIa | | | ELISA II |
| 405 | 67 | Female | Squamous Cell Lung Cancer | IIa | ✓ | | ELISA II |
| 406 | 54 | Male | Squamous Cell Lung Cancer | Ia | ✓ | | ELISA II |
| 407 | 64 | Male | Squamous Cell Lung Cancer | Ia | ✓ | | ELISA II |
| 408 | 65 | Male | Squamous Cell Lung Cancer | Ib | ✓ | ✓ | ELISA II |
| 409 | 64 | Male | Squamous Cell Lung Cancer | Ib | ✓ | | ELISA II |
| 410 | 61 | Male | Squamous Cell Lung Cancer | Ib | ✓ | | ELISA II |
| 411 | 61 | Male | Squamous Cell Lung Cancer | Ib | | | ELISA II |
| 412 | 54 | Male | Squamous Cell Lung Cancer | IIa | ✓ | | ELISA II |
| 413 | 73 | Male | Squamous Cell Lung Cancer | Ia | ✓ | | ELISA II |
| 414 | 65 | Male | Squamous Cell Lung Cancer | Ia | | | ELISA II |
| 415 | 61 | Male | Squamous Cell Lung Cancer | IIa | ✓ | | ELISA II |
| 416 | 51 | Male | Squamous Cell Lung Cancer | IIb | ✓ | | ELISA II |
| 417 | 74 | Female | Squamous Cell Lung Cancer | IIb | | | ELISA II |
| 418 | 56 | Male | Squamous Cell Lung Cancer | Ib | ✓ | | ELISA II |
| 419 | 62 | Male | Squamous Cell Lung Cancer | Ia | ✓ | | ELISA II |
| 420 | 70 | Male | Small Cell Lung Cancer | Limited | ✓ | ✓ | ELISA II |
| 421 | 63 | Male | Small Cell Lung Cancer | Limited | ✓ | | ELISA II |
| 422 | 60 | Male | Small Cell Lung Cancer | Limited | ✓ | ✓ | ELISA II |
| 423 | 21 | Female | Small Cell Lung Cancer | Limited | | | ELISA II |
| 424 | 50 | Male | Small Cell Lung Cancer | Limited | ✓ | | ELISA II |
| 425 | 46 | Male | Small Cell Lung Cancer | Limited | ✓ | | ELISA II |
| 426 | 61 | Male | Small Cell Lung Cancer | Limited | ✓ | | ELISA II |
| 427 | 63 | Male | Small Cell Lung Cancer | Limited | | | ELISA II |
| 428 | 63 | Male | Small Cell Lung Cancer | Limited | ✓ | | ELISA II |
| 429 | 48 | Male | Small Cell Lung Cancer | Limited | ✓ | | ELISA II |
| 430 | 67 | Male | Small Cell Lung Cancer | Limited | | | ELISA II |
| 431 | 42 | Male | Small Cell Lung Cancer | Limited | ✓ | ✓ | ELISA II |
| 432 | 59 | Male | Small Cell Lung Cancer | Limited | ✓ | | ELISA II |
| 433 | 64 | Female | Small Cell Lung Cancer | Limited | | | ELISA II |
| 434 | 51 | Male | Snail Cell Lung Cancer | Limited | ✓ | ✓ | ELISA II |
| 435 | 69 | Male | Small Cell Lung Cancer | Limited | ✓ | | ELISA II |
| 436 | 64 | Male | Healthy | | | | ELISA II |
| 437 | 57 | Male | Healthy | | ✓ | | ELISA II |
| 438 | 72 | Female | Healthy | | ✓ | | ELISA II |
| 439 | 57 | Male | Healthy | | ✓ | | ELISA II |
| 440 | 48 | Female | Healthy | | | | ELISA II |
| 441 | 71 | Female | Healthy | | ✓ | | ELISA II |
| 442 | 68 | Female | Healthy | | ✓ | | ELISA II |
| 443 | 42 | Male | Healthy | | ✓ | ✓ | ELISA II |
| 444 | 61 | Male | Healthy | | ✓ | | ELISA II |
| 445 | 40 | Female | Healthy | | | | ELISA II |
| 446 | 64 | Male | Healthy | | ✓ | ✓ | ELISA II |
| 447 | 53 | Male | Healthy | | ✓ | | ELISA II |
| 448 | 48 | Female | Healthy | | | | ELISA II |
| 449 | 58 | Male | Healthy | | ✓ | ✓ | ELISA II |
| 450 | 39 | Male | Healthy | | ✓ | ✓ | ELISA II |
| 451 | 50 | Female | Healthy | | | | ELISA II |
| 452 | 62 | Female | Healthy | | | | ELISA II |
| 453 | 52 | Male | Healthy | | ✓ | | ELISA II |
| 454 | 67 | Female | Healthy | | | | ELISA II |
| 455 | 56 | Female | Healthy | | | | ELISA II |
| 456 | 57 | Male | Healthy | | ✓ | | ELISA II |
| 457 | 41 | Female | Healthy | | ✓ | | ELISA II |
| 458 | 46 | Male | Healthy | | ✓ | | ELISA II |
| 459 | 61 | Male | Healthy | | | | ELISA II |
| 460 | 48 | Female | Healthy | | ✓ | | ELISA II |
| 461 | 36 | Male | Healthy | | ✓ | ✓ | ELISA II |
| 462 | 41 | Male | Healthy | | | | ELISA II |
| 463 | 72 | Male | Healthy | | | | ELISA II |
| 464 | 67 | Male | Healthy | | ✓ | | ELISA II |
| 465 | 59 | Male | Healthy | | ✓ | | ELISA II |
| 466 | 51 | Female | Healthy | | | | ELISA II |
| 467 | 42 | Male | Healthy | | ✓ | | ELISA II |
| 468 | 50 | Female | Healthy | | ✓ | | ELISA II |
| 469 | 54 | Male | Healthy | | ✓ | | ELISA II |
| 470 | 44 | Male | Healthy | | ✓ | | ELISA II |
| 471 | 74 | Male | Healthy | | | | ELISA II |
| 472 | 46 | Male | Pneumonia | | ✓ | | ELISA II |
| 473 | 62 | Male | Pneumonia | | ✓ | | ELISA II |
| 474 | 39 | Male | Pneumonia | | ✓ | | ELISA II |
| 475 | 73 | Female | Pneumonia | | ✓ | ✓ | ELISA II |
| 476 | 67 | Male | Pulmonary tuberculosis | | ✓ | | ELISA II |
| 477 | 72 | Female | Pulmonary tuberculosis | | | | ELISA II |
| 478 | 50 | Male | Pulmonary tuberculosis | | | | ELISA II |
| 479 | 51 | Male | Pulmonary tuberculosis | | ✓ | | ELISA II |
| 480 | 55 | Male | Pulmonary tuberculosis | | ✓ | ✓ | ELISA II |
| 481 | 60 | Male | Pulmonary tuberculosis | | ✓ | | ELISA II |
| 482 | 46 | Female | Pulmonary tuberculosis | | | | ELISA II |
| 483 | 66 | Female | Pulmonary tuberculosis | | | | ELISA II |
| 484 | 53 | Male | Pulmonary tuberculosis | | ✓ | | ELISA II |
| 485 | 73 | Male | Chronic obstructive pulmonary disease | | ✓ | ✓ | ELISA II |
| 486 | 66 | Female | Chronic obstructive pulmonary disease | | | | ELISA II |
| 487 | 57 | Male | Chronic obstructive pulmonary disease | | ✓ | | ELISA II |
| 488 | 57 | Male | Chronic obstructive pulmonary disease | | ✓ | | ELISA II |
| 489 | 62 | Female | Chronic obstructive pulmonary disease | | | | ELISA II |

### Example 3. HuProt Array Serum Profiling Assay for IgA Study

In Phase I, a cohort of 124 serum samples collected from 69 LC patients was assembled, including 24 SCLC, 27 adenocarcinoma, and 18 squamous-cell carcinoma, as well as 30 healthy subjects and 25 LBL patients, for candidate biomarker identification. HuProt arrays were manufactured by CDI Laboratories, Inc. Each HuProt array v3.0 is comprised of 20,240 unique human full-length proteins, covering ˜75% of the human proteome. Statistical analysis did not show any significant differences between the LC, LBL, and healthy groups in terms of age, gender or smoking history composition (Table 2).

TABLE 2
Characteristics of samples in Phase I of IgA study

| | Early LC (n = 69) | | | Healthy (n = 30) | | | LBL (n = 25) | | | |
|---|---|---|---|---|---|---|---|---|---|---|
| Variable | No. | Mean | % | No. | Mean | % | No. | Mean | % | P |
| Age (years) | | | | | | | | | | |
| Mean | | 61.2 | | | 59 | | | 61.3 | | 0.439 |
| Standard deviation | | 8.8 | | | 7.6 | | | 7.4 | | |
| Sex | | | | | | | | | | |
| Male | 54 | | 78.3 | 22 | | 73.3 | 19 | | 76 | 0.128 |
| Female | 15 | | 21.7 | 8 | | 26.7 | 6 | | 24 | |
| Smoking history | | | | | | | | | | |
| (pack-years) | | | | | | | | | | |
| 0 | 21 | | 30.4 | 11 | | 36.7 | 8 | | 32.0 | 0.124 |
| <20 | 11 | | 15.9 | 6 | | 20 | 3 | | 12.0 | |
| ≥20 | 37 | | 53.6 | 13 | | 43.3 | 14 | | 30.0 | |
| Type | | | | | | | | | | |
| Small Cell Lung Cancer | 24 | | 34.8 | | | | | | | |
| Adenocarcinoma | 27 | | 39.1 | | | | | | | |
| Squamous Cell Carcinoma | 18 | | 26.1 | | | | | | | |

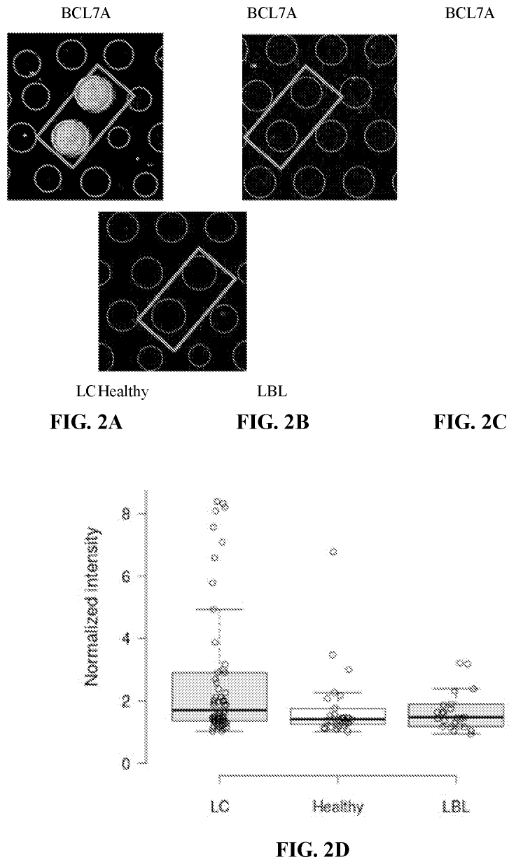

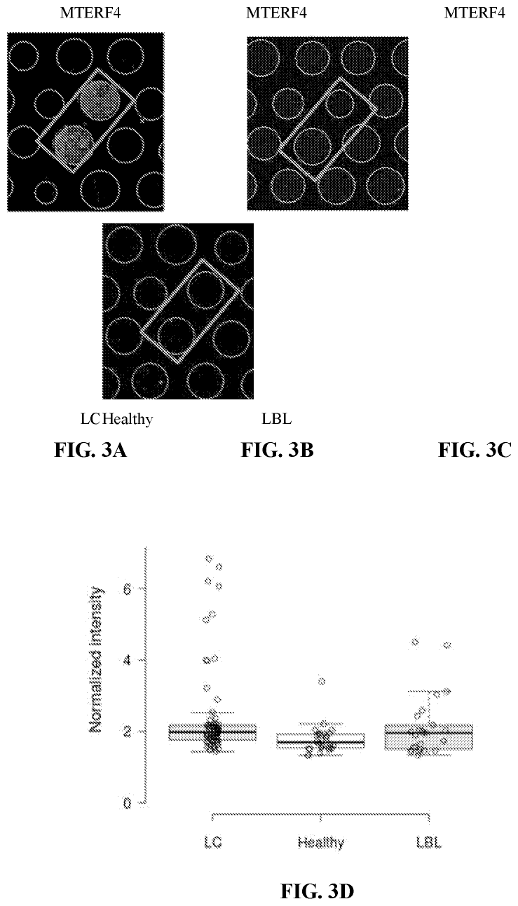

To obtain anti-human IgA autoimmune profiles, each serum sample was diluted 1000-fold and individually incubated on a HuProt array, followed by the detection of autoantigens using Cy5-labeled anti-human IgA secondary antibodies. Anti-IgA signals were acquired, normalized, and quantified for each assay, based on which standard deviation (S.D.) was calculated (Hu, S., Wan, J., Su, Y., Song, Q., Zeng, Y., Nguyen, H. N., Shin, J., Cox, E., Rho, H. S., Woodard, C., Xia, S., Liu, S., Lyu, H., Ming, G. L., Wade, H., Song, H., Qian, J., and Zhu, H. (2013) DNA methylation presents distinct binding sites for human transcription factors. Elife 2, e00726). Using a cutoff value of Z score≥3, IgA-bound target proteins were determined for each serum sample. For example, BCL7A (FIGS. 2A-2C) and MTERF4 (FIGS. 3A-3C) were preferentially recognized by human IgAs in the LC patients, but much less so in healthy subjects and LBLs. Sensitivity and specificity values were calculated for each serum-positive protein, based on which the discriminative ability values were calculated as described previously Pan, J., Song, G., Chen, D., Li, Y., Liu, S., Hu, S., Rosa, C., Eichinger, D., Pino, I., Zhu, H., Qian, J., and Huang, Y. (2017) Identification of serological biomarkers for early diagnosis of lung cancer using a protein array-based approach. Mol. Cell. Proteomics 16, 2069-2078; Riegelman, R. K. (2012) Studying A study & testing A test: Reading evidence-based health research 6th. Sixth edition ed. Wolters Kluwer/Lippincott Williams & Wilkins).

First, the median values of the foreground (Fij) and background (Bij) intensity at a given protein spot (i,j) on the HuProt arrays were extracted. The signal intensity (Rij) of each protein spot was defined as Fij/Bij. Since each protein is printed in duplicate on an array, Rij was averaged for each protein as Rp.

A Z-score of each protein on the HuProt arrays was calculated, and a cutoff value of Z≥3 was used to determine the positives in this study. The sensitivity and specificity were calculated for each protein. For each comparison (LC vs negative controls), the biomarker candidates were selected with the highest discriminant ability (Riegelman, R. K. (2012) Studying A study & testing A test: Reading evidence-based health research 6th. Sixth edition ed. Wolters Kluwer/Lippincott Williams & Wilkins), which is defined as:

$$Discriminant ability = \frac{Sensitivity + Specificity}{2}$$

P values obtained from the T test were calculated and adjusted as false discovery rates (Reich, M., Liefeld, T., Gould, J., Lerner, J., Tamayo, P., and Mesirov, J. P. (2006) GenePattern 2.0. Nat. Genet. 38, 500-501). The optimal cutoff value for each candidate was evaluated with two criteria: 1) at least 90% specificity and 2) the highest discriminant ability.

To determine which of the IgA-bound autoantigens would be subjected to Phase II confirmation using standard ELISA-format testing, candidates were first selected with specificity values>85%, which were then ranked according to their discriminative ability values. Of the 72 candidate IgA autoantigens, the top 20 candidates were selected, as well as eight additional ones that were either highly expressed in lung cancer on the basis of tissue pathology (e.g., TPM3 and TTC1) or functionally relevant in tumorigenesis (e.g., MAGEC2 and BCL7A) Performance characteristics of the 28 selected IgA biomarkers is shown in Table 3.

TABLE 3
Performance of 28 IgA biomarkers in Phase I of IgA study

| Gene | | | Discriminative | |
|---|---|---|---|---|
| symbol | Sensitivity | Specificity | ability | Rank |
| SS18 | 30.4% | 96.4% | 63.4% | 1 |
| YP021 | 27.5% | 96.4% | 61.9% | 2 |
| APH1A | 27.5% | 96.4% | 61.9% | 3 |
| TOM1L2 | 27.5% | 96.4% | 61.9% | 4 |
| PIP4K2C | 26.1% | 94.5% | 60.3% | 5 |
| NUMBL | 29.0% | 90.9% | 59.9% | 6 |
| NUDT14 | 23.2% | 96.4% | 59.8% | 7 |
| RALGDS | 24.6% | 94.5% | 59.6% | 8 |
| SPN | 18.8% | 100.0% | 59.4% | 9 |
| RPLP1 | 24.6% | 90.9% | 57.8% | 12 |
| DCLK1 | 23.2% | 90.9% | 57.0% | 15 |
| SULT2B1 | 18.8% | 94.5% | 56.7% | 17 |
| C14orf37 | 18.8% | 94.5% | 56.7% | 18 |
| MAGEA8 | 18.8% | 94.5% | 56.7% | 19 |
| DDX4 | 13.0% | 100.0% | 56.5% | 20 |
| BCL7A | 20.3% | 92.7% | 56.5% | 24 |
| NOL3 | 21.7% | 90.9% | 56.3% | 26 |
| SSBP4 | 17.4% | 94.5% | 56.0% | 28 |
| TXNDC2 | 17.4% | 94.5% | 56.0% | 29 |
| FIGNL2 | 17.4% | 94.5% | 56.0% | 30 |
| JAKMIP2 | 18.8% | 92.7% | 55.8% | 34 |
| KCNRG | 18.8% | 92.7% | 55.8% | 35 |
| N4BP1 | 21.7% | 89.1% | 55.4% | 43 |
| ACPP | 17.4% | 92.7% | 55.1% | 50 |
| TRIM33 | 17.4% | 92.7% | 55.1% | 51 |
| MTERF4 | 14.5% | 94.5% | 54.5% | 61 |
| TTC1 | 17.4% | 90.9% | 54.2% | 88 |
| MAGEC2 | 17.4% | 89.1% | 53.2% | 89 |

### Example 4. Phase II Confirmation of IgA Autoantigens as Biomarkers for Early Stage LC Diagnosis with ELISA

A new LC cohort was assembled with serum samples collected from 136 patients diagnosed with early stage LC, including 24 limited stage SCLC, 64 stage I/II adenocarcinoma, and 48 stage I/II squamous-cell carcinoma. Negative controls included 58 healthy subjects and 29 LBL patients. Statistical analysis did not find any significant differences in age, gender or smoking history between the LC group and the control groups (Table 4). All of the 28 selected candidate IgA autoantigens were successfully purified as recombinant proteins from yeast and the quantity and quality of the purified proteins were examined with Coomassie stain as previously described (Zhu, H., Bilgin, M., Bangham, R., Hall, D., Casamayor, A., Bertone, P., Lan, N., Jansen, R., Bidlingmaier, S., Houfek, T., Mitchell, T., Miller, P., Dean, R. A., Gerstein, M., and Snyder, M. (2001) Global analysis of protein activities using proteome chips. Science 293, 2101-2105).

TABLE 4
Characteristics of samples used in Phase II (discovery) of IgA study

| | Early LC (n = 136) | | | Healthy (n = 58) | | | LBL (n = 29) | | | |
|---|---|---|---|---|---|---|---|---|---|---|
| Variable | No. | Mean | % | No. | Mean | % | No. | Mean | % | P |
| Age (years) | | | | | | | | | | |
| Mean | | 59.8 | | | 56.2 | | | 59.6 | | 0.065 |
| Standard deviation | | 10.4 | | | 9.9 | | | 8.7 | | |
| Sex | | | | | | | | | | |
| Male | 98 | | 72.1 | 40 | | 69 | 19 | | 65.5 | 0.101 |
| Female | 38 | | 27.9 | 28 | | 31 | 10 | | 34.5 | |
| Smoking history | | | | | | | | | | |
| (pack-years) | | | | | | | | | | |
| 0 | 38 | | 27.9 | 15 | | 25.9 | 8 | | 27.6 | 0.371 |
| <20 | 21 | | 15.4 | 14 | | 24.1 | 4 | | 13.8 | |
| ≥20 | 77 | | 56.6 | 29 | | 50 | 17 | | 58.6 | |
| Type | | | | | | | | | | |
| Small Cell Lung Cancer | 24 | | 22.9 | | | | | | | |
| Adenocarcinoma | 64 | | 42 | | | | | | | |
| Squamous Cell Carcinoma | 48 | | 35.1 | | | | | | | |

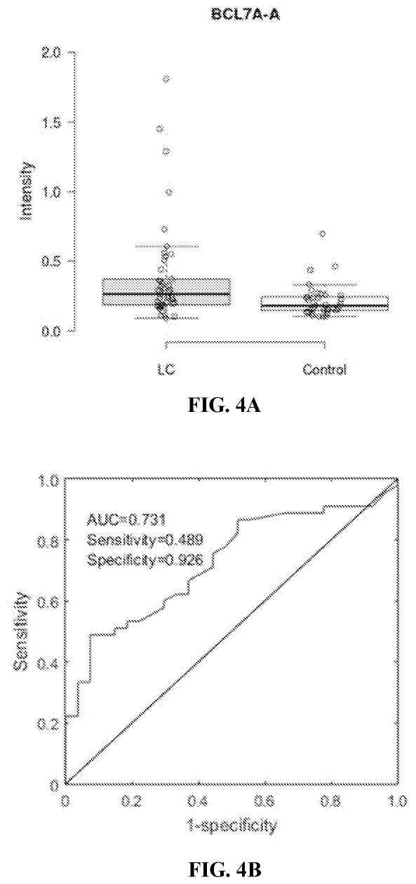

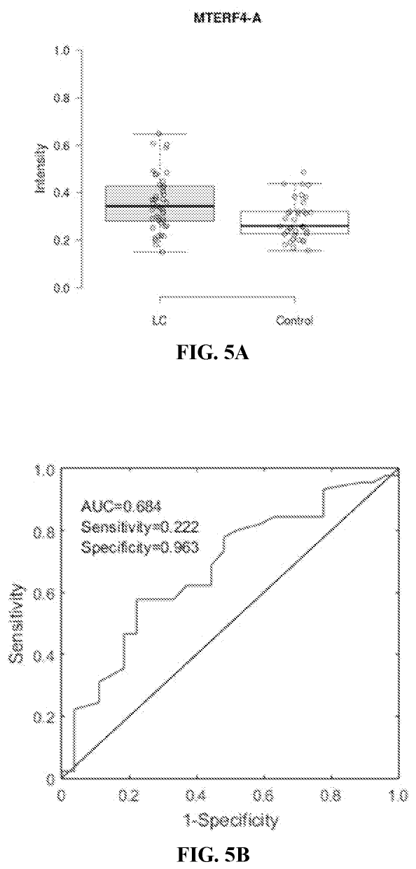

To carry out ELISA-format testing, each candidate autoantigen was coated onto individual wells of an ELISA plate, and incubated with serum samples diluted either 100-fold for IgA biomarkers or 500-fold for IgG biomarkers (Song, G., Hu, C., Zhu, H., Wang, L., Zhang, F., Li, Y., and Wu, L. (2013) New centromere autoantigens identified in systemic sclerosis using centromere protein microarrays. J. Rheumatol. 40, 461-468). After the ELISA signals were acquired and normalized to the negative controls, we performed box plot analysis, obtained the receiver operating characteristic (ROC) curves, and calculated area under the curve (AUC) values to access the performance of each candidate. For example, the box plot analysis and ROC curve for BCL7A are shown in FIGS. 4A and 4B, respectively. The box plot analysis and ROC curve for MTERF4 are shown in FIGS. 5A and 5B, respectively. The AUC values ranged from 0.503 to 0.673 for the 28 candidates, and their sensitivity values were found between 6.6% to 32.4% at a specificity value>90% (Table 5). Performance characteristics of all 28 IgA biomarkers are shown in Table 5. The analysis of the ELISA data with this new cohort confirmed the majority of the candidate biomarker proteins identified with the HuProt array approach.

TABLE 5
Performance of 28 IgA biomarkers in the
discovery stage of Phase II in IgA study

| | | | Discriminative | |
|---|---|---|---|---|
| Protein | Sensitivity | Specificity | ability | AUC |
| TRIM33 | 32.4% | 94.3% | 63.3% | 0.673 |
| BCL7A | 30.9% | 95.4% | 63.1% | 0.613 |
| ACPP | 31.6% | 93.1% | 62.4% | 0.644 |
| NUDT14 | 25.7% | 96.6% | 61.1% | 0.617 |
| DDX4 | 25.0% | 96.6% | 60.8% | 0.617 |
| MAGEA8 | 24.3% | 96.6% | 60.4% | 0.666 |
| APH1A | 24.3% | 96.6% | 60.4% | 0.544 |
| DCLK1 | 27.2% | 93.1% | 60.2% | 0.647 |
| MTERF4 | 23.5% | 96.6% | 60.0% | 0.644 |
| KCNRG | 24.3% | 95.4% | 59.8% | 0.589 |
| YP021 | 22.8% | 96.6% | 59.7% | 0.597 |
| RPLP1 | 22.1% | 96.6% | 59.3% | 0.590 |
| N4BP1 | 21.3% | 96.6% | 58.9% | 0.648 |
| MAGEC2 | 22.1% | 94.3% | 58.2% | 0.637 |
| SS18 | 19.1% | 95.4% | 57.3% | 0.528 |
| TOM1L2 | 17.6% | 96.6% | 57.1% | 0.559 |
| SSBP4 | 15.4% | 97.7% | 56.6% | 0.646 |
| C14ORF37 | 17.6% | 94.3% | 55.9% | 0.615 |
| SULT2B1 | 14.0% | 97.7% | 55.8% | 0.610 |
| TTC1 | 18.4% | 93.1% | 55.7% | 0.503 |
| JAKMIP2 | 16.9% | 94.3% | 55.6% | 0.581 |
| NUMBL | 13.2% | 97.7% | 55.5% | 0.551 |
| TXDNC2 | 15.4% | 95.4% | 55.4% | 0.601 |
| FIGNL2 | 11.0% | 98.9% | 54.9% | 0.598 |
| PIP4K2C | 14.0% | 95.4% | 54.7% | 0.512 |
| SPN | 13.2% | 95.4% | 54.3% | 0.584 |
| RALGDS | 8.8% | 97.7% | 53.3% | 0.553 |
| NOL3 | 6.6% | 95.4% | 51.0% | 0.599 |

### Example 5. Discovery and Validation of Integrated IgA/IgG Biomarker Panels

It was hypothesized that integration of IgA and IgG biomarkers would yield combined marker panels with improved performance. To test this hypothesis, the IgG seroreactivity of the same LC discovery cohort used in Example 4 was tested against the eight top IgG biomarkers described in Example 3, as well as TRIM33, MAGEC2, DDX4, RALGDS, BCL7A, SULT2B1, DCLK1 and NOL3, using the same ELISA protocol as in Example 4. Performance characteristics of the 16 biomarkers are shown in Table 6.

TABLE 6
Performance of 16 IgG biomarkers in the validation
stage of Phase II in the IgA study.

| | | | Discriminative | |
|---|---|---|---|---|
| Protein | Sensitivity | Specificity | ability | AUC |
| TP53-G | 33.1% | 93.1% | 63.1% | 0.643 |
| MAGEC2-G | 27.9% | 95.4% | 61.7% | 0.594 |
| DDX4-G | 25.0% | 96.6% | 60.8% | 0.542 |
| CTAG1A-G | 23.5% | 97.7% | 60.6% | 0.647 |
| HRAS-G | 19.1% | 97.7% | 58.4% | 0.548 |
| NSG1-G | 20.6% | 95.4% | 58.0% | 0.595 |
| TEX264-G | 21.3% | 93.1% | 57.2% | 0.602 |
| ETHE1-G | 22.1% | 92.0% | 57.0% | 0.553 |
| C1QTNF1-G | 16.2% | 97.7% | 56.9% | 0.522 |
| RALGDS-G | 18.4% | 95.4% | 56.9% | 0.566 |
| TRIM33-G | 16.9% | 95.4% | 56.2% | 0.503 |
| CLDN2-G | 15.4% | 95.4% | 55.4% | 0.548 |
| BCL7A-G | 17.6% | 93.1% | 55.4% | 0.606 |
| SULT2B1-G | 14.7% | 94.3% | 54.5% | 0.535 |
| DCLK1-G | 8.8% | 97.7% | 53.3% | 0.578 |
| NOL3-G | 6.6% | 98.9% | 52.7% | 0.485 |

To identify the optimal combination of IgA/IgG biomarker panels, the performance for all possible combinations between two and six proteins (=2,391,459 combinations) was evaluated in the validation cohort. Performance characteristics for the best performing combinations are shown in Table 7. First, the actual signal intensity of each protein was converted to either 1 or 0 such that 1 represented the signal intensity greater than the optimal cutoff value, and 0 otherwise. Next, for a given combination of n proteins, the sum of the binary scores of the n proteins was assigned to each serum sample as a summary score. If the summary score of a sample was greater than k (1≤k≤n), the sample was called positive. The sensitivity and specificity at the best discriminant ability values were recorded for each combination. Finally, the combination and its k value were identified with the best discriminant ability at a minimum specificity of 85%.

TABLE 7
Performance characteristics of panels of IgG and IgA autoantigens.

| Panel | | Cut- | Discriminative |
|---|---|---|---|
| size | Proteins | off | ability |
| 6 | MTERF4-A, MAGEC2-A,TXDNC2-A, BCL7A-G, FIGNL2-A, NOL3-A | 1 | High |
| 6 | BCL7A-A, DDX4-G, CTAG1A-G, MAGEC2-G, MTERF4-A, YP021-A | 1 | High |
| 6 | TRIM33-A, BCL7A-A, DDX4-G, CTAG1A-G, KCNRG-A, C1QTNF1-G | 1 | High |
| 6 | ACPP-A, DCLK1-A, SS18-A, TOM1L2-A, C14ORF37-A, NOL3-A | 1 | High |
| 6 | TRIM33-A, BCL7A-A, CTAG1A-G, MAGEC2-G, MTERF4-A, RALGDS-A | 1 | High |
| 6 | BCL7A-A, DDX4-G, CTAG1A-G, MAGEC2-G, RPLP1-A, N4BP1-A | 1 | High |
| 6 | BCL7A-A, DDX4-G, CTAG1A-G, MAGEC2-G, MAGEA8-A, HRAS-G | 1 | High |
| 6 | TRIM33-A, BCL7A-A, DDX4-G, CTAG1A-G, APH1A-A, C1QTNF1-G | 1 | High |
| 6 | BCL7A-A, DDX4-G, CTAG1A-G, MAGEC2-G, MTERF4-A, C1QTNF1-G | 1 | High |
| 6 | MAGEC2-G, TRIM33-G, TXDNC2-A, SULT2B1-G, SPN-A, NOL3-A | 1 | High |
| 6 | BCL7A-A, DDX4-G, CTAG1A-G, MTERF4-A, NUDT14-A, APH1A-A | 1 | High |
| 6 | MAGEC2-G, TTC1-A, JAKMIP2-A, TXDNC2-A, SULT2B1-G, NOL3-A | 1 | High |
| 6 | MAGEC2-G, TRIM33-G, TXDNC2-A, BCL7A-G, SPN-A, NOL3-A | 1 | High |
| 6 | BCL7A-A, DDX4-G, CTAG1A-G, MAGEC2-G, MTERF4-A, FIGNL2-A | 1 | High |
| 6 | BCL7A-A, DDX4-G, CTAG1A-G, MAGEC2-G, N4BP1-A, TRIM33-G | 1 | High |
| 6 | ACPP-A, RPLP1-A, TOM1L2-A, C14ORF37-A, TXDNC2-A, FIGNL2-A | 1 | High |
| 6 | BCL7A-A, DDX4-G, CTAG1A-G, MAGEC2-G, RPLP1-A, HRAS-G | 1 | High |
| 6 | BCL7A-A, DDX4-G, CTAG1A-G, MTERF4-A, KCNRG-A, SSBP4-A | 1 | High |
| 6 | BCL7A-A, DDX4-G, CTAG1A-G, MAGEC2-G, MAGEA8-A, SSBP4-A | 1 | High |
| 6 | BCL7A-A, DDX4-G, CTAG1A-G, MAGEC2-G, DDX4-A, RALGDS-A | 1 | High |
| 6 | ACPP-A, DCLK1-A, SS18-A, TOM1L2-A, SPN-A, NOL3-A | 1 | High |
| 6 | ACPP-A, DCLK1-A, TOM1L2-A, TXDNC2-A, FIGNL2-A, NOL3-A | 1 | High |
| 6 | BCL7A-A, DDX4-G, CTAG1A-G, MAGEC2-G, MTERF4-A, NUDT14-A | 1 | High |
| 6 | MAGEC2-G, TEX264-G, TTC1-A, JAKMIP2-A, TXDNC2-A, NOL3-A | 1 | High |
| 6 | ACPP-A, DCLK1-A, RPLP1-A, TOM1L2-A, C14ORF37-A, JAKMIP2-A | 1 | High |
| 6 | TRIM33-A, BCL7A-A, DDX4-G, CTAG1A-G, MAGEC2-G, MTERF4-A | 1 | High |
| 6 | BCL7A-A, DDX4-G, CTAG1A-G, MAGEC2-G, MAGEA8-A, RPLP1-A | 1 | High |
| 6 | MTERF4- A, TXDNC2-A, BCL7A-G, FIGNL2-A, RALGDS-A, NOL3-A | 1 | High |
| 6 | MAGEC2-G, C14ORF37-A, TXDNC2-A, SULT2B1-G, DCLK1-G, NOL3-A | 1 | High |
| 6 | ACPP-A, DCLK1-A, RPLP1-A, SS18-A, TOM1L2-A, FIGNL2-A | 1 | High |
| 6 | BCL7A-A, DDX4-G, CTAG1A-G, MAGEC2-G, N4BP1-A, SSBP4-A | 1 | High |
| 6 | BCL7A-A, DDX4-G, CTAG1A-G, MAGEC2-G, KCNRG-A, RPLP1-A | 1 | High |
| 6 | BCL7A-A, DDX4-G, CTAG1A-G, MTERF4-A, DDX4-A, YP021-A | 1 | High |
| 6 | MAGEC2-G, SSBP4-A, JAKMIP2-A, TXDNC2-A, SULT2B1-G, NOL3-A | 1 | High |
| 6 | BCL7A-A, DDX4-G, CTAG1A-G, YP021-A, RPLP1-A, SSBP4-A | 1 | High |
| 6 | MTERF4-A, RPLP1-A, TOM1L2-A, C14ORF37-A, TTC1-A, NOL3-A | 1 | High |
| 6 | MTERF4-A, MAGEA8-A, MAGEC2-A, TOM1L2-A, JAKMIP2-A, NOL3-A | 1 | High |
| 6 | MAGEC2-G, TEX264-G, TXDNC2-A, BCL7A-G, SPN-A, NOL3-A | 1 | High |
| 6 | BCL7A-A, DDX4-G, CTAG1A-G, MAGEC2-G, KCNRG-A, SSBP4-A | 1 | High |
| 6 | BCL7A-A, DDX4-G, CTAG1A-G, MAGEC2-G, MTERF4-A, SULT2B1-G | 1 | High |
| 6 | ACPP-A, DCLK1-A, SS18-A, TOM1L2-A, RALGDS-A, NOL3-A | 1 | High |
| 6 | BCL7A-A, DDX4-G, CTAG1A-G, KCNRG-A, RPLP1-A, SSBP4-A | 1 | High |
| 6 | MTERF4-A, MAGEA8-A, MAGEC2-A, BCL7A-G, FIGNL2-A, NOL3-A | 1 | High |
| 6 | MAGEC2-G, TEX264-G, JAKMIP2-A, TXDNC2-A, SULT2B1-G, NOL3-A | 1 | High |
| 6 | BCL7A-A, DDX4-G, CTAG1A-G, MAGEC2-G, APH1A-A, RPLP1-A | 1 | High |
| 6 | CTAG1A-G, TOM1L2-A, TXDNC2-A, SULT2B1-G, SPN-A, NOL3-A | 1 | High |
| 6 | ACPP-A, RPLP1-A, SS18-A, TOM1L2-A, C14ORF37-A, NOL3-A | 1 | High |
| 6 | TRIM33-A, BCL7A-A, CTAG1A-G, MTERF4-A, YP021-A, C1QTNF1-G | 1 | High |
| 6 | ACPP-A, RPLP1-A, TOM1L2-A, TXDNC2-A, SPN-A, NOL3-A | 1 | High |
| 6 | MAGEC2-G, TEX264-G, TRIM33-G, TXDNC2-A, SULT2B1-G, NOL3-A | 1 | High |
| 6 | BCL7A-A, DDX4-G, CTAG1A-G, APH1A-A, RPLP1-A, SSBP4-A | 1 | High |
| 6 | BCL7A-A, DDX4-G, CTAG1A-G, MTERF4-A, APH1A-A, SSBP4-A | 1 | High |
| 5 | TRIM33-A, BCL7A-A, DDX4-G, CTAG1A-G, SSBP4-A | 1 | High |
| 6 | TRIM33-A, BCL7A-A, DDX4-G, CTAG1A-G, APH1A-A, SSBP4-A | 1 | High |
| 5 | BCL7A-A, DDX4-G, CTAG1A-G, MAGEC2-G, NUDT14-A | 1 | High |
| 6 | BCL7A-A, DDX4-G, CTAG1A-G, MAGEC2-G, NUDT14-A, SSBP4-A | 1 | High |
| 6 | MAGEC2-G, TXDNC2-A, BCL7A-G, FIGNL2-A, SPN-A, NOL3-A | 1 | High |
| 6 | ACPP-A, APH1A-A, DCLK1-A, TOM1L2-A, JAKMIP2-A, NOL3-A | 1 | High |
| 5 | TRIM33-A, BCL7A-A, DDX4-G, CTAG1A-G, C1QTNF1-G | 1 | High |
| 6 | ACPP-A, NUDT14-A, DCLK1-A, RPLP1-A, SS18-A, NOL3-A | 1 | High |
| 6 | TRIM33-A, BCL7A-A, DDX4-G, CTAG1A-G, MTERF4-A, NUMBL-A | 1 | High |
| 6 | BCL7A-A, DDX4-G, CTAG1A-G, MAGEC2-G, MTERF4-A, RPLP1-A | 1 | High |
| 6 | BCL7A-A, DDX4-G, CTAG1A-G, MAGEC2-G, NUDT14-A, NOL3-G | 1 | High |
| 6 | BCL7A-A, CTAG1A-G, MAGEC2-G, MTERF4-A, DDX4-A, RALGDS-A | 1 | High |
| 6 | MAGEC2-G, TOM1L2-A, JAKMIP2-A, SULT2B1-G, DCLK1-G, NOL3-A | 1 | High |
| 6 | ACPP-A, DCLK1-A, RPLP1-A, SS18-A, JAKMIP2-A, NOL3-A | 1 | High |
| 6 | TRIM33-A, BCL7A-A, DDX4-G, CTAG1A-G, RPLP1-A, C1QTNF1-G | 1 | High |
| 5 | TRIM33-IgA, SSBP4-IgA, TP53-IgG, NSG1-IgG, CTAG1A-IgG | 1 | High |
| 5 | TRIM33-IgA, MTERF4-IgA, TP53-IgG, MAGEC2-IgG, CTAG1A-IgG | 1 | High |
| 6 | BCL7A-A, DDX4-G, CTAG1A-G, MAGEC2-G, N4BP1-A, RALGDS-A | 1 | Median |
| 6 | BCL7A-A, DDX4-G, CTAG1A-G, MAGEC2-G, RPLP1-A, SSBP4-A | 1 | Median |
| 6 | TRIM33-A, BCL7A-A, DDX4-G, CTAG1A-G, MTERF4-A, SSBP4-A | 1 | Median |
| 6 | TRIM33-A, BCL7A-A, DDX4-G, CTAG1A-G, C1QTNF1-G, FIGNL2-A | 1 | Median |
| 6 | ACPP-A, DCLK1-A, SS18-A, TOM1L2-A, JAKMIP2-A, NOL3-A | 1 | Median |
| 6 | ACPP-A, NUDT14-A, DCLK1-A, RPLP1-A, SS18-A, TOM1L2-A | 1 | Median |
| 6 | ACPP-A, NUDT14-A, DCLK1-A, RPLP1-A, TOM1L2-A, JAKMIP2-A | 1 | Median |
| 6 | TRIM33-A, BCL7A-A, DDX4-G, CTAG1A-G, MTERF4-A, RALGDS-G | 1 | Median |
| 6 | TRIM33-A, BCL7A-A, DDX4-G, CTAG1A-G, MTERF4-A, C1QTNF1-G | 1 | Median |
| 6 | ACPP-A, DCLK1-A, TOM1L2-A, FIGNL2-A, SPN-A, NOL3-A | 1 | Median |
| 6 | ACPP-A, DCLK1-A, RPLP1-A, TOM1L2-A, FIGNL2-A, NOL3-A | 1 | Median |
| 6 | BCL7A-A, DDX4-G, CTAG1A-G, MAGEC2-G, NSG1-G, NUMBL-A | 1 | Median |
| 6 | ACPP-A, DCLK1-A, RPLP1-A, SS18-A, TOM1L2-A, NOL3-A | 1 | Median |
| 6 | BCL7A-A, DDX4-G, CTAG1A-G, MAGEC2-G, MTERF4-A, RALGDS-G | 1 | Median |
| 6 | BCL7A-A, CTAG1A-G, MAGEC2-G, MTERF4-A, NUDT14-A, NUMBL-A | 1 | Median |
| 6 | ACPP-A, NUDT14-A, DCLK1-A, SS18-A, TOM1L2-A, FIGNL2-A | 1 | Median |
| 6 | MAGEC2-G, C14ORF37-A, PIP4K2C-A, SULT2B1-G, DCLK1-G, NOL3-A | 1 | Median |
| 6 | BCL7A-A, DDX4-G, CTAG1A-G, MAGEC2-G, NSG1-G, RALGDS-A | 1 | Median |
| 5 | BCL7A-A, DDX4-G, CTAG1A-G, MAGEC2-G, RPLP1-A | 1 | Median |
| 6 | ACPP-A, NUDT14-A, DCLK1-A, SS18-A, FIGNL2-A, NOL3-A | 1 | Median |
| 6 | BCL7A-A, CTAG1A-G, MAGEC2-G, MTERF4-A, SS18-A, NUMBL-A | 1 | Median |
| 6 | BCL7A-A, DDX4-G, CTAG1A-G, MAGEC2-G, MAGEA8-A, TRIM33-G | 1 | Median |
| 6 | ACPP-A, NUDT14-A, DCLK1-A, RPLP1-A, JAKMIP2-A, NOL3-A | 1 | Median |
| 6 | BCL7A-A, DDX4-G, CTAG1A-G, MAGEC2-G, MTERF4-A, SSBP4-A | 1 | Median |
| 6 | MAGEC2-G, TXDNC2-A, SULT2B1-G, SPN-A, DCLK1-G, NOL3-A | 1 | Median |
| 6 | TRIM33-A, BCL7A-A, DDX4-G, CTAG1A-G, MTERF4-A, HRAS-G | 1 | Median |
| 6 | MAGEC2-G, TOM1L2-A, TTC1-A, JAKMIP2-A, DCLK1-G, NOL3-A | 1 | Median |
| 6 | ACPP-A, RPLP1-A, SS18-A, TOM1L2-A, C14ORF37-A, FIGNL2-A | 1 | Median |
| 6 | MAGEC2-G, TXDNC2-A, FIGNL2-A, SPN-A, DCLK1-G, NOL3-A | 1 | Median |
| 6 | MAGEC2-G, TXDNC2-A, FIGNL2-A, SULT2B1-G, DCLK1-G, NOL3-A | 1 | Median |
| 6 | BCL7A-A, DDX4-G, CTAG1A-G, MAGEC2-G, NUDT14-A, FIGNL2-A | 1 | Median |
| 6 | TRIM33-A, BCL7A-A, CTAG1A-G, MAGEC2-G, MTERF4-A, TRIM33-G | 1 | Median |
| 6 | MAGEC2-G, TTC1-A, JAKMIP2-A, TXDNC2-A, DCLK1-G, NOL3-A | 1 | Median |
| 5 | TRIM33-A, BCL7A-A, CTAG1A-G, MAGEC2-G, C1QTNF1-G | 1 | Median |
| 6 | MTERF4-A, TOM1L2-A, TXDNC2-A, BCL7A-G, SULT2B1-G, NOL3-A | 1 | Median |
| 6 | MAGEC2-G, RPLP1-A, TOM1L2-A, JAKMIP2-A, DCLK1-G, NOL3-A | 1 | Median |
| 6 | BCL7A-A, DDX4-G, CTAG1A-G, MAGEC2-G, MTERF4-A, NUMBL-A | 1 | Median |
| 6 | BCL7A-A, DDX4-G, CTAG1A-G, MAGEC2-G, TOM1L2-A, SSBP4-A | 1 | Median |
| 6 | BCL7A-A, DDX4-G, CTAG1A-G, MAGEC2-G, SSBP4-A, TRIM33-G | 1 | Median |
| 6 | ACPP-A, NUDT14-A, DCLK1-A, RPLP1-A, TOM1L2-A, NOL3-A | 1 | Median |
| 6 | MTERF4-A, RPLP1-A, TOM1L2-A, TTC1-A, JAKM1P2-A, NOL3-A | 1 | Median |
| 6 | MAGEC2-G, TRIM33-G, TXDNC2-A, FIGNL2-A, SPN-A, NOL3-A | 1 | Median |
| 5 | BCL7A-A, DDX4-G, CTAG1A-G, MAGEC2-G, DDX4-A | 1 | Median |
| 6 | MTERF4-A, NSG1-G, TEX264-G, TTC1-A, JAKMIP2-A, NOL3-A | 1 | Median |
| 6 | BCL7A-A, DDX4-G, CTAG1A-G, MTERF4-A, YP021-A, SSBP4-A | 1 | Median |
| 6 | MAGEC2-G, SSBP4-A, TXDNC2-A, FIGNL2-A, SPN-A, NOL3-A | 1 | Median |
| 6 | ACPP-A, DCLK1-A, TOM1L2-A, TTC1-A, JAKMIP2-A, NOL3-A | 1 | Median |
| 6 | BCL7A-A, DDX4-G, CTAG1A-G, MAGEC2-G, NUDT14-A, RALGDS-A | 1 | Median |
| 6 | ACPP-A, RPLP1-A, TOM1L2-A, JAKMIP2-A, FIGNL2-A, NOL3-A | 1 | Median |
| 6 | MAGEC2-G, TEX264-G, TRIM33-G, TXDNC2-A, SPN-A, NOL3-A | 1 | Median |
| 6 | ACPP-A, RPLP1-A, SS18-A, TOM1L2-A, JAKMIP2-A, NOL3-A | 1 | Median |
| 6 | ACPP-A, DCLK1-A, SS18-A, TOM1L2-A, TTC1-A, NOL3-A | 1 | Median |
| 5 | BCL7A-A, DDX4-G, CTAG1A-G, MAGEC2-G, N4BP1-A | 1 | Median |
| 6 | MAGEC2-G, NSG1-G, TEX264-G, TRIM33-G, JAKMIP2-A, NOL3-A | 1 | Median |
| 6 | BCL7A-A, DDX4-G, CTAG1A-G, MAGEA8-A, RPLP1-A, SSBP4-A | 1 | Median |
| 5 | BCL7A-A, DDX4-G, CTAG1A-G, MAGEC2-G, MAGEA8-A | 1 | Median |
| 6 | ACPP-A, DCLK1-A, SS18-A, TOM1L2-A, TXDNC2-A, NOL3-A | 1 | Median |
| 6 | ACPP-A, DCLK1-A, SS18-A, FIGNL2-A, SPN-A, NOL3-A | 1 | Median |
| 6 | MAGEC2-G, TXDNC2-A, BCL7A-G, SPN-A, DCLK1-G, NOL3-A | 1 | Median |
| 6 | BCL7A-A, DDX4-G, CTAG1A-G, MTERF4-A, TP53-G, KCNRG-A | 1 | Median |
| 6 | ACPP-A, DCLK1-A, TOM1L2-A, JAKMIP2-A, FIGNL2-A, NOL3-A | 1 | Median |
| 6 | BCL7A-A, DDX4-G, CTAG1A-G, MAGEC2-G, RPLP1-A, TRIM33-G | 1 | Median |
| 6 | BCL7A-A, DDX4-G, CTAG1A-G, MAGEC2-G, MTERF4-A, NSG1-G | 1 | Median |
| 6 | ACPP-A, NUDT14-A, DCLK1-A, TOM1L2-A, TXDNC2-A, NOL3-A | 1 | Median |
| 6 | BCL7A-A, DDX4-G, CTAG1A-G, MTERF4-A, ACPP-A, C1QTNF1-G | 1 | Median |
| 5 | BCL7A-A, DDX4-G, CTAG1A-G, MAGEC2-G, MTERF4-A | 1 | Median |
| 6 | MAGEC2-G, JAKMIP2-A, TXDNC2-A, SULT2B1-G, DCLK1-G, NOL3-A | 1 | Median |
| 6 | MAGEC2-G, JAKMIP2-A, TXDNC2-A, FIGNL2-A, SULT2B1-G, NOL3-A | 1 | Low |
| 6 | ACPP-A, APH1A-A, DCLK1-A, SS18-A, TOM1L2-A, NOL3-A | 1 | Low |
| 6 | BCL7A-A, DDX4-G, CTAG1A-G, MTERF4-A, RPLP1-A, SSBP4-A | 1 | Low |
| 6 | BCL7A-A, DDX4-G, CTAG1A-G, MAGEC2-G, MTERF4-A, N4BP1-A | 1 | Low |
| 6 | BCL7A-A, DDX4-G, CTAG1A-G, MAGEC2-G, MTERF4-A, TRIM33-G | 1 | Low |
| 6 | TRIM33-A, BCL7A-A, DDX4-G, CTAG1A-G, KCNRG-A, SSBP4-A | 1 | Low |
| 6 | MAGEC2-G, TXDNC2-A, FIGNL2-A, SULT2B1-G, SPN-A, NOL3-A | 1 | Low |
| 6 | ACPP-A, DCLK1-A, RPLP1-A, TOM1L2-A, JAKMIP2-A, NOL3-A | 1 | Low |
| 6 | ACPP-A, DCLK1-A, SS18-A, TOM1L2-A, DCLK1-G, NOL3-A | 1 | Low |
| 6 | BCL7A-A, DDX4-G, CTAG1A-G, MAGEC2-G, APH1A-A, SSBP4-A | 1 | Low |
| 6 | BCL7A-A, DDX4-G, CTAG1A-G, MAGEC2-G, NUDT14-A, TRIM33-G | 1 | Low |
| 6 | BCL7A-A, DDX4-G, CTAG1A-G, MAGEC2-G, KCNRG-A, N4BP1-A | 1 | Low |
| 6 | BCL7A-A, DDX4-G, CTAG1A-G, MAGEC2-G, N4BP1-A, NOL3-G | 1 | Low |
| 6 | BCL7A-A, DDX4-G, CTAG1A-G, MAGEC2-G, YP021-A, SSBP4-A | 1 | Low |
| 6 | MAGEC2-G, NSG1-G, TEX264-G, JAKMIP2-A, SULT2B1-G, NOL3-A | 1 | Low |
| 6 | ACPP-A, NUDT14-A, SS18-A, TOM1L2-A, FIGNL2-A, NOL3-A | 1 | Low |
| 6 | MTERF4-A, RPLP1-A, SS18-A, TOM1L2-A, JAKMIP2-A, NOL3-A | 1 | Low |
| 6 | MAGEC2-G, TXDNC2-A, BCL7A-G, SULT2B1-G, DCLK1-G, NOL3-A | 1 | Low |
| 6 | ACPP-A, DCLK1-A, SS18-A, TOM1L2-A, C14ORF37-A, FIGNL2-A | 1 | Low |
| 6 | BCL7A-A, DDX4-G, CTAG1A-G, MAGEC2-G, MTERF4-A, SULT2B1-A | 1 | Low |
| 6 | BCL7A-A, DDX4-G, CTAG1A-G, MAGEC2-G, RPLP1-A, RALGDS-A | 1 | Low |
| 6 | ACPP-A, DCLK1-A, RPLP1-A, SS18-A, TOM1L2-A, JAKMIP2-A | 1 | Low |
| 6 | DDX4-G, CTAG1A-G, MTERF4-A, YP021-A, RALGDS-G, PIP4K2C-A | 1 | Low |
| 6 | MTERF4-A, TOM1L2-A, TTC1-A, JAKMIP2-A, FIGNL2-A, NOL3-A | 1 | Low |
| 6 | ACPP-A, DCLK1-A, SS18-A, TOM1L2-A, FIGNL2-A, SPN-A | 1 | Low |
| 6 | CTAG1A-G, TOM1L2-A, TXDNC2-A, SULT2B1-G, DCLK1-G, NOL3-A | 1 | Low |
| 6 | BCL7A-A, DDX4-G, CTAG1A-G, MAGEC2-G, MTERF4-A, KCNRG-A | 1 | Low |
| 6 | MTERF4-A, TXDNC2-A, BCL7A-G, FIGNL2-A, SULT2B1-G, NOL3-A | 1 | Low |
| 6 | ACPP-A, NUDT14-A, DCLK1-A, SS18-A, TOM1L2-A, NOL3-A | 1 | Low |
| 6 | TRIM33-A, BCL7A-A, CTAG1A-G, MAGEC2-G, MTERF4-A, HRAS-G | 1 | Low |
| 6 | ACPP-A, DCLK1-A, SS18-A, TOM1L2-A, FIGNL2-A, NOL3-A | 1 | Low |
| 6 | TRIM33-A, BCL7A-A, DDX4-G, CTAG1A-G, C1QTNF1-G, SSBP4-A | 1 | Low |
| 6 | CTAG1A-G, TXDNC2-A, BCL7A-G, SULT2B1-G, DCLK1-G, NOL3-A | 1 | Low |
| 6 | BCL7A-A, DDX4-G, CTAG1A-G, MAGEC2-G, APH1A-A, N4BP1-A | 1 | Low |
| 6 | MTERF4-A, RPLP1-A, SS18-A, TOM1L2-A, TTC1-A, NOL3-A | 1 | Low |
| 6 | ACPP-A, NUDT14-A, DCLK1-A, TOM1L2-A, FIGNL2-A, NOL3-A | 1 | Low |
| 6 | BCL7A-A, DDX4-G, CTAG1A-G, MAGEC2-G, NUDT14-A, HRAS-G | 1 | Low |
| 6 | BCL7A-A, DDX4-G, CTAG1A-G, MAGEC2-G, MAGEA8-A, RALGDS-A | 1 | Low |
| 6 | MTERF4-A, RPLP1-A, TOM1L2-A, JAKMIP2-A, SULT2B1-G, NOL3-A | 1 | Low |
| 6 | MAGEC2-G, TRIM33-G, JAKMIP2-A, TXDNC2-A, SULT2B1-G, NOL3-A | 1 | Low |
| 6 | BCL7A-A, DDX4-G, CTAG1A-G, MAGEC2-G, MTERF4-A, NOL3-G | 1 | Low |
| 6 | TRIM33-A, BCL7A-A, CTAG1A-G, MAGEC2-G, MTERF4-A, C1QTNF1-G | 1 | Low |
| 6 | MTERF4-A, RPLP1-A, SS18-A, TOM1L2-A, SPN-A, NOL3-A | 1 | Low |
| 6 | TRIM33-A, BCL7A-A, CTAG1A-G, MAGEC2-G, MTERF4-A, SSBP4-A | 1 | Low |
| 6 | TRIM33-A, MAGEA8-A, MAGEC2-A, TOM1L2-A, JAKMIP2-A, NOL3-A | 1 | Low |
| 6 | ACPP-A, DCLK1-A, RPLP1-A, SS18-A, TOM1L2-A, C14ORF37-A | 1 | Low |
| 6 | BCL7A-A, DDX4-G, CTAG1A-G, MAGEC2-G, MTERF4-A, APH1A-A | 1 | Low |
| 6 | BCL7A-A, DDX4-G, CTAG1A-G, MTERF4-A, YP021-A, PIP4K2C-A | 1 | Low |
| 6 | MAGEC2-G, TOM1L2-A, JAKMIP2-A, TXDNC2-A, SULT2B1-G, NOL3-A | 1 | Low |
| 6 | ACPP-A, NUDT14-A, DCLK1-A, TOM1L2-A, JAKMIP2-A, NOL3-A | 1 | Low |
| 6 | ACPP-A, RPLP1-A, SS18-A, TOM1L2-A, FIGNL2-A, NOL3-A | 1 | Low |
| 6 | ACPP-A, RPLP1-A, TOM1L2-A, C14ORF37-A, JAKMIP2-A, NOL3-A | 1 | Low |
| 6 | ACPP-A, RPLP1-A, TOM1L2-A, TTC1-A, JAKMIP2-A, NOL3-A | 1 | Low |
| 6 | ACPP-A, RPLP1-A, SS18-A, TOM1L2-A, SPN-A, NOL3-A | 1 | Low |
| 6 | TRIM33-A, BCL7A-A, DDX4-G, CTAG1A-G, MTERF4-A, APH1A-A | 1 | Low |
| 6 | MAGEC2-G, JAKMIP2-A, TXDNC2-A, SULT2B1-G, SPN-A, NOL3-A | 1 | Low |
| 6 | TRIM33-A, BCL7A-A, DDX4-G, CTAG1A-G, MTERF4-A, KCNRG-A | 1 | Low |
| 6 | ACPP-A, RPLP1-A, TOM1L2-A, FIGNL2-A, SPN-A, NOL3-A | 1 | Low |
| 6 | BCL7A-A, DDX4-G, CTAG1A-G, MAGEC2-G, MTERF4-A, RALGDS-A | 1 | Low |
| 6 | MTERF4-A, TOM1L2-A, JAKMIP2-A, TXDNC2-A, SULT2B1-G, NOL3-A | 1 | Low |
| 6 | BCL7A-A, DDX4-G, CTAG1A-G, MAGEC2-G, N4BP1-A, HRAS-G | 1 | Low |
| 6 | BCL7A-A, DDX4-G, CTAG1A-G, MTERF4-A, APH1A-A, RALGDS-G | 1 | Low |
| 6 | BCL7A-A, DDX4-G, CTAG1A-G, MAGEC2-G, MTERF4-A, HRAS-G | 1 | Low |
| 6 | MTERF4-A, TOM1L2-A, TTC1-A, JAKMIP2-A, TXDNC2-A, NOL3-A | 1 | Low |
| 6 | BCL7A-A, DDX4-G, CTAG1A-G, MAGEC2-G, SSBP4-A, RALGDS-A | 1 | Low |
| 6 | MAGEC2-G, TEX264-G, TRIM33-G, JAKMIP2-A, SULT2B1-G, NOL3-A | 1 | Low |

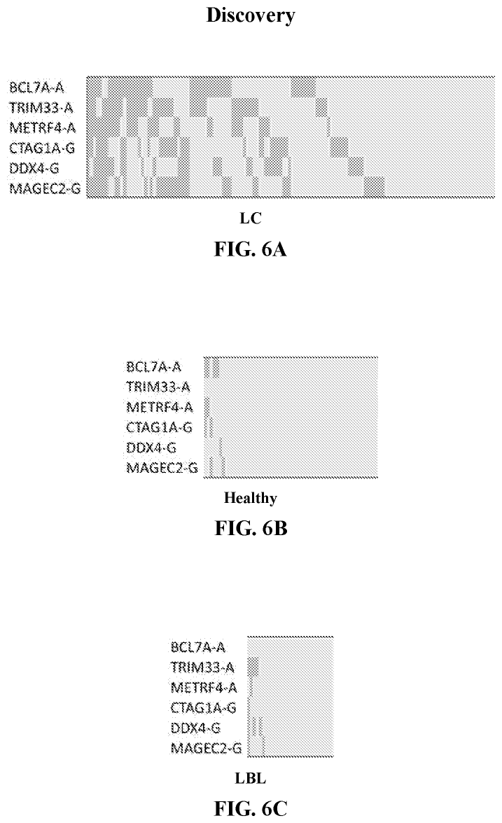

Example data for analysis of panels in Table 7 in the Discovery cohort is shown in FIGS. 6A, 6B, and 6C, using the same ELISA method as described in Example 4. A BCL7A-A, TRIM33-A, MTERF4-A, CTAG1A-G, DDX4-G, and MAGEC2-G panel achieved 73.5% sensitivity at 85.1% specificity with a k value of 1.

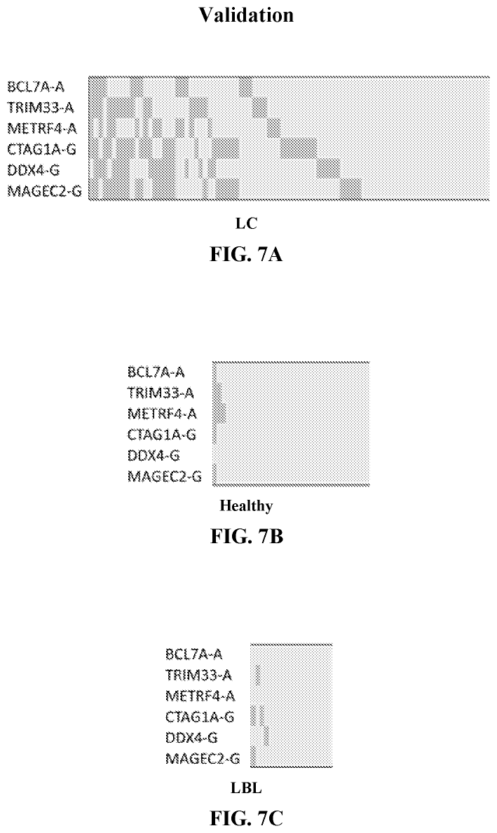

Example data for analysis of the same integrated biomarker panel against an independent validation cohort is shown in FIGS. 7A, 7B, and 7C. 142 serum samples collected from 88 early stage LC, 36 healthy, and 18 LBLs (Table 8), was assembled and tested in a single-blind fashion. Using the same ELISA method as described in Example 4, 68.2% of samples in the early stages of LC were scored as positives, and only 8.3% and 22.2% of healthy and LBL samples were respectively scored as false positives (Table 9).

TABLE 8
Characteristics of samples in Phase II validation cohort

| | Early LC (n = 88) | | | Healthy (n = 36) | | | LBL (n = 18) | | | |
|---|---|---|---|---|---|---|---|---|---|---|
| Variable | No. | Mean | % | No. | Mean | % | No. | Mean | % | P |
| Age (years) | | | | | | | | | | |
| Mean | | 59.6 | | | 54.8 | | | 58.6 | | 0.051 |
| Standard deviation | | 10 | | | 10.6 | | | 9.95 | | |
| Sex | | | | | | | | | | |
| Male | 55 | | 64.7 | 22 | | 61.1 | 12 | | 66.7 | 0.543 |
| Female | 30 | | 35.3 | 14 | | 38.9 | 6 | | 33.3 | |
| Smoking history | | | | | | | | | | |
| (pack-years) | | | | | | | | | | |
| 0 | 26 | | 29.5 | 13 | | 36.1 | 6 | | 33.3 | 0.110 |
| <20 | 12 | | 13.6 | 5 | | 13.9 | 3 | | 16.7 | |
| ≥20 | 50 | | 56.8 | 18 | | 50.0 | 9 | | 50 | |
| Type | | | | | | | | | | |
| Small Cell Lung Cancer | 16 | | 18.2 | | | | | | | |
| Adenocarcinoma | 48 | | 54.5 | | | | | | | |
| Squamous Cell | | | | | | | | | | |
| Carcinoma | 24 | | 27.3 | | | | | | | |

TABLE 9
Performance of a 6-biomarker panel in discovery and validation stages in Phase II of IgA study

| Stage | Performance | | Positive rate | | | | |
|---|---|---|---|---|---|---|---|
| | Sensitivity | Specificity | Adenocarcinoma | Squamous-cell | Small-cell | Healthy | LBL |
| | | | (64) | carcinoma (48) | carcinoma | (58) | (29) |
| | | | | | (24) | | |
| Discovery | 73.5% | 35.1% | 71.9% | 75.0% | 70.0% | 12.1% | 20.7% |
| | Sensitivity | Specificity | Adenocarcinoma | Squamous-cell | Small-cell | Healthy | LBL |
| | | | (48) | carcinoma (24) | carcinoma | (36) | (18) |
| | | | | | (16) | | |
| Validation | 68.2% | 87.0% | 69.3% | 79.2% | 58.9% | 8.3% | 22.2% |

This design possessed and displayed several strengths. First, it employed the most comprehensive human proteome (HuProt) arrays, with >75% coverage of the human proteome, to improve the likelihood of finding potential biomarkers. Second, the 293 LC patients recruited were diagnosed with three LC subtypes at early stages, with the aim of finding robust LC biomarkers. Third, the LBL samples were combined with healthy subjects as negative control groups to enable better discrimination of malignant from benign lesions. Finally, ELISA was used as an independent platform to validate the newly discovered biomarkers and to identify integrated IgA/IgG biomarker panels.

SEQUENCES

| | | |
|---|---|---|
| SEQ ID NO: 1 | amino acid sequence of | MAEAVLRVARRQLSQRGGSGAPILLRQMFEPVSCTFTYL |
| | Homo sapiens ETHE1 | LGDRESREAVLIDPVLETAPRDAQLIKELGLRLLYAVNTH |
| | | CHADHITGSGLLRSLLPGCQSVISRLSGAQADLHIEDGDSI |
| | | RFGRFALETRASPGHTPGCVTFVLNDHSMAFTGDALLIR |
| | | GCGRTDFQQGCAKTLYHSVHEKIFTLPGDCLIYPAHDYH |
| | | GFTVSTVEEERTLNPRLTLSCEEFVKIMGNLNLPKPQQID |
| | | FAVPANMRCGVQTPTA |
| SEQ ID NO: 2 | amino acid sequence of | MSTPAVPQDLQLPPSQRAQSAFKEQRRQKLKEHLLRRKT |
| | Homo sapiens CKAP2 | LFAYKQENEMLSSRDQRVVTSEDQVQEGTKVLKLKTKM |
| | | ADKENMKRPAESKNNTVVGKHCIPLKPSNELTNSTVVID |
| | | THKPKDSNQTPHLLLTEDDPQSQHMTLSQAFHLKNNSKK |
| | | KQMTTEKQKQDANMPKKPVLGSYRGQIVQSKINSFRKPL |
| | | QVKDESSAATKKLSATIPKATKPQPVNTSSVTVKSNRSSN |
| | | MTATTKFVSTTSQNTQLVRPPIRSHHSNTRDTVKQGISRT |
| | | SANVTIRKGPHEKELLQSKTALSSVKTSSSQGIIRNKTLSR |
| | | SIASEVVARPASLSNDKLMEKSEPVDQRRHTAGKAIVDS |
| | | RSAQPKETSEERKARLSEWKAGKGRVLKRPPNSVVTQH |
| | | EPAGQNEKPVGSFWTTMAEEDEQRLFTEKVNNTFSECLN |
| | | LINEGCPKEDILVTLNDLIKNIPDAKKLVKYWICLALIEPIT |
| | | SPIENIIAIYEKAILAGAQPIEEMRHTIVDILTMKSQEKANL |
| | | GENMEKSCASKEEVKEVSIEDTGVDVDPEKLEMESKLHR |
| | | NLLFQDCEKEQDNKTKDPTHDVKTPNTETRTSCLIKYNV |
| | | STTPYLQSVKKKVQFDGTNSAFKELKFLTPVRRSRRLQE |
| | | KTSKLPDMLKDHYPCVSSLEQLTELGRETDAFVCRPNAA |
| | | LCRVYYEADTT |
| SEQ ID NO: 3 | amino acid sequence of | MKTPWKVLLGLLGAAALVTIITVPVVLLNKGTDDATAD |
| | Homo sapiens DPP4 | SRKTYTLTDYLKNTYRLKLYSLRWISDHEYLYKQENNIL |
| | | VFNAEYGNSSVFLENSTFDEFGHSINDYSISPDGQFILLEY |
| | | NYVKQWRHSYTASYDIYDLNKRQLITEERIPNNTQWVT |
| | | WSPVGHKLAYVWNNDIYVKIEPNLPSYRITWTGKEDIIY |
| | | NGITDWVYEEEVFSAYSALWWSPNGTFLAYAQFNDTEV |
| | | PLIEYSFYSDESLQYPKTVRVPYPKAGAVNPTVKFFVVNT |
| | | DSLSSVTNATSIQITAPASMLIGDHYLCDVTWATQERISL |
| | | QWLRRIQNYSVMDICDYDESSGRWNCLVARQHIEMSTT |
| | | GWVGRFRPSEPHFTLDGNSFYKIISNEEGYRHICYFQIDK |
| | | KDCTFITKGTWEVIGIEALTSDYLYYISNEYKGMPGGRNL |
| | | YKIQLSDYTKVTCLSCELNPERCQYYSVSFSKEAKYYQL |
| | | RCSGPGLPLYTLHSSVNDKGLRVLEDNSALDKMLQNVQ |
| | | MPSKKLDFIILNETKFWYQMILPPHFDKSKKYPLLLDVYA |
| | | GPCSQKADTVFRLNWATYLASTENIIVASFDGRGSGYQG |
| | | DKIMHAINRRLGTFEVEDQIEAARQFSKMGFVDNKRIAI |
| | | WGWSYGGYVTSMVLGSGSGVFKCGIAVAPVSRWEYYD |
| | | SVYTERYMGLPTPEDNLDHYRNSTVMSRAENFKQVEYL |
| | | LIHGTADDNVHFQQSAQISKALVDVGVDFQAMWYTDED |
| | | HGIASSTAHQHIYTHMSHFIKQCFSLP |
| SEQ ID NO: 4 | amino acid sequence of | MPFPFGKSHKSPADIVKNLKESMAVLEKQDISDKKAEKA |
| | Homo sapiens CAB39 | TEEVSKNLVAMKEILYGTNEKEPQTEAVAQLAQELYNSG |
| | | LLSTLVADLQLIDFEGKKDVAQIFNNILRRQIGTRTPTVEY |
| | | ICTQQNILFMLLKGYESPEIALNCGIMLRECIRHEPLAKIIL |
| | | WSEQFYDFFRYVEMSTFDIASDAFATFKDLLTRHKLLSA |
| | | EFLEQHYDRFFSEYEKLLHSENYVTKRQSLKLLGELLLD |
| | | RHNFTIMTKYISKPENLKLMMNLLRDKSRNIQFEAFHVF |
| | | KVFVANPNKTQPILDILLKNQAKLIEFLSKFQNDRTEDEQ |
| | | FNDEKTYLVKQIRDLKRPAQQEA |
| SEQ ID NO: 5 | amino acid sequence of | MEGAGAGSGFRKELVSRLLHLHFKDDKTKEAAVRGVRQ |
| | Homo sapiens STRA13 | AQAEDALRADVDQLEKVLPQLLLDF |
| SEQ ID NO: 6 | amino acid sequence of | MEEPQSDPSVEPPLSQETFSDLWKLLPENNVLSPLPSQAM |
| | Homo sapiens p53 | DDLMLSPDDIEQWFTEDPGPDEAPRMPEAAPRVAPAPAA |
| | | PTPAAPAPAPSWPLSSSVPSQKTYQGSYGFRLGFLHSGTA |
| | | KSVTCTYSPALNKMFCQLAKTCPVQLWVDSTPPPGTRVR |
| | | AMAIYKQSQHMTEVVRRCPHHERCSDSDGLAPPQHLIRV |
| | | EGNLRVEYLDDRNTFRHSVVVPYEPPEVGSDCTTIHYNY |
| | | MCNSSCMGGMNRRPILTIITLEDSSGNLLGRNSFEVRVCA |
| | | CPGRDRRTEEENLRKKGEPHHELPPGSTKRALPNNTSSSP |
| | | QPKKKPLDGEYFTLQIRGRERFEMFRELNEALELKDAQA |
| | | GKEPGGSRAHSSHLKSKKGQSTSRHKKLMFKTEGPDSD |
| SEQ ID NO: 7 | amino acid sequence of | MQAEGRGTGGSTGDADGPGGPGIPDGPGGNAGGPGEAG |
| | Homo sapiens | ATGGRGPRGAGAARASGPGGGAPRGPHGGAASGLNGCC |
| | CTAG1A | RCGARGPESRLLEFYLAMPFATPMEAELARRSLAQDAPP |
| | | LPVPGVLLKEFTVSGNILTIRLTAADHRQLQLSISSCLQQL |
| | | SLLMWITQCFLPVFLAQPPSGQRR |
| SEQ ID NO: 8 | amino acid sequence of | MGSRGQGLLLAYCLLLAFASGLVLSRVPHVQGEQQEWE |
| | Homo sapiens | GTEELPSPPDHAERAEEQHEKYRPSQDQGLPASRCLRCC |
| | C1QTNF1 | DPGTSMYPATAVPQINITILKGEKGDRGDRGLQGKYGKT |
| | | GSAGARGHTGPKGQKGSMGAPGERCKSHYAAFSVGRK |
| | | KPMHSNHYYQTVIFDTEFVNLYDHFNMFTGKFYCYVPG |
| | | LYFFSLNVHTWNQKETYLHIMKNEEEVVILFAQVGDRSI |
| | | MQSQSLMLELREQDQVWVRLYKGERENAIFSEELDTYIT |
| | | FSGYLVKHATEP |
| SEQ ID NO: 9 | amino acid sequence of | MSDLLLLGLIGGLTLLLLLTLLAFAGYSGLLAGVEVSAGS |
| | Homo sapiens TEX264 | PPIRNVTVAYKFHMGLYGETGRLFTESCSISPKLRSIAVY |
| | | YDNPHMVPPDKCRCAVGSILSEGEESPSPELIDLYQKFGF |
| | | KVFSFPAPSHVVTATFPYTTILSIWLATRRVHPALDTYIKE |
| | | RKLCAYPRLEIYQEDQIHFMCPLARQGDFYVPEMKETEW |
| | | KWRGLVEAIDTQVDGTGADTMSDTSSVSLEVSPGSRETS |
| | | AATLSPGASSRGWDDGDTRSEHSYSESGASGSSFEELDLE |
| | | GEGPLGESRLDPGTEPLGTTKWLWEPTAPEKGKE |
| SEQ ID NO: 10 | amino acid sequence of | MASLGLQLVGYILGLLGLLGTLVAMLLPSWKTSSYVGAS |
| | Homo sapiens CLDN2 | IVTAVGFSKGLWMECATHSTGITQCDIYSTLLGLPADIQA |
| | | AQAMMVTSSAISSLACIISVVGMRCTVFCQESRAKDRVA |
| | | VAGGVFFILGGLLGFIPVAWNLHGILRDFYSPLVPDSMKF |
| | | EIGEALYLGIISSLFSLIAGIILCFSCSSQRNRSNYYDAYQA |
| | | QPLATRSSPRPGQPPKVKSEFNSYSLTGYV |
| SEQ ID NO: 11 | amino acid sequence of | MVKLGNNFAEKGTKQPLLEDGFDTIPLMTPLDVNQLQFP |
| | Homo sapiens NSG1 | PPDKVVVKTKTEYEPDRKKGKARPPQIAEFTVSITEGVTE |
| | | RFKVSVLVLFALAFLTCVVFLVVYKVYKYDRACPDGFV |
| | | LKNTQCIPEGLESYYAEQDSSAREKFYTVINHYNLAKQSI |
| | | TRSVSPWMSVLSEEKLSEQETEAAEKSA |
| SEQ ID NO: 12 | amino acid sequence of | MTEYKLVVVGAGGVGKSALTIQLIQNHFVDEYDPTIEDS |
| | Homo sapiens HRas | YRKQVVIDGETCLLDILDTAGQEEYSAMRDQYMRTGEG |
| | | FLCVFAINNTKSFEDIHQYREQIKRVKDSDDVPMVLVGN |
| | | KCDLAARTVESRQAQDLARSYGIPYIETSAKTRQGVEDA |
| | | FYTLVREIRQHKLRKLNPPDESGPGCMSCKCVLSMTEYK |
| | | LVVVGAGGVGKSALTIQLIQNHFVDEYDPTIEDSYRKQV |
| | | VIDGETCLLDILDTAGQEEYSAMRDQYMRTGEGFLCVFA |
| | | INNTKSFEDIHQYREQIKRVKDSDDVPMVLVGNKCDLAA |
| | | RTVESRQAQDLARSYGIPYIETSAKTRQGSRSGSSSSSGTL |
| | | WDPPGPM |
| SEQ ID NO: 13 | amino acid sequence of | MANALASATCERCKGGFAPAEKIVNSNGELYHEQCFVC |
| | Homo sapiens LIMS1 | AQCFQQFPEGLFYEFEGRKYCEHDFQMLFAPCCHQCGEF |
| | | TIGRVIKAMNNSWHPECFRCDLCQEVLADIGFVKNAGRH |
| | | LCRPCHNREKARGLGKYICQKCHAIIDEQPLIFKNDPYHP |
| | | DHFNCANCGKELTADARELKGELYCLPCHDKMGVPICG |
| | | ACRRPIEGRVVNAMGKQWHVEHFVCAKCEKPFLGHRH |
| | | YERKGLAYCETHYNQLFGDVCFHCNRVIEGGVVSALNK |
| | | AWCVNCFACSTCNTKLTLKNKFVEFDMKPVCKKCYEKF |
| | | PLELKKRLKKLAETLGRK |
| SEQ ID NO: 14 | amino acid sequence of | MDHTEGSPAEEPPAHAPSPGKFGERPPPKRLTREAMRNY |
| | Homo sapiens RBPJ1 | KERGDQTVLILHAKVAQKSYGNEKRFFCPPPCVYLMGSG |
| | | WKKKKEQMERDGCSEQESQPCAFIGIGNSDQEMQQLNL |
| | | EGKNYCTAKTLYISDSDKRKHFMLSVKMFYGNSDDIGVF |
| | | LSKRIKVISKPSKKKQSLKNADLCIASGTKVALFNRLRSQ |
| | | TVSTRYLHVEGGNFHASSQQWGAFFIHLLDDDESEGEEF |
| | | TVRDGYIHYGQTVKLVCSVTGMALPRLIIRKVDKQTALL |
| | | DADDPVSQLHKCAFYLKDTERMYLCLSQERIIQFQATPC |
| | | PKEPNKEMINDGASWTIISTDKAEYTFYEGMGPVLAPVT |
| | | PVPVVESLQLNGGGDVAMLELTGQNFTPNLRVWFGDVE |
| | | AETMYRCGESMLCVVPDISAFREGWRWVRQPVQVPVTL |
| | | VRNDGIIYSTSLTFTYTPEPGPRPHCSAAGAILRANSSQVP |
| | | PNESNTNSEGSYTNASTNSTSVTSSTATVVS |
| SEQ ID NO: 15 | amino acid sequence of | MAPVVTGKFGERPPPKRLTREAMRNYLKERGDQTVLILH |
| | Homo sapiens RBPJ2 | AKVAQKSYGNEKRFFCPPPCVYLMGSGWKKKKEQMER |
| | | DGCSEQESQPCAFIGIGNSDQEMQQLNLEGKNYCTAKTL |
| | | YISDSDKRKHFMLSVKMFYGNSDDIGVFLSKRIKVISKPS |
| | | KKKQSLKNADLCIASGTKVALFNRLRSQTVSTRYLHVEG |
| | | GNFHASSQQWGAFFIHLLDDDESEGEEFTVRDGYIHYGQ |
| | | TVKLVCSVTGMALPRLIIRKVDKQTALLDADDPVSQLHK |
| | | CAFYLKDTERMYLCLSQERIIQFQATPCPKEPNKEMINDG |
| | | ASWTIISTDKAEYTFYEGMGPVLAPVTPVPVVESLQLNG |
| | | GGDVAMLELTGQNFTPNLRVWFGDVEAETMYRCGESM |
| | | LCVVPDISAFREGWRWVRQPVQVPVTLVRNDGIIYSTSL |
| | | TFTYTPEPGPRPHCSAAGAILRANSSQVPPNESNTNSEGS |
| | | YTNASTNSTSVTSSTATVVS |
| SEQ ID NO: 16 | amino acid sequence of | MGGCRKFGERPPPKRLTREAMRNYLKERGDQTVLILHA |
| | Homo sapiens RBPJ3 | KVAQKSYGNEKRFFCPPPCVYLMGSGWKKKKEQMERD |
| | | GCSEQESQPCAFIGIGNSDQEMQQLNLEGKNYCTAKTLYI |
| | | SDSDKRKHFMLSVKMFYGNSDDIGVFLSKRIKVISKPSKK |
| | | KQSLKNADLCIASGTKVALFNRLRSQTVSTRYLHVEGGN |
| | | FHASSQQWGAFFIHLLDDDESEGEEFTVRDGYIHYGQTV |
| | | KLVCSVTGMALPRLIIRKVDKQTALLDADDPVSQLHKCA |
| | | FYLKDTERMYLCLSQERIIQFQATPCPKEPNKEMINDGAS |
| | | WTIISTDKAEYTFYEGMGPVLAPVTPVPVVESLQLNGGG |
| | | DVAMLELTGQNFTPNLRVWFGDVEAETMYRCGESMLC |
| | | VVPDISAFREGWRWVRQPVQVPVTLVRNDGIIYSTSLTFT |
| | | YTPEPGPRPHCSAAGAILRANSSQVPPNESNTNSEGSYTN |
| | | ASTNSTSVTSSTATVVS |
| SEQ ID NO: 17 | amino acid sequence of | MQSDDVIWDTLGNKQFCSFKIRTKTQSFCRNEYSLTGLC |
| | Homo sapiens MAK 16 | NRSSCPLANSQYATIKEEKGQCYLYMKVIERAAFPRRLW |
| | | ERVRLSKNYEKALEQIDENLIYWPRFIRHKCKQRFTKITQ |
| | | YLIRIRKLTLKRQRKLVPLSKKVERREKRREEKALIAAQL |
| | | DNAIEKELLERLKQDTYGDIYNFPIHAFDKALEQQEAESD |
| | | SSDTEEKDDDDDDEEDVGKREFVEDGEVDESDISDFEDM |
| | | DKLDASSDEDQDGKSSSEEEEEKALSAKHKGKMPLRGPL |
| | | QRKRAYVEIEYEQETEPVAKAKTT |
| SEQ ID NO: 18 | amino acid sequence of | MEETIKDPPTSAVLLDHCHFSQVIFNSVEKFYIPGGDVTC |
| | Homo sapiens | HYTFTQHFIPRRKDWIGIFRVGWKTTREYYTFMWVTLPI |
| | CALCOCO2 | DLNNKSAKQQEVQFKAYYLPKDDEYYQFCYVDEDGVV |
| | | RGASIPFQFRPENEEDILVVTTQGEVEEIEQHNKELCKEN |
| | | QELKDSCISLQKQNSDMQAELQKKQEELETLQSINKKLE |
| | | LKVKEQKDYWETELLQLKEQNQKMSSENEKMGIRVDQL |
| | | QAQLSTQEKEMEKLVQGDQDKTEQLEQLKKENDHLFLS |
| | | LTEQRKDQKKLEQTVEQMKQNETTAMKKQQELMDENF |
| | | DLSKRLSENEIICNALQRQKERLEGENDLLKRENSRLLSY |
| | | MGLDFNSLPYQVPTSDEGGARQNPGLAYGNPYSGIQESS |
| | | SASPLSIKKCPICKADDICDHTLEQQQMQPLCFNCPICDKI |
| | | FPATEKQIFEDHVFCHSL |
| SEQ ID NO: 19 | amino acid sequence of | MSLYPSLEDLKVDKVIQAQTAFSANPANPAILSEASAPIP |
| | Homo sapiens SDCBP | HDGNLYPRLYPELSQYMGLSLNEEEIRANVAVVSGAPLQ |
| | | GQLVARPSSINYMVAPVTGNDVGIRRAEIKQGIREVILCK |
| | | DQDGKIGLRLKSIDNGIFVQLVQANSPASLVGLRFGDQV |
| | | LQINGENCAGWSSDKAHKVLKQAFGEKITMTIRDRPFER |
| | | TITMHKDSTGHVGFIFKNGKITSIVKDSSAARNGLLTEHNI |
| | | CEINGQNVIGLKDSQIADILSTSGTVVTITIMPAFIFEHIIKR |
| | | MAPSIMKSLMDHTIPEV |
| SEQ ID NO: 20 | amino acid sequence of | MMLSLNNLQNIIYNPVIPFVGTIPDQLDPGTLIVIRGHVPS |
| | Homo sapiens LGALS8 | DADRFQVDLQNGSSMKPRADVAFHFNPRFKRAGCIVCN |
| | | TLINEKWGREEITYDTPFKREKSFEIVIMVLKDKFQVAVN |
| | | GKHTLLYGHRIGPEKIDTLGIYGKVNIHSIGFSFSSDLQST |
| | | QASSLELTEISRENVPKSGTPQLPSNRGGDISKIAPRTVYT |
| | | KSKDSTVNHTLTCTKIPPMNYVSKRLPFAARLNTPMGPG |
| | | RTVVVKGEVNANAKSFNVDLLAGKSKDIALHLNPRLNIK |
| | | AFVRNSFLQESWGEEERNITSFPFSPGMYFEMIIYCDVREF |
| | | KVAVNGVHSLEYKHRFKELSSIDTLEINGDIHLLEVRSW |
| SEQ ID NO: 21 | amino acid sequence of | MADPQAGSAAGDWEIDVESLELEEDVCGAPRSTPPGPSP |
| | Homo sapiens DMRT2 | PPADGDCEDDEDDDGVDEDAEEEGDGEEAGASPGMPGQ |
| | | PEQRGGPQPRPPLAPQASPAGTGPRERCTPAGGGAEPRK |
| | | LSRTPKCARCRNHGVVSCLKGHKRFCRWRDCQCANCLL |
| | | VVERQRVMAAQVALRRQQATEDKKGLSGKQNNFERKA |
| | | VYQRQVRAPSLLAKSILEVLLGLFYSYYVYIMNHL |
| SEQ ID NO: 22 | antigenic sequence for | AVLRVARR |
| | ETHE1 | |
| SEQ ID NO: 23 | antigenic sequence for | GAPILLR |
| | ETHE1 | |
| SEQ ID NO: 24 | antigenic sequence for | MFEPVSCTFTYLL |
| | ETHE1 | |
| SEQ ID NO: 25 | antigenic sequence for | REAVLIDPVLE |
| | ETHE1 | |
| SEQ ID NO: 26 | antigenic sequence for | HTPGCVTFVLN |
| | ETHE1 | |
| SEQ ID NO: 27 | antigenic sequence for | TGDALLIR |
| | ETHE1 | |
| SEQ ID NO: 28 | antigenic sequence for | NPRLTLSCEEFVKI |
| | ETHE1 | |
| SEQ ID NO: 29 | antigenic sequence for | NLPKPQQIDFAVPA |
| | ETHE1 | |
| SEQ ID NO: 30 | antigenic sequence for | PAVPQDLQLPPSQRAQS |
| | CKAP2 | |
| SEQ ID NO: 31 | antigenic sequence for | LKEHLLRR |
| | CKAP2 | |
| SEQ ID NO: 32 | antigenic sequence for | RDQRVVTSE |
| | CKAP2 | |
| SEQ ID NO: 33 | antigenic sequence for | EGTKVLKL |
| | CKAP2 | |
| SEQ ID NO: 34 | antigenic sequence for | NNTVVGKHCIPL |
| | CKAP2 | |
| SEQ ID NO: 35 | antigenic sequence for | TNSTVVID |
| | CKAP2 | |
| SEQ ID NO: 36 | antigenic sequence for | QTPHLLLT |
| | CKAP2 | |
| SEQ ID NO: 37 | antigenic sequence for | QHMTLSQAF |
| | CKAP2 | |
| SEQ ID NO: 38 | antigenic sequence for | KKPVLGS |
| | CKAP2 | |
| SEQ ID NO: 39 | antigenic sequence for | RGQIVQS |
| | CKAP2 | |
| SEQ ID NO: 40 | antigenic sequence for | RKPLQVKD |
| | CKAP2 | |
| SEQ ID NO: 41 | antigenic sequence for | ATKKLSATI |
| | CKAP2 | |
| SEQ ID NO: 42 | antigenic sequence for | KPQPVNTSSVTV |
| | CKAP2 | |
| SEQ ID NO: 43 | antigenic sequence for | TTKFVST |
| | CKAP2 | |
| SEQ ID NO: 44 | antigenic sequence for | NTQLVRPPIRS |
| | CKAP2 | |
| SEQ ID NO: 45 | antigenic sequence for | EKELLQSKTALSSVKT |
| | CKAP2 | |
| SEQ ID NO: 46 | antigenic sequence for | IASEVVARPAS |
| | CKAP2 | |
| SEQ ID NO: 47 | antigenic sequence for | AGKAIVDS |
| | CKAP2 | |
| SEQ ID NO: 48 | antigenic sequence for | PPNSVVTQ |
| | CKAP2 | |
| SEQ ID NO: 49 | antigenic sequence for | PKEDILVTLN |
| | CKAP2 | |
| SEQ ID NO: 50 | antigenic sequence for | DAKKLVKYWICLALIEPIT |
| | CKAP2 | |
| SEQ ID NO: 51 | antigenic sequence for | IENIIAIYEKAILAGA |
| | CKAP2 | |
| SEQ ID NO: 52 | antigenic sequence for | RHTIVDI |
| | CKAP2 | |
| SEQ ID NO: 53 | antigenic sequence for | EVKEVSIE |
| | CKAP2 | |
| SEQ ID NO: 54 | antigenic sequence for | TGVDVDP |
| | CKAP2 | |
| SEQ ID NO: 55 | antigenic sequence for | LHRNLLFQD |
| | CKAP2 | |
| SEQ ID NO: 56 | antigenic sequence for | FKELKFLTP |
| | CKAP2 | |
| SEQ ID NO: 57 | antigenic sequence for | LKDHYPCVSSLEQ |
| | CKAP2 | |
| SEQ ID NO: 58 | antigenic sequence for | TDAFVCRPNAALCRVYY |
| | CKAP2 | |
| SEQ ID NO: 59 | antigenic sequence for | YRLKLYSLRW |
| | DPP4 | |
| SEQ ID NO: 60 | antigenic sequence for | SDHEYLY |
| | DPP4 | |
| SEQ ID NO: 61 | antigenic sequence for | NNILVFN |
| | DPP4 | |
| SEQ ID NO: 62 | antigenic sequence for | GNSSVFL |
| | DPP4 | |
| SEQ ID NO: 63 | antigenic sequence for | PDGQFILLEYNYVKQ |
| | DPP4 | |
| SEQ ID NO: 58 | antigenic sequence for | HSYTASYDIYD |
| | DPP4 | |
| SEQ ID NO: 64 | antigenic sequence for | WVTWSPVGHKLAY |
| | DPP4 | |
| SEQ ID NO: 65 | antigenic sequence for | WNNDIYVKIEPNLPSY |
| | DPP4 | |
| SEQ ID NO: 66 | antigenic sequence for | YEEEVFSAYSAL |
| | DPP4 | |
| SEQ ID NO: 67 | antigenic sequence for | GTFLAYA |
| | DPP4 | |
| SEQ ID NO: 68 | antigenic sequence for | SLSSVTN |
| | DPP4 | |
| SEQ ID NO: 69 | antigenic sequence for | ERISLQWL |
| | DPP4 | |
| SEQ ID NO: 70 | antigenic sequence for | QNYSVMDICDY |
| | DPP4 | |
| SEQ ID NO: 71 | antigenic sequence for | RWNCLVAR |
| | DPP4 | |
| SEQ ID NO: 72 | antigenic sequence for | YRHICYFQ |
| | DPP4 | |
| SEQ ID NO: 73 | antigenic sequence for | DKKDCTF |
| | DPP4 | |
| SEQ ID NO: 74 | antigenic sequence for | EVIGIEALTSDYLYYIS |
| | DPP4 | |
| SEQ ID NO: 75 | antigenic sequence for | GLRVLED |
| | DPP4 | |
| SEQ ID NO: 76 | antigenic sequence for | MLQNVQMP |
| | DPP4 | |
| SEQ ID NO: 77 | antigenic sequence | KKLDFIIL |
| | foDPP4 | |
| SEQ ID NO: 78 | antigenic sequence for | WYQMILPPH |
| | DPP4 | |
| SEQ ID NO: 79 | antigenic sequence for | WATYLAS |
| | DPP4 | |
| SEQ ID NO: 80 | antigenic sequence for | ENIIVAS |
| | DPP4 | |
| SEQ ID NO: 81 | antigenic sequence for | YYDSVYT |
| | DPP4 | |
| SEQ ID NO: 82 | antigenic sequence for | KQVEYLLIH |
| | DPP4 | |
| SEQ ID NO: 83 | antigenic sequence for | HKSPADIVKN |
| | CAB39 | |
| SEQ ID NO: 84 | antigenic sequence for | ESMAVLEK |
| | CAB39 | |
| SEQ ID NO: 85 | antigenic sequence for | VSKNLVAMKEI |
| | CAB39 | |
| SEQ ID NO: 86 | antigenic sequence for | Q1EAVAQLAQE |
| | CAB39 | |
| SEQ ID NO: 87 | antigenic sequence for | YNSGLLSTLVADLQLID |
| | CAB39 | |
| SEQ ID NO: 88 | antigenic sequence for | KDVAQIFN |
| | CAB39 | |
| SEQ ID NO: 89 | antigenic sequence for | PTVEYICTQQNILFMLLKG |
| | CAB39 | |
| SEQ ID NO: 90 | antigenic sequence for | QFYDFFRYVE |
| | CAB39 | |
| SEQ ID NO: 91 | antigenic sequence for | EYEKLLHSEN |
| | CAB39 | |
| SEQ ID NO: 92 | antigenic sequence for | QSLKLLGELLLD |
| | CAB39 | |
| SEQ ID NO: 93 | antigenic sequence for | QFEAFHVFKVFVA |
| | CAB39 | |
| SEQ ID NO: 94 | antigenic sequence for | TQPILDILLKNQAKLIEFLS |
| | CAB39 | |
| SEQ ID NO: 95 | antigenic sequence for | KTYLVKQI |
| | CAB39 | |
| SEQ ID NO: 96 | antigenic sequence for | RKELVSRLLHLHF |
| | STRA13 | |
| SEQ ID NO: 97 | antigenic sequence for | KEAAVRGVRQ |
| | STRA13 | |
| SEQ ID NO: 98 | antigenic sequence for | LRADVDQLEKVLPQL |
| | STRA13 | |
| SEQ ID NO: 99 | antigenic sequence for | SDPSVEPPLSQ |
| | P53 | |
| SEQ ID NO: 101 | antigenic sequence for | PENNVLSPLPS |
| | P53 | |
| SEQ ID NO: 102 | antigenic sequence for | YGFRLGFLHS |
| | P53 | |
| SEQ ID NO: 103 | antigenic sequence for | TAKSVTCTYSP |
| | P53 | |
| SEQ ID NO: 104 | antigenic sequence for | LNKMFCQLAKTCPVQLWVDS |
| | P53 | |
| SEQ ID NO: 105 | antigenic sequence for | RAMAIYKQ |
| | P53 | |
| SEQ ID NO: 106 | antigenic sequence for | HMIEVVRRCPHHER |
| | P53 | |
| SEQ ID NO: 107 | antigenic sequence for | GLAPPQHLIRV |
| | P53 | |
| SEQ ID NO: 108 | antigenic sequence for | PILTIITL |
| | P53 | |
| SEQ ID NO: 109 | antigenic sequence for | FEVRVCACP |
| | P53 | |
| SEQ ID NO: 110 | antigenic sequence for | PHHELPP |
| | P53 | |
| SEQ ID NO: 111 | antigenic sequence for | DGEYFTL |
| | P53 | |
| SEQ ID NO: 112 | antigenic sequence for | AHSSHLK |
| | P53 | |
| SEQ ID NO: 113 | antigenic sequence for | YKLVVVGA |
| | HRas | |
| SEQ ID NO: 114 | antigenic sequence for | GVGKSALTIQLIQNHFVDE |
| | HRas | |
| SEQ ID NO: 115 | antigenic sequence for | YRKQVVIDGETCLLDI |
| | HRas | |
| SEQ ID NO: 116 | antigenic sequence for | EGFLCVFA |
| | HRas | |
| SEQ ID NO: 117 | antigenic sequence for | FEDIHQY |
| | HRas | |
| SEQ ID NO: 118 | antigenic sequence for | DVPMVLVGNKCDLAAR |
| | HRas | |
| SEQ ID NO: 119 | antigenic sequence for | QAQDLARSYGIPYIE |
| | HRas | |
| SEQ ID NO: 120 | antigenic sequence for | VEDAFYTLVRE |
| | HRas | |
| SEQ ID NO: 121 | amino acid sequence of | MSGRSVRAETRSRAKDDIKRVMAAIEKVRKWEKKWVT |
| | Homo sapiens BCL7A | VGDTSLRIYKWVPVTEPKVDDKNKNKKKGKDEKCGSEV |
| | | TTPENSSSPGMMDMHDDNSNQSSIADASPIKQENSSNSSP |
| | | APEPNSAVPSDGTEAKVDEAQADGKEHPGAEDASDEQN |
| | | SQSSMEHSMNSSEKVDRQPSGDSGLAAETSAISQDLEGV |
| | | PPSKKMKLEASQQNSEEM |
| SEQ ID NO: 122 | amino acid sequence of | MGDEDWEAEINPHMSSYVPIFEKDRYSGENGDNFNRTPA |
| | Homo sapiens DDX4 | SSSEMDDGPSRRDHFMKSGFASGRNFGNRDAGECNKRD |
| | | NTSTMGGFGVGKSFGNRGFSNSRFEDGDSSGFWRESSND |
| | | CEDNPTRNRGFSKRGDNDLDPDECMQRTGGLFGSRRPV |
| | | LSGTGNGDTSQSRSGSGSERGGYKGLNEEVITGSGKNSW |
| | | KSEAEGGESSDTQGPKVTYIPPPPPEDEDSIFAHYQTGINF |
| | | DKYDTILVEVSGHDAPPAILTFEEANLCQTLNNNIAKAGY |
| | | TKLTPVQKYSIPIILAGRDLMACAQTGSGKTAAFLLPILA |
| | | HMMHDGITASRFKELQEPECIIVAPTRELVNKIYLEARKF |
| | | SFGTCVRAVVIYGGTQLGHSIRQIVQGCNILCATPGRLMD |
| | | IIGKEKIGLKQIKYLVLDEADRMLDMGFGPEMKKLISCPG |
| | | MPSKEQRQTLMFSATFPEEIQRLAAEFLKSNYLFVAVGQ |
| | | VGGACRDVQQTVLQVGQFSKREKLVEILRNIGDERTMVF |
| | | VETKKKADFIATFLCQEKISTTSIHGDREQREREQALGDF |
| | | RFGKCPVLVATSVAARGLDIENVQHVINFDLPSTIDEYVH |
| | | RIGRTGRCGNTGRAISFFDLESDNHLAQPLVKVLTDAQQ |
| | | DVPAWLEEIAFSTYIPGFSGSTRGNVFASVDTRKGKSTLN |
| | | TAGFSSSQAPNPVDDESWD |
| SEQ ID NO: 123 | amino acid sequence of | MPPVPGVPFRNADNDSPTSVELEDWVDAQHPTDEEEEEA |
| | Homo sapiens | SSASSTLYLVFSPSSFSTSSSLILGGPEEEEVPSGVIPNLTES |
| | MAGEC2 | IPSSPPQGPPQGPSQSPLSSCCSSFSWSSFSEESSSQKGEDT |
| | | GTCQGLPDSESSFTYTLDEKVAELVEFLLLKYEAEEPVTE |
| | | AEMLMIVIKYKDYFPVILKRAREFMELLFGLALIEVGPDH |
| | | FCVFANTVGLTDEGSDDEGMPENSLLIIILSVIFIKGNCAS |
| | | EEVIWEVLNAVGVYAGREHFVYGEPRELLTKVWVQGH |
| | | YLEYREVPHSSPPYYEFLWGPRAHSESIKKKVLEFLAKLN |
| | | NTVPSSFPSWYKDALKDVEERVQATIDTADDATVMASES |
| | | LSVMSSNVSFSE |
| SEQ ID NO: 124 | amino acid sequence of | MAAFGRQVLDWHRLIPLTWACMARQTPHLGEQRRTTAS |
| | Homo sapiens | LLRKLTTASNGGVIEELSCVRSNNYVQEPECRRNLVQCL |
| | MTERF4 | LEKQGTPVVQGSLELERVMSSLLDMGFSNAHINELLSVR |
| | | RGASLQQLLDIISEFILLGLNPEPVCVVLKKSPQLLKLPIM |
| | | QMRKRSSYLQKLGLGEGKLKRVLYCCPEIFTMRQQDIND |
| | | TVRLLKEKCLFTVQQVTKILHSCPSVLREDLGQLEYKFQ |
| | | YAYFRMGIKHPDIVKSEYLQYSLTKIKQRHIYLERLGRYQ |
| | | TPDKKGQTQIPNPLLKDILRVSEAEFLARTACTSVEEFQV |
| | | FKKLLAREEEESESSTSDDKRASLDEDEDDDDEEDNDED |
| | | DNDEDDDDEDDDEAEDNDEDEDDDEEE |
| SEQ ID NO: 125 | amino acid sequence of | MYAKGGKGSAVPSDSQAREKLALYVYEYLLHIGAQKSA |
| | Homo sapiens SSBP4 | QTFLSEIRWEKNITLGEPPGFLHSWWCVFWDLYCAAPDR |
| | | REACEHSGEAKAFQDYSAAAAPSPVMGSMAPGDTMAA |
| | | GSMAAGFFQGPPGSQPSPHNPNAPMMGPHGQPFMSPRFP |
| | | GGPRPTLRMPSQPPAGLPGSQPLLPGAMEPSPRAQGHPS |
| | | MGGPMQRVTPPRGMASVGPQSYGGGMRPPPNSLAGPGL |
| | | PAMNMGPGVRGPWASPSGNSIPYSSSSPGSYTGPPGGGG |
| | | PPGTPIMPSPGDSTNSSENMYTIMNPIGQGAGRANFPLGP |
| | | GPEGPMAAMSAMEPHHVNGSLGSGDMDGLPKSSPGAV |
| | | AGLSNAPGTPRDDGEMAAAGTFLHPFPSESYSPGMTMSV |
| SEQ ID NO: 126 | amino acid sequence of | MAENKGGGEAESGGGGSGSAPVTAGAAGPAAQEAEPPL |
| | Homo sapiens TRIM33 | TAVLVEEEEEEGGRAGAEGGAAGPDDGGVAAASSGSAQ |
| | | AASSPAASVGTGVAGGAVSTPAPAPASAPAPGPSAGPPP |
| | | GPPASLLDTCAVCQQSLQSRREAEPKLLPCLHSFCLRCLP |
| | | EPERQLSVPIPGGSNGDIQQVGVIRCPVCRQECRQIDLVD |
| | | NYFVKDTSEAPSSSDEKSEQVCTSCEDNASAVGFCVECG |
| | | EWLCKTCIEAHQRVKFTKDHLIRKKEDVSESVGASGQRP |
| | | VFCPVHKQEQLKLFCETCDRLTCRDCQLLEHKEHRYQFL |
| | | EEAFQNQKGAIENLLAKLLEKKNYVHFAATQVQNRIKEV |
| | | NETNKRVEQEIKVAIFTLINEINKKGKSLLQQLENVTKER |
| | | QMKLLQQQNDITGLSRQVKHVMNFTNWAIASGSSTALL |
| | | YSKRLITFQLRHILKARCDPVPAANGAIRFHCDPTFWAKN |
| | | VVNLGNLVIESKPAPGYTPNVVVGQVPPGTNHISKTPGQI |
| | | NLAQLRLQHMQQQVYAQKHQQLQQMRMQQPPAPVPTT |
| | | TTTTQQHPRQAAPQMLQQQPPRLISVQTMQRGNMNCGA |
| | | FQAHQMRLAQNAARIPGIPRHSGPQYSMMQPHLQRQHS |
| | | NPGHAGPFPVVSVHNTTINPTSPTTATMANANRGPTSPSV |
| | | TAIELIPSVTNPENLPSLPDIPPIQLEDAGSSSLDNLLSRYIS |
| | | GSHLPPQPTSTMNPSPGPSALSPGSSGLSNSHTPVRPPSTS |
| | | STGSRGSCGSSGRTAEKTSLSFKSDQVKVKQEPGTEDEIC |
| | | SFSGGVKQEKFEDGRRSACMLSSPESSLTPPLSTNLHLES |
| | | ELDALASLENHVKIEPADMNESCKQSGLSSLVNGKSPIRS |
| | | LMHRSARIGGDGNNKDDDPNEDWCAVCQNGGDLLCCE |
| | | KCPKVFHLTCHVPTLLSFPSGDWICTFCRDIGKPEVEYDC |
| | | DNLQHSKKGKTAQGLSPVDQRKCERLLLYLYCHELSIEF |
| | | QEPVPASIPNYYKIIKKPMDLSTVKKKLQKKHSQHYQIPD |
| | | DFVADVRLIFKNCERFNEMMKVVQVYADTQEINLKADS |
| | | EVAQAGKAVALYFEDKLTEIYSDRTFAPLPEFEQEEDDG |
| | | EVTEDSDEDFIQPRRKRLKSDERPVHIK |
| SEQ ID NO: 127 | amino acid sequence of | MERIEGASVGRCAASPYLRPLTLHYRQNGAQKSWDFMK |
| | Homo sapiens | THDSVTVLLFNSSRRSLVLVKQFRPAVYAGEVERRFPGS |
| | NUDT14 | LAAVDQDGPRELQPALPGSAGVTVELCAGLVDQPGLSLE |
| | | EVACKEAWEECGYHLAPSDLRRVATYWSGVGLTGSRQT |
| | | MFYTEVTDAQRSGPGGGLVEEGELIEVVHLPLEGAQAFA |
| | | DDPDIPKTLGVIFGVSWFLSQVAPNLDLQ |
| SEQ ID NO: 128 | amino acid sequence of | MASVSELACIYSALILHDDEVTVTEDKINALIKAAGVNVE |
| | Homo sapiens RPLP1 | PFWPGLFAKALANVNIGSLICNVGAGGPAPAAGAAPAGG |
| | | PAPSTAAAPAEEKKVEAKKEESEESDDDMGFGLFD |
| SEQ ID NO: 129 | amino acid sequence of | MGAAVFFGCTFVAFGPAFALFLITVAGDPLRVIILVAGAF |
| | Homo sapiens APH1A | FWLVSLLLASVVWFILVHVTDRSDARLQYGLLIFGAAVS |
| | | VLLQEVFRFAYYKLLKKADEGLASLSEDGRSPISIRQMA |
| | | YVSGLSFGIISGVFSVINILADALGPGVVGIHGDSPYYFLT |
| | | SAFLTAAIILLHTFWGVVFFDACERRRYWALGLVVGSHL |
| | | LTSGLTFLNPWYEASLLPIYAVTVSMGLWAFITAGGSLRS |
| | | IQRSLLCKD |
| SEQ ID NO: 130 | amino acid sequence of | MGSRGQGLLLAYCLLLAFASGLVLSRVPHVQGEQQEWE |
| | Homo sapiens | GTEELPSPPDHAERAEEQHEKYRPSQDQGLPASRCLRCC |
| | C1QTNF1 | DPGTSMYPATAVPQINITILKGEKGDRGDRGLQGKYGKT |
| | | GSAGARGHTGPKGQKGSMGAPGERCKSHYAAFSVGRK |
| | | KPMHSNHYYQTVIFDTEFVNLYDHFNMFTGKFYCYVPG |
| | | LYFFSLNVHTWNQKETYLHIMKNEEEVVILFAQVGDRSI |
| | | MQSQSLMLELREQDQVWVRLYKGERENAIFSEELDTYIT |
| | | FSGYLVKHATEP |
| SEQ ID NO: 131 | amino acid sequence of | MVQRMWAEAAGPAGGAEPLFPGSRRSRSVWDAVRLEV |
| | Homo sapiens | GVPDSCPVVLHSFTQLDPDLPRPESSTQEIGEELINGVIYSI |
| | RALGDS | SLRKVQLHHGGNKGQRWLGYENESALNLYETCKVRTV |
| | | KAGTLEKLVEHLVPAFQGSDLSYVTIFLCTYRAFTTTQQ |
| | | VLDLLFKRYGCILPYSDEDGGPQDQLKNAISSILGTWLDQ |
| | | YSEDFCQPPDFPCLKQLVAYVQLNMPGSDLERRAHLLLA |
| | | QLEHSEPIEAEPEALSPVPALKPTPELELALTPARAPSPVP |
| | | APAPEPEPAPTPAPGSELEVAPAPAPELQQAPEPAVGLES |
| | | APAPALELEPAPEQDPAPSQTLELEPAPAPVPSLQPSWPSP |
| | | VVAENGLSEEKPHLLVFPPDLVAEQFTLMDAELFKKVVP |
| | | YHCLGSIWSQRDKKGKEHLAPTIRATVTQFNSVANCVIT |
| | | TCLGNRSTKAPDRARVVEHWIEVARECRILKNFSSLYAIL |
| | | SALQSNSIHRLKKTWEDVSRDSFRIFQKLSEIFSDENNYSL |
| | | SRELLIKEGTSKFATLEMNPKRAQKRPKETGIIQGTVPYL |
| | | GTFLTDLVMLDTAMKDYLYGRLINFEKRRKEFEVIAQIK |
| | | LLQSACNNYSIAPDEQFGAWFRAVERLSETESYNLSCELE |
| | | PPSESASNTLRTKKNTAIVKRWSDRQAPSTELSTSGSSHS |
| | | KSCDQLRCGPYLSSGDIADALSVHSAGSSSSDVEEINISFV |
| | | PESPDGQEKKFWESASQSSPETSGISSASSSTSSSSASTTPV |
| | | AATRTHKRSVSGLCNSSSALPLYNQQVGDCCIIRVSLDV |
| | | DNGNMYKSILVTSQDKAPAVIRKAMDKHNLEEEEPEDY |
| | | ELLQILSDDRKLKIPENANVFYAMNSTANYDFVLKKRTF |
| | | TKGVKVKHGASSTLPRMKQKGLKIAKGIF |
| SEQ ID NO:132 | amino acid sequence of | MAARAVLDEFTAPAEKAELLEQSRGRIEGLFGVSLAVLG |
| | Homo sapiens N4BP1 | ALGAEEPLPARIWLQLCGAQEAVHSAKEYIKGICEPELEE |
| | | RECYPKDMHCIFVGAESLFLKSLIQDTCADLCILDIGLLGI |
| | | RGSAEAVVMARSHIQQFVKLFENKENLPSSQKESEVKRE |
| | | FKQFVEAHADNYTMDLLILPTSLKKELLTLTQGEENLFET |
| | | GDDEVIEMRDSQQTEFTQNAATGLNISRDETVLQEEARN |
| | | KAGTPVSELTKQMDTVLSSSPDVLFDPINGLTPDEEALSN |
| | | ERICQKRRFSDSEERHTKKQFSLENVQEGEILHDAKTLAG |
| | | NVIADLSDSSADSENLSPDIKETTEEMEYNILVNFFKTMG |
| | | YSQEIVEKVIKVYGPSTEPLLLLEEIEKENKRFQEDREFSA |
| | | GTVYPETNKTKNKGVYSSTNELTTDSTPKKTQAHTQQN |
| | | MVEKFSQLPFKVEAKPCTSNCRINTFRTVPIEQKHEVWGS |
| | | NQNYICNTDPETDGLSPSVASPSPKEVNFVSRGASSHQPR |
| | | VPLFPENGLHQQPEPLLPNNMKSACEKRLGCCSSPHSKP |
| | | NCSTLSPPMPLPQLLPSVTDARSAGPSDHIDSSVTGVQRF |
| | | RDTLKIPYKLELKNEPGRTDLKHIVIDGSNVAITHGLKKF |
| | | FSCRGIAIAVEYFWKLGNRNITVFVPQWRTRRDPNVTEQ |
| | | HFLTQLQELGILSLTPARMVFGERIASHDDRFLLHLADKT |
| | | GGIIVTNDNFREFVNESVSWREIITKRLLQYTFVGDIFMVP |
| | | DDPLGRSGPRLEEFLQKEVCLRDMQPLLSALPNVGMFDP |
| | | SFRVPGTQAASTSHQPPTRIQGAPSSHWLPQQPHFPLLPA |
| | | LPSLQQNLPMPAQRSSAETNELREALLKIFPDSEQRLKID |
| | | QILVAHPYMKDLNALSAMVLD |
| SEQ ID NO: 133 | amino acid sequence of | MGDLPWAPPEAQAPSTAGAGDVAEHQVAPARFLQGAW |
| | Homo sapiens YP021 | RQAAGWLCRETGAAPGSAQAGPPETAHAADPQPRGPQA |
| | | PPRLPPSLSPERVHPGQPAAPAEPAPGAPALRSGPSQPRGL |
| | | RLPVPVPACAGSSAPGSPAALPDSYPWPPPARNRPATLPP |
| | | TSRVSPLAAFLASAPQR |
| SEQ ID NO: 134 | amino acid sequence of | MSSQELVTLNVGGKIFTTRFSTIKQFPASRLARMLDGRDQ |
| | Homo sapiens KCNRG | EFKMVGGQIFVDRDGDLFSFILDFLRTHQLLLPTEFSDYL |
| | | RLQREALFYELRSLVDLLNPYLLQPRPALVEVHFLSRNTQ |
| | | AFFRVFGSCSKTIEMLTGRITVFTEQPSAPTWNGNFFPPQ |
| | | MTLLPLPPQRPSYHDLVFQCGSDSTTDNQTGVRYVSIKP |
| | | DNRKLANGTNVLGLLIDTLLKEGFHLVSTRTVSSEDKTE |
| | | CYSFERIKSPEVLITNETPKPETIIIPEQSQIKK |
| SEQ ID NO: 135 | amino acid sequence of | MLLGQKSQRYKAEEGLQAQGEAPGLMDVQIPTAEEQKA |
| | Homo sapiens | ASSSSTLIMGTLEEVTDSGSPSPPQSPEGASSSLTVTDSTL |
| | MAGEA8 | WSQSDEGSSSNEEEGPSTSPDPAHLESLFREALDEKVAEL |
| | | VRFLLRKYQIKEPVTKAEMLESVIKNYKNHFPDIFSKASE |
| | | CMQVIFGIDVKEVDPAGHSYILVTCLGLSYDGLLGDDQS |
| | | TPKTGLLIIVLGMILMEGSRAPEEAIWEALSVMGLYDGRE |
| | | HSVYWKLRKLLTQEWVQENYLEYRQAPGSDPVRYEFL |
| | | WGPRALAETSYVKVLEHVVRVNARVRISYPSLHEEALGE |
| | | EKGV |
| SEQ ID NO: 136 | amino acid sequence of | MNKLRQSLRRRKPAYVPEASRPHQWQADEDAVRKGTCS |
| | Homo sapiens NUMBL | FPVRYLGHVEVEESRGMHVCEDAVKKLKAMGRKSVKS |
| | | VLWVSADGLRVVDDKTKDLLVDQTIEKVSFCAPDRNLD |
| | | KAFSYICRDGTTRRWICHCFLALKDSGERLSHAVGCAFA |
| | | ACLERKQRREKECGVTAAFDASRTSFAREGSFRLSGGGR |
| | | PAEREAPDKKKAEAAAAPTVAPGPAQPGHVSPTPATTSP |
| | | GEKGEAGTPVAAGTTAAAIPRRHAPLEQLVRQGSFRGFP |
| | | ALSQKNSPFKRQLSLRLNELPSTLQRRTDFQVKGTVPEM |
| | | EPPGAGDSDSINALCTQISSSFASAGAPAPGPPPATTGTSA |
| | | WGEPSVPPAAAFQPGHKRTPSEAERWLEEVSQVAKAQQ |
| | | QQQQQQQQQQQQQQQQQQAASVAPVPTMPPALQPFPAP |
| | | VGPFDAAPAQVAVFLPPPHMQPPFVPAYPGLGYPPMPRV |
| | | PVVGITPSQMVANAFCSAAQLQPQPATLLGKAGAFPPPAI |
| | | PSAPGSQARPRPNGAPWPPEPAPAPAPELDPFEAQWAAL |
| | | EGKATVEKPSNPFSGDLQKTFEIEL |
| SEQ ID NO: 137 | amino acid sequence of | MHWTPEHAQPLNQWPEQHLDVSSTTPSPAHKLELPPGG |
| | Homo sapiens FIGNL2 | RQRCHYAWAHDDISALTASNLLKRYAEKYSGVLDSPYE |
| | | RPALGGYSDASFLNGAKGDPEPWPGPEPPYPLASLHEGL |
| | | PGTKSGGGGGSGALGGSPVLAGNLPEPLYAGNACGGPS |
| | | AAPEYAAGYGGGYLAPGYCAQTGAALPPPPPAALLQPPP |
| | | PPGYGPSAPLYNYPAGGYAAQPGYGALPPPPGPPPAPYL |
| | | TPGLPAPTPLPAPAPPTAYGFPTAAPGAESGLSLKRKAAD |
| | | EGPEGRYRKYAYEPAKAPVADGASYPAADNGECRGNGF |
| | | RAKPPGAAEEASGKYGGGVPLKVLGSPVYGPQLEPFEKF |
| | | PERAPAPRGGFAVPSGETPKGVDPGALELVTSKMVDCGP |
| | | PVQWADVAGQGALKAALEEELVWPLLRPPAYPGSLRPP |
| | | RTVLLFGPRGAGKALLGRCLATQLGATLLRLRGATLAAP |
| | | GAAEGARLLQAAFAAARCRPPSVLLISELEALLPARDDG |
| | | AAAGGALQVPLLACLDGGCGAGADGVLVVGTTSRPAAL |
| | | DEATRRRFSLRFYVALPDSPARGQILQRALAQQGCALSE |
| | | RELAALVQGTQGFSGGELGQLCQQAAAGAGLPGLQRPL |
| | | SYKDLEAALAKVGPRASAKELDSFVEWDKMYGSGH |
| SEQ ID NO: 138 | amino acid sequence of | MGNAQERPSETIDRERKRLVETLQADSGLLLDALLARGV |
| | Homo sapiens NOL3 | LTGPEYEALDALPDAERRVRRLLLLVQGKGEAACQELLR |
| | | CAQRTAGAPDPAWDWQHVGPGYRDRSYDPPCPGHWTP |
| | | EAPGSGTTCPGLPRASDPDEAGGPEGSEAVQSGTPEEPEP |
| | | ELEAEASKEAEPEPEPEPELEPEAEAEPEPELEPEPDPEPEP |
| | | DFEERDESEDS |
| SEQ ID NO: 139 | amino acid sequence of | MASSSVPPATVSAATAGPGPGFGFASKTKKKHFVQQKV |
| | Homo sapiens | KVFRAADPLVGVFLWGVAHSINELSQVPPPVMLLPDDFK |
| | PIP4K2C | ASSKIKVNNHLFHRENLPSHFKFKEYCPQVFRNLRDRFGI |
| | | DDQDYLVSLTRNPPSESEGSDGRFLISYDRTLVIKEVSSED |
| | | IADMHSNLSNYHQYIVKCHGNTLLPQFLGMYRVSVDNE |
| | | DSYMLVMRNMFSHRLPVHRKYDLKGSLVSREASDKEKV |
| | | KELPTLRDMDFLNKNQKVYIGEEEKKIFLEKLKRDVEFL |
| | | VQLKIMDYSLLLGIHDIIRGSEPEEEAPVREDESEVDGDCS |
| | | LTGPPALVGSYGTSPEGIGGYIHSHRPLGPGEFESFIDVYA |
| | | IRSAEGAPQKEVYFMGLIDILTQYDAKKKAAHAAKTVK |
| | | HGAGAEISTVHPEQYAKRFLDFITNIFA |
| SEQ ID NO: 140 | amino acid sequence of | MSVAFAAPRQRGKGEITPAAIQKMLDDNNHLIQCIMDSQ |
| | Homo sapiens SS18 | NKGKTSECSQYQQMLHTNLVYLATIADSNQNMQSLLPA |
| | | PPIQNMPMGPGGMNQSGPPPPPRSHNMPSDGMVGGGPP |
| | | APHMQNQMNGQMPGPNHMPMQGPGPNQLNMTNSSMN |
| | | MPSSSHGSMGGYNHSVPSSQSMPVQNQMTMSQGQPMG |
| | | NYGPRPNMSMQPNQGPMMHQQPPSQQYNMPQGGGQH |
| | | YQGQQPPMGMMGQVNQGNHMMGQRQIPPYRPPQQGPP |
| | | QQYSGQEDYYGDQYSHGGQGPPEGMNQQYYPDGHNDY |
| | | GYQQPSYPEQGYDRPYEDSSQHYYEGGNSQYGQQQDAY |
| | | QGPPPQQGYPPQQQQYPGQQGYPGQQQGYGPSQGGPGP |
| | | QYPNYPQGQGQQYGGYRPTQPGPPQPPQQRPYGYDQGQ |
| | | YGNYQQ |
| SEQ ID NO: 141 | amino acid sequence of | MRAAPLLLARAASLSLGFLFLLFFWLDRSVLAKELKFVT |
| | Homo sapiens ACPP | LVFRHGDRSPIDTFPTDPIKESSWPQGFGQLTQLGMEQHY |
| | | ELGEYIRKRYRKFLNESYKHEQVYIRSTDVDRTLMSAMT |
| | | NLAALFPPEGVSIWNPILLWQPIPVHTVPLSEDQLLYLPFR |
| | | NCPRFQELESETLKSEEFQKRLHPYKDFIATLGKLSGLHG |
| | | QDLFGIWSKVYDPLYCESVHNFTLPSWATEDTMTKLREL |
| | | SELSLLSLYGIHKQKEKSRLQGGVLVNEILNHMKRATQIP |
| | | SYKKLIMYSAHDTTVSGLQMALDVYNGLLPPYASCHLT |
| | | ELYFEKGEYFVEMHYRNETQHEPYPLMLPGCSPSCPLER |
| | | FAELVGPVIPQDWSTECMTTNSHQGTEDSTD |
| SEQ ID NO: 142 | amino acid sequence of | MDGPAEPQIPGLWDTYEDDISEISQKLPGEYFRYKGVPFP |
| | Homo sapiens | VGLYSLESISLAENTQDVRDDDIFIITYPKSGTTWMIEIICL |
| | SULT2B1 | ILKEGDPSWIRSVPIWERAPWCETIVGAFSLPDQYSPRLM |
| | | SSHLPIQIFTKAFFSSKAKVIYMGRNPRDVVVSLYHYSKI |
| | | AGQLKDPGTPDQFLRDFLKGEVQFGSWFDHIKGWLRMK |
| | | GKDNFLFITYEELQQDLQGSVERICGFLGRPLGKEALGSV |
| | | VAHSTFSAMKANTMSNYTLLPPSLLDHRRGAFLRKGVC |
| | | GDWKNHFTVAQSEAFDRAYRKQMRGMPTFPWDEDPEE |
| | | DGSPDPEPSPEPEPKPSLEPNTSLEREPRPNSSPSPSPGQAS |
| | | ETPHPRPS |
| SEQ ID NO: 143 | amino acid sequence of | MEFLLGNPFSTPVGQCLEKATDGSLQSEDWTLNMEICDII |
| | Homo sapiens | NETEEGPKDAIRALKKRLNGNRNYREVMLALTAWADAF |
| | TOM1L2 | RSSPDLTGVVHIYEELKRKGVEFPMADLDALSPIHTPQRS |
| | | VPEVDPAATMPRSQSQQRTSAGSYSSPPPAPYSAPQAPAL |
| | | SVTGPITANSEQIARLRSELDVVRGNTKVMSEMLTEMVP |
| | | GQEDSSDLELLQELNRTCRAMQQRIVELISRVSNEEVTEE |
| | | LLHVNDDLNNVFLRYERFERYRSGRSVQNASNGVLNEV |
| | | TEDNLIDLGPGSPAVVSPMVGNTAPPSSLSSQLAGLDLGT |
| | | ESVSGTLSSLQQCNPRDGFDMFAQTRGNSLAEQRKTVTY |
| | | EDPQAVGGLASALDNRKQSSEGIPVAQPSVMDDIEVWLR |
| | | TDLKGDDLEEGVTSEEFDKFLEERAKAAEMVPDLPSPPM |
| | | EAPAPASNPSGRKKPERSEDALFAL |
| SEQ ID NO: 144 | amino acid sequence of | MATLLLLLGVLVVSPDALGSTTAVQTPTSGEPLVSTSEPL |
| | Homo sapiens SPN | SSKMYTTSITSDPKADSTGDQTSALPPSTSINEGSPLWTSI |
| | | GASTGSPLPEPTTYQEVSIKMSSVPQETPHATSHPAVPITA |
| | | NSLGSHTVTGGTITTNSPETSSRTSGAPVTTAASSLETSRG |
| | | TSGPPLTMATVSLETSKGTSGPPVTMATDSLETSTGTTGP |
| | | PVTMTTGSLEPSSGASGPQVSSVKLSTMMSPTTSTNASTV |
| | | PFRNPDENSRGMLPVAVLVALLAVIVLVALLLLWRRRQ |
| | | KRRTGALVLSRGGKRNGVVDAWAGPAQVPEEGAVTVT |
| | | VGGSGGDKGSGFPDGEGSSRRPTLTTFFGRRKSRQGSLA |
| | | MEELKSGSGPSLKGEEEPLVASEDGAVDAPAPDEPEGGD |
| | | GAAP |
| SEQ ID NO: 145 | amino acid sequence of | MSFGRDMELEHFDERDKAQRYSRGSRVNGLPSPTHSAH |
| | Homo sapiens DCLK1 | CSFYRTRTLQTLSSEKKAKKVRFYRNGDRYFKGIVYAISP |
| | | DRFRSFEALLADLTRTLSDNVNLPQGVRTIYTIDGLKKISS |
| | | LDQLVEGESYVCGSIEPFKKLEYTKNVNPNWSVNVKTTS |
| | | ASRAVSSLATAKGSPSEVRENKDFIRPKLVTIIRSGVKPRK |
| | | AVRILLNKKTAHSFEQVLTDITDAIKLDSGVVKRLYTLDG |
| | | KQVMCLQDFFGDDDIFIACGPEKFRYQDDFLLDESECRV |
| | | VKSTSYTKIASSSRRSTTKSPGPSRRSKSPASTSSVNGTPG |
| | | SQLSTPRSGKSPSPSPTSPGSLRKQRSSQHGGSSTSLASTK |
| | | VCSSMDENDGPGEEVSEEGFQIPATHERYKVGRTIGDGN |
| | | FAVVKECVERSTAREYALKIIKKSKCRGKEHMIQNEVSIL |
| | | RRVKHPNIVLLIEEMDVPTELYLVMELVKGGDLFDAITST |
| | | NKYTERDASGMLYNLASAIKYLHSLNIVHRDIKPENLLV |
| | | YEHQDGSKSLKLGDFGLATIVDGPLYTVCGTPTYVAPEII |
| | | AETGYGLKVDIWAAGVITYILLCGFPPFRGSGDDQEVLF |
| | | DQILMGQVDFPSPYWDNVSDSAKELITMMLLVDVDQRF |
| | | SAVQVLEHPWVNDDGLPENEHQLSVAGKIKKHFNTGPK |
| | | PNSTAAGVSVIALDHGFTIKRSGSLDYYQQPGMYWIRPP |
| | | LLIRRGRFSDEDATRM |
| SEQ ID NO: 146 | amino acid sequence of | MRGPIVLHICLAFCSLLLFSVATQCLAFPKIERRREIAHVH |
| | Homo sapiens | AEKGQSDKMNTDDLENSSVTSKQTPQLVVSEDPMMMSA |
| | C14orf37 | VPSATSLNKAFSINKETQPGQAGLMQTERPGVSTPTESGV |
| | | PSAEEVFGSSQPERISPESGLAKAMLTIAITATPSLTVDEK |
| | | EELLTSTNFQPIVEEITETTKGFLKYMDNQSFATESQEGV |
| | | GLGHSPSSYVNTKEMLTTNPKTEKFEADTDHRTTSFPGA |
| | | ESTAGSEPGSLTPDKEKPSQMTADNTQAAATKQPLETSE |
| | | YTLSVEPETDSLLGAPEVTVSVSTAVPAASALSDEWDDT |
| | | KLESVSRIRTPKLGDNEETQVRTEMSQTAQVSHEGMEGG |
| | | QPWTEAAQVALGLPEGETHTGTALLIAHGNERSPAFTDQ |
| | | SSFTPTSLMEDMKVSIVNLLQSTGDFTESTKENDALFFLE |
| | | TTVSVSVYESEADQLLGNTMKDIITQEMTTAVQEPDATL |
| | | SMVTQEQVATLELIRDSGKTEEEKEDPSPVSDVPGVTQLS |
| | | RRWEPLATTISTTVVPLSFEVTPTVEEQMDTVTGPNEEFT |
| | | PVLGSPVTPPGIMVGEPSISPALPALEASSERRTVVPSITRV |
| | | NTAASYGLDQLESEEGQEDEDEEDEEDEDEEEEDEEEDE |
| | | EDKDADSLDEGLDGDTELPGFTLPGITSQEPGLEEGNMD |
| | | LLEGATYQVPDALEWEQQNQGLVRSWMEKLKDKAGY |
| | | MSGMLVPVGVGIAGALFILGALYSIKVMNRRRRNGFKR |
| | | HKRKQREFNSMQDRVMLLADSSEDEF |
| SEQ ID NO: 147 | amino acid sequence of | MVSHTFHMRTEESDASQEGDDLPKSSANTSHPKQDDSPK |
| | Homo sapiens | SSEETIQPKEGDIPKAPEETIQSKKEDLPKSSEKAIQPKESN |
| | TXDNC2 | IPKSSAKPIQPKLGNIPKASVKPSQPKEGDIPKAPEETIQSK |
| | | KEDLPKSSEEAIQPKEGDIPKSSAKPIQPKLGNIAKTSVKP |
| | | SQPKESDIPKSPEETIQPKEGDIPKSSAKPIQPKLGNIPKAS |
| | | VKPSQPKEGDISKSPEEAIQPKEGDLPKSLEEAIQPKEGDI |
| | | PKSPEEAIQPKEGDIPKSLEEAIQPKKGDIPKSPEETIQPKK |
| | | DDIPKSPEEAIQPKEGDIPKSPKQAIQPKEGDIPKSLEEAIP |
| | | PKEIDIPKSPEETIQPKEDDSPKSLEEATPSKEGDILKPEEE |
| | | TMEFPEGDKVKVILSKEDFEASLKEAGERLVAVDFSATW |
| | | CGPCRTIRPFFHALSVKHEDVVFLEVDADNCEEVVRECAI |
| | | MCVPTFQFYKKEEKVDELCGALKEKLEAVIAELK |
| SEQ ID NO: 148 | amino acid sequence of | MSKKGRNKGEKPEALIVALQAANEDLRTKLTDIQIELHQ |
| | Homo sapiens | EKSKVSKLEREKTQEAKRIRELEQRKHTVLVTELKAKLH |
| | JAKMIP2 | EEKMKELQAVRENLIKQHEQEMSRTVKVRDGEIQRLKS |
| | | ALCALRDGSSDKVRTALTIEAREEARKLFDTERLKLLQEI |
| | | ADLKTAKKQVDEALSNMIQADKIKAGDLRSEHQSHQEAI |
| | | SKIKWESERDIRRLMDEIKAKDRIIFSLEKELETQTGYVQ |
| | | KLQLQKEALDEQLFLVKEAECNMSSPKREIPGRAGDGSE |
| | | HCSSPDLRRNQKRIAELNATIRKLEDRNTLLGDERNELLK |
| | | RVRETEKQCKPLLERNKCLAKRNDELMVSLQRMEEKLK |
| | | AVTKENSEMREKITSHPPLKKLKSLNDLDQANEEQETEF |
| | | LKLQVIEQQNIIDELTRDREKLIRRRKHRRSSKPIKRPVLD |
| | | PFIGYDEDSMDSETSSMASFRTDRTPATPDDDLDESLAAE |
| | | ESELRFRQLTKEYQALQRAYALLQEQTGGIIDAEREAKA |
| | | QEQLQAEVLRYKAKIEDLEATLAQKGQDSHWVEDKQLF |
| | | IKRNQELLEKIEKQEAENHRLQQELQDARDQNELLEFRN |
| | | LELEERERRSPPFNLQIHPFSDGVSALQIYCMKEGVKDVN |
| | | IPDLIKQLDILGDNGNLRNEEQVAIIQASTVLSLAEKWIQQ |
| | | IEGAEAALHQKMMELESDMEQFCKIKGYLEEELDYRKQ |
| | | ALDQAYMRIQELEATLYNALQQETVIKFGELLSEKQQEE |
| | | LRTAVEKLRRQMLRKSREYDCQILQERMELLQQAHQRIR |
| | | DLEDKTDIQKRQIKDLEEKSNRKHG |
| SEQ ID NO: 149 | amino acid sequence of | MGEKSENCGVPEDLLNGLKVTDTQEAECAGPPVPDPKN |
| | Homo sapiens TTC1 | QHSQSKLLRDDEAHLQEDQGEEECFHDCSASFEEEPGAD |
| | | KVENKSNEDVNSSELDEEYLIELKKNMSDEEKQKRREES |
| | | TRLKEEGNEQFKKGDYIEAESSYSRALEMCPSCFQKERSI |
| | | LFSNRAAARMKQDKKEMAINDCSKAIQLNPSYIRAILRR |
| | | AELYEKTDKLDEALEDYKSILEKDPSIHQAREACMRLPK |
| | | QIEERNERLKEEMLGKLKDLGNLVLRPFGLSTENFQIKQD |
| | | SSTGSYSINFVQNPNNNR″ |

What is claimed is:

1. A method comprising:
a. contacting a sample from a subject suspected of having a cancer with one or more isolated and purified tumor-associated proteins, wherein the one or more isolated and purified tumor-associated proteins comprise at least one selected from the group consisting of: tripartate motif containing 33 (TRIMM33), BCL tumor suppressor 7A (BCL7A), Prostatic acid phosphatase (ACPP), mitochondrial transcription termination factor 4 (MTERF4), Sulfotransferase family cytosolic 2B member 1 (SULT2B1), Tetratricopeptide repeat protein 1 (TTC1), a polypeptide having at least 80 percent sequence identity with a sequence as shown in one of SEQ ID NO: 121, SEQ ID NO: 124, SEQ ID NO: 126, SEQ ID NO: 141, SEQ ID NO: 142, SEQ ID NO: 149, and any combination thereof.

2. The method of claim 1, further comprising detecting a binding of at least one of the one or more isolated and purified tumor-associated proteins to one or more moieties.

3. The method of claim 1 or 2, wherein the cancer is a lung cancer.

4. The method of claim 3, further comprising characterizing the subject as having an increased probability of having the lung cancer.

5. The method of claim 4, wherein the characterizing is performed with a sensitivity of at least about 60%.

6. The method of claim 4 or 5, wherein the characterizing is performed with a specificity of at least about 85%.

7. The method of any one of claims 1-6, wherein the one or more isolated and purified tumor-associated proteins comprise from 1 to about 100 sequentially unique proteins.

8. The method of any one of claims 1-6 wherein the one or more isolated and purified tumor-associated proteins comprise from 1 to about 10 sequentially unique proteins.

9. The method of any one of claims 1-8, wherein the one or more isolated and purified tumor-associated proteins comprise a tumor-associated protein that binds to immunoglobulin A (IgA) and a tumor-associated protein that binds to immunoglobulin G (IgG).

10. The method of any one of claims 1-9, wherein the one or more isolated and purified tumor-associated proteins comprise: TRIM33, BCL7A, DDX4, CTAG1A, MAGEC2, and MTERF4.

11. The method of any one of claims 1-9, wherein the one or more isolated and purified tumor-associated proteins comprise: BCL7A, DDX4, CTAG1A, MAGEC2, MTERF4, and NUDT14.

12. The method of any one of claims 1-9, wherein the one or more isolated and purified tumor-associated proteins comprise: TRIM33, BCL7A, CTAG1A, MAGEC2, MTERF4, and C1QTNF1.

13. The method of any one of claims 1-9, wherein the one or more isolated and purified tumor-associated proteins comprise: BCL7A, DDX4, CTAG1A, MAGEC2, DDX4, and RALGDS.

14. The method of any one of claims 1-9, wherein the one or more isolated and purified tumor-associated proteins comprise: TRIM33, BCL7A, DDX4, CTAG1A, MTERF4, and C1QTNF1.

15. The method of any one of claims 1-9, wherein the one or more isolated and purified tumor-associated proteins comprise: BCL7A, DDX4, CTAG1A, MAGEC2, MTERF4, and APH1A.

16. The method of any one of claims 1-9, wherein the one or more isolated and purified tumor-associated proteins comprise: BCL7A, DDX4, CTAG1A, MAGEC2, MTERF4, and N4BP1.

17. The method of any one of claims 1-9, wherein the one or more isolated and purified tumor-associated proteins comprise: BCL7A, DDX4, CTAG1A, MAGEC2, MTERF4, and SSBP4.

18. The method of any one of claims 1-9, wherein the one or more isolated and purified tumor-associated proteins comprise: TRIM33, BCL7A, DDX4, CTAG1A, APH1A, and C1QTNF1.

19. The method of any one of claims 1-9, wherein the one or more isolated and purified tumor-associated proteins comprise: TRIM33, BCL7A, DDX4, CTAG1A, KCNRG, and C1QTNF1.

20. The method of any one of claims 1-9, wherein the one or more isolated and purified tumor-associated proteins comprise: TRIM33, BCL7A, DDX4, CTAG1A, C1QTNF1, and SSBP4.

21. The method of any one of claims 1-9, wherein the one or more isolated and purified tumor-associated proteins comprise: BCL7A, DDX4, CTAG1A, MAGEC2, MTERF4, and YP021.

22. The method of any one of claims 1-9, wherein the one or more isolated and purified tumor-associated proteins comprise: BCL7A, DDX4, CTAG1A, MAGEC2, MTERF4, C1QTNF1.

23. The method of any one of claims 1-9, wherein the one or more isolated and purified tumor-associated proteins comprise: BCL7A, DDX4, CTAG1A, MAGEC2, MTERF4, and NUMBL.

24. The method of any one of claims 1-9, wherein the one or more isolated and purified tumor-associated proteins comprise: BCL7A, DDX4, CTAG1A, MAGEC2, MTERF4, and SULT2B1.

25. The method of any one of claims 1-9, wherein the one or more isolated and purified tumor-associated proteins comprise: BCL7A, DDX4, CTAG1A, MTERF4, ACPP, and C1QTNF1.

26. The method of any one of claims 1-9, wherein the one or more isolated and purified tumor-associated proteins comprise: TRIM33-IgA, SSBP4-IgA, TP53-IgG, NSG1-IgG and CTAG1A-IgG.

27. The method of any one of claims 1-9, wherein the one or more isolated and purified tumor-associated proteins comprise: TRIM33-IgA, MTERF4-IgA, TP53-IgG, MAGEC2-IgG, and CTAG1A-IgG.

28. The method of any one of claims 2-25, wherein the one or more moieties comprise an antibody.

29. The method of any one of claim 28, wherein the antibody is an autoantibody.

30. The method of any one of claims 2-29, wherein the detecting comprises detecting a signal.

31. The method of any one of claims 2-30, wherein the detecting comprises an immunoassay.

32. The method of any one of claims 2-31, wherein the signal has a Z score greater than a cutoff value of 1.

33. The method of any one of claims 2-32, wherein the method is a method of screening for the presence or absence of the cancer.

34. The method of any one of claims 2-32, wherein the detecting is indicative of the subject having the cancer.

35. The method of claim 34, further comprising validating the presence of the cancer.

36. The method of claim 34 or 35, further comprising monitoring the cancer in the subject.

37. The method of any one of claim 34, wherein the method distinguishes the cancer from a second disease.

38. The method of claim 37, wherein the detecting is indicative of the subject having the cancer, wherein the patient displays at least one symptom that is characteristic of both the cancer and the second disease.

39. The method of claim 37 and 38, wherein the subject is suspected of having the second disease.

40. The method of any one of claims 37-39, wherein the subject has the cancer and does not have the second disease.

41. The method of any one of claims 37-40, wherein the second disease is benign lung lesions (LBL), pneumonia, chronic obstructive pulmonary disease (COPD), pulmonary tuberculosis, or a second cancer.

42. The method of claim 41, wherein the second cancer is rectal cancer, liver cancer, cervical cancer, esophagus cancer, or gastric cancer.

43. The method of any one of claims 2-42, wherein the lung cancer is small-cell lung cancer (SCLC) or non-small cell lung cancer (NSCLC).

44. The method of any one of claims 1-43, wherein the subject has been previously diagnosed with the cancer.

45. The method of any one of claims 1-44, further comprising selecting a therapeutic based at least in part on a result of the method.

46. The method of any one of claims 1-44, further comprising treating the cancer in the subject.

47. The method of claim 46, wherein the treating comprises one or more of: surgery, chemotherapy, radiation therapy, immunotherapy, targeted therapy, hormone therapy, stem cell transplant, and precision medicine

48. The method of any one of claims 1-47, wherein the method is comprised in a companion diagnostic test.

49. The method of any one of claims 1-48, further comprising communicating the detecting via a communication medium.

50. The method of claim 49, wherein the communication medium comprises an electronic medium.

51. The method of any one of claims 1-50, wherein the subject is a human.

52. The method of any one of claims 1-51, wherein the sample comprises a body fluid.

53. The method of claim 52, wherein the body fluid comprises whole blood, plasma, serum, sputum, or pleural fluid.

54. The method of any one of claims 1-53, wherein the contacting is performed on a solid support.

55. The method of any one of claims 1-54, wherein the contacting or the detecting is performed using a portable device.

56. An in vitro composition that comprises:
a. at least two tumor-associated proteins, wherein the at least two isolated and purified tumor-associated proteins comprise at least one selected from the group consisting of: tripartate motif containing 33 (TRIMM33), BCL tumor suppressor 7A (BCL7A), Prostatic acid phosphatase (ACPP), mitochondrial transcription termination factor 4 (MTERF4), Sulfotransferase family cytosolic 2B member 1 (SULT2B1), Tetratricopeptide repeat protein 1 (TTC1), a polypeptide having at least 80 percent sequence identity with a sequence as shown in one of SEQ ID NO: 121, SEQ ID NO: 124, SEQ ID NO: 126, SEQ ID NO: 141, SEQ ID NO: 142, SEQ ID NO: 149, and any combination thereof, and
b. a moiety bound to at least one of the at least two tumor-associated proteins, wherein the moiety is present in a sample obtained from a subject suspected of having a cancer.

57. The in vitro composition of claim 56, wherein the at least two tumor-associated proteins are isolated and purified.

58. The in vitro composition of claim 56 or 57, wherein the moiety is an antibody.

59. The in vitro composition of claim 58, wherein the antibody is an autoantibody.

60. The in vitro composition of any one of claims 56-59, wherein the sample comprises a body fluid.

61. The in vitro composition of claim 60, wherein the body fluid comprises whole blood, plasma, serum, sputum, or pleural fluid.

62. The in vitro composition of any one of claims 56-61, wherein the subject is a human.

63. The in vitro composition of any one of claims 56-62, wherein the cancer is a lung cancer.

64. A solid support that comprises: two or more isolated and purified tumor-associated proteins attached thereto, wherein at least two of the two or more isolated and purified tumor-associated proteins are selected from the group consisting of: tripartate motif containing 33 (TRIMM33), BCL tumor suppressor 7A (BCL7A), Prostatic acid phosphatase (ACPP), mitochondrial transcription termination factor 4 (MTERF4), Sulfotransferase family cytosolic 2B member 1 (SULT2B1), Tetratricopeptide repeat protein 1 (TTC1), a polypeptide having at least 80 percent sequence identity with a sequence as shown in one of SEQ ID NO: 121, SEQ ID NO: 124, SEQ ID NO: 126, SEQ ID NO: 141, SEQ ID NO: 142, SEQ ID NO: 149, and any combination thereof.

65. The solid support of claim 64, wherein the two or more isolated and purified tumor-associated proteins comprise from 1 to about 100 sequentially unique proteins.

66. The solid support of claim 64, wherein the two or more isolated and purified tumor-associated proteins comprise from 1 to about 10 sequentially unique proteins.

67. A device that comprises:
a. a memory that stores executable instructions; and
b. a processor that executes the executable instructions such that the device performs the method of any one of claims 1-66.

68. A device that comprises:
a. the solid support of any one of claims 64-66, and
b. a processor for detecting a signal, wherein the signal is indicative of a binding of an autoantibody to at least one of the two or more isolated and purified tumor-associated proteins.

69. The device of claim 68, wherein the signal is at least about two standard deviations greater than a control signal.

70. A kit that comprises the solid support of any one of claims 64-66 and instructions for use thereof.

71. A kit that comprises the device of any one of claims 67-69 and instructions for use thereof.
